(19) United States
(12) Patent Application Publication
(10) Pub. No.: US 2009/0118261 A1
(43) Pub. Date: May 7, 2009

(54) QUINAZOLINONE DERIVATIVES AND THEIR USE AS B-RAF INHIBITORS

(75) Inventors: Brian Aquila, Waltham, MA (US); Leslie Dakin, Waltham, MA (US); Jayachandran Ezhuthachan, Waltham, MA (US); Stephen Lee, Waltham, MA (US); Paul Lyne, Waltham, MA (US); Timothy Pontz, Waltham, MA (US); XiaoLan Zheng, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/574,031

(22) PCT Filed: Aug. 26, 2005
(86) PCT No.: PCT/GB05/03334

§ 371 Date: Feb. 21, 2007

Related U.S. Application Data
(60) Provisional application No. 60/605,762, filed on Aug. 31, 2004.

(51) Int. Cl. A61K 31/55; C07D 239/72; A61K 31/517; C07D 413/02; A61K 31/5377; A61P 35/00; C07D 471/02; A61K 31/519; A61K 31/496; C07D 403/02

(52) U.S. Cl. 514/217.06; 544/287; 514/266.3; 544/116; 514/234.5; 544/279; 514/264.1; 514/252.17; 540/600

(57) ABSTRACT

The invention relates to chemical compounds of the formula (I): or pharmaceutically acceptable salts thereof, which possess B Raf inhibitory activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

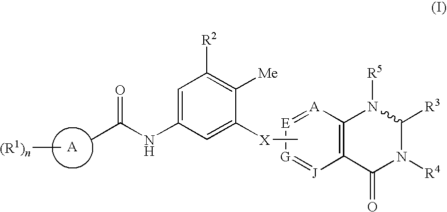

The invention relates to chemical compounds, or pharmaceutically acceptable salts thereof, which possess B-Raf inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

The classical Ras, Raf, MAP protein kinase/extracellular signal-regulated kinase kinase (MEK), extracellular signal-regulated kinase (ERK) pathway plays a central role in the regulation of a variety of cellular functions dependent upon cellular context, including cellular proliferation, differentiation, survival, immortalization and angiogenesis (reviewed in Peyssonnaux and Eychene, Biology of the Cell, 2001, 93, 3-62). In this pathway, Raf family members are recruited to the plasma membrane upon binding to guanosine triphosphate (GTP) loaded Ras resulting in the phosphorylation and activation of Raf proteins. Activated. Rafs then phosphorylate and activate MEKs, which in turn phosphorylate and activate ERKs. Upon activation, ERKs translocate from the cytoplasm to the nucleus resulting in the phosphorylation and regulation of activity of transcription factors such as Elk-1 and Myc.

The Ras/Raf/MEK/ERK pathway has been reported to contribute to the tumorigenic phenotype by inducing immortalisation, growth factor-independent growth, insensitivity to growth-inhibitory signals, ability to invade and metastasis, stimulating angiogenesis and inhibition of apoptosis (reviewed in Kolch et al., Exp. Rev. Mol. Med., 2002, 25 Apr., http://www.expertreviews.org/02004386h.htm). In fact, ERK phosphorylation is enhanced in approximately 30% of all human tumours (Hoshino et al., Oncogene, 1999, 18, 813-822). This may be a result of overexpression and/or mutation of key members of the pathway.

Three Raf serine/threonine protein kinase isoforms have been reported Raf-1/c-Raf, B-Raf and A-Raf (reviewed in Mercer and Pritchard, Biochim. Biophys. Acta, 2003, 1653, 25-40), the genes for which are thought to have arisen from gene duplication. All three Raf genes are expressed in most tissues with high-level expression of B-Raf in neuronal tissue and A-Raf in urogenital tissue. The highly homologous Raf family members have overlapping but distinct biochemical activities and biological functions (Hagemann and Rapp, Expt. Cell Res. 1999, 253, 34-46). Expression of all three Raf genes is required for normal murine development however both c-Raf and B-Raf are required to complete gestation. B-Raf −/− mice die at E12.5 due to vascular haemorrhaging caused by increased apoptosis of endothelial cells (Wojnowski et al., Nature Genet., 1997, 16, 293-297). B-Raf is reportedly the major isoform involved in cell proliferation and the primary target of oncogenic Ras. Activating somatic missense mutations have been identified exclusively for B-Raf, occurring with a frequency of 66% in malignant cutaneous melanomas (Davies et al., Nature, 2002, 417, 949-954) and also present in a wide range of human cancers, including but not limited to papillary thyroid tumours (Cohen et al., J. Natl. Cancer Inst., 2003, 95, 625-627), cholangiocarcinomas (Tannapfel et al., Gut, 2003, 52, 706-712), colon and ovarian cancers (Davies et al., Nature, 2002, 417, 949-954). The most frequent mutation in B-Raf (80%) is a glutamic acid for valine substitution at position 600. These mutations increase the basal kinase activity of B-Raf and are thought to uncouple Raf/MEK/ERK signalling from upstream proliferation drives including Ras and growth factor receptor activation resulting in constitutive activation of ERK. Mutated B-Raf proteins are transforming in NIH3T3 cells (Davies et al., Nature, 2002, 417, 949-954) and melanocytes (Wellbrock et al., Cancer Res., 2004, 64, 2338-2342) and have also been shown to be essential for melanoma cell viability and transformation (Hingorani et al., Cancer Res., 2003, 63, 5198-5202). As a key driver of the Raf/MEK/ERK signalling cascade, B-Raf represents a likely point of intervention in tumours dependent on this pathway.

Accordingly, the present invention provides a compound of formula (I):

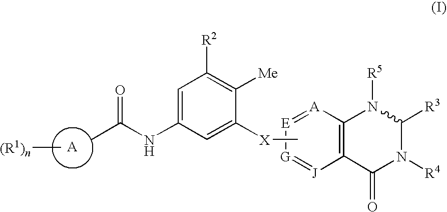

wherein:

Ring A is carbocyclyl or heterocyclyl; wherein if said heterocyclyl contains an —NH— moiety that nitrogen may be optionally substituted by a group selected from R⁶;

R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, N—(C₁₋₆alkoxy)sulphamoyl, N—(C₁₋₆alkyl)-N—(C₁₋₆alkoxy)sulphamoyl, C₁₋₆alkylsulphonylamino, carbocyclyl-R⁷— or heterocyclyl-R⁸—; wherein R¹ may be optionally substituted on carbon by one or more R⁹; and wherein if said heterocyclyl contains an —NH— moiety that nitrogen may be optionally substituted by a group selected from R¹⁰;

n is selected from 0-4; wherein the values of R¹ may be the same or different;

R² is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹¹— or heterocyclyl-R¹²—; wherein R² may be optionally substituted on carbon by one or more R¹³; and wherein if said heterocyclyl contains an —NH— moiety that nitrogen may be optionally substituted by a group selected from R¹⁴;

X is NR¹⁵ or O;

one of A, E, G and J is C which is attached to X of formula (I); the other three are independently selected from CR¹⁶ or N;

R³ and R¹⁶ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R³ and R¹⁶ independently of each other may be optionally substituted on carbon by one or more R¹⁹; and wherein if said heterocyclyl contains an —NH— moiety that nitrogen may be optionally substituted by a group selected from R²⁰;

R⁴, R⁵ and R¹⁵ are independently selected from hydrogen, C₁₋₆alkyl, C₁₋₆alkanoyl, C₁₋₆alkylsulphonyl, C₁₋₆alkoxycarbonyl, carbamoyl, carbocyclyl, heterocyclyl, N—(C₁₋₆alkyl)carbamoyl and N,N—(C₁₋₆alkyl)carbamoyl; wherein R⁴, R⁵ and R¹⁵ independently of each other may be optionally substituted on carbon by one or more R²¹;

the bond [symbol] between the —NR⁵— and —CR³— of formula (I) is either (i) a single bond wherein R⁵ is as defined above, or (ii) a double bond wherein R⁵ is absent;

R⁹, R¹³, R¹⁹ and R²¹ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R²²— or heterocyclyl-R²³—; wherein R⁹, R¹³, R¹⁹ and R²¹ independently of each other may be optionally substituted on carbon by one or more R²⁴; and wherein if said heterocyclyl contains an —NH— moiety that nitrogen may be optionally substituted by a group selected from R²⁵;

R⁷, R⁸, R¹¹, R¹², R¹⁷, R¹⁸, R²² and R²³ are independently selected from a direct bond, —O—, —N(R²⁶)—, —C(O)—, —N(R²⁷)C(O)—, —C(O)N(R²⁸)—, —S(O)ₛ—, —SO₂N(R²⁹)— or —N(R³⁰)SO₂—; wherein R²⁶, R²⁷, R²⁸, R²⁹ and R³⁰ is hydrogen, C₁₋₆alkoxycarbonyl or C₁₋₆alkyl and s is 0-2;

R⁶, R¹⁰, R¹⁴, R²⁰ and R²⁵ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R²⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In a further aspect of the present invention there is provided a compound of formula (I) (as depicted above) wherein:

Ring A is carbocyclyl or heterocyclyl; wherein if said heterocyclyl contains an —NH— moiety that nitrogen may be optionally substituted by a group selected from R⁶;

R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁷— or heterocyclyl-R⁸—; wherein R¹ may be optionally substituted on carbon by one or more R⁹; and wherein if said heterocyclyl contains an —NH— moiety that nitrogen may be optionally substituted by a group selected from R¹⁰;

n is selected from 0-4; wherein the values of R¹ may be the same or different;

R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹¹— or heterocyclyl-R¹²—; wherein R² may be optionally substituted on carbon by one or more R¹³; and wherein if said heterocyclyl contains an —NH— moiety that nitrogen may be optionally substituted by a group selected from R¹⁴;

X is NR¹⁵ or O;

one of A, E, G and J is C which is attached to X of formula (I); the other three are independently selected from CR¹⁶ or N;

R³ and R¹⁶ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R³ and R¹⁶ independently of each other may be optionally substituted on carbon by one or more R¹⁹; and wherein if said heterocyclyl contains an —NH— moiety that nitrogen may be optionally substituted by a group selected from R²⁰;

R⁴, R⁵ and R¹⁵ are independently selected from hydrogen, C₁₋₆alkyl, C₁₋₆alkanoyl, C₁₋₆alkylsulphonyl, C₁₋₆alkoxycarbonyl, carbamoyl, N—(C₁₋₆alkyl)carbamoyl and N,N—(C₁₋₆alkyl)carbamoyl; wherein R⁴, R⁵ and R¹⁵ independently of each other may be optionally substituted on carbon by one or more R²¹;

the bond [symbol] between the —NR⁵— and —CR³— of formula (I) is either (i) a single bond wherein R⁵ is as defined above, or (ii) a double bond wherein R⁵ is absent;

R⁹, R¹³, R¹⁹ and R²¹ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²²— or heterocyclyl-R²³—; wherein R⁹, R¹³, R¹⁹ and R²¹ independently of each other may be optionally substituted on carbon by one or more R²⁴; and wherein if said heterocyclyl contains an —NH— moiety that nitrogen may be optionally substituted by a group selected from R²⁵;

R⁷, R⁸, R¹¹, R¹², R¹⁷, R¹⁸, R²² and R²³ are independently selected from a direct bond, —O—, —N(R²⁶)—, —C(O)—, —N(R²⁷)C(O)—, —C(O)N(R²⁸)—, —S(O)ₛ—, —SO₂N(R²⁹)— or —N(R³⁰)SO₂—; wherein R²⁶, R²⁷, R²⁸, R²⁹ and R³⁰ is hydrogen or C₁₋₆alkyl and s is 0-2;

R⁶, R¹⁰, R¹⁴, R²⁰ and R²⁵ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R²⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In this specification the term “alkyl” includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, and a ring sulphur atom may be optionally oxidised to form the S-oxides. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. A particular example of the term “heterocyclyl” is pyrazolyl. In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂carbamoyl” are N,N—(C₁₋₄alkyl)₂carbamoyl, dimethylaminocarbonyl and methylethylaminocarbonyl. Examples of “C₁₋₆alkylsulphonyl” are mesyl, ethylsulphonyl and isopropylsulphonyl. Examples of “C₁₋₆alkylsulphonylamino” are mesylamino, ethylsulphonylamino and isopropylsulphonylamino. Examples of “N—(C₁₋₆alkoxy)sulphamoyl” include N-(methoxy)sulphamoyl and N-(ethoxy)sulphamoyl. Examples of “N—(C₁₋₆alkyl)-N—(C₁₋₆alkoxy)sulphamoyl” N-(methyl)-N-(methoxy)sulphamoyl and N-(propyl)-N-(ethoxy)sulphamoyl.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess B-Raf inhibitory activity. The invention further relates to any and all tautomeric forms of the compounds of the formula (I) that possess B-Raf inhibitory activity.

It is also to be understood that certain compounds of the formula (I) can exist in solvated as well as unsolvated forms such as, for example, hydrated forms. It is to be understood that the invention encompasses all such solvated forms which possess B-Raf inhibitory activity.

Particular values of variable groups are as follows. Such values may be used where appropriate with any of the definitions, claims or embodiments defined hereinbefore or hereinafter.

Ring A is carbocyclyl.

Ring A is heterocyclyl.

Ring A heterocyclyl; wherein if said heterocyclyl contains an —NH— moiety that nitrogen may be optionally substituted by a group selected from R⁶.

Ring A heterocyclyl; wherein if said heterocyclyl contains an —NH— moiety that nitrogen may be optionally substituted by a group selected from R⁶; wherein R⁶ is C₁₋₆alkyl.

Ring A is phenyl, thienyl, pyridyl or thiazolyl.

Ring A is phenyl, thienyl, pyridyl, thiazolyl, isoxazolyl, furyl, 1,3-benzodioxolyl, pyrazolyl, indolyl, 2,3-dihydrobenzofuranyl, imidazo[1,2-a]pyridinyl or pyrimidinyl; wherein said pyrazolyl may be optionally substituted on nitrogen by a group selected from R⁶; wherein R⁶ is C₁₋₆alkyl.

Ring A is phenyl, thienyl, pyridyl, thiazolyl, isoxazolyl, furyl, 1,3-benzodioxolyl, pyrazolyl, indolyl, 2,3-dihydrobenzofuranyl, imidazo[1,2-a]pyridinyl or pyrimidinyl; wherein said pyrazolyl may be optionally substituted on nitrogen by a group selected from R⁶; wherein R⁶ is methyl or t-butyl.

Ring A is phenyl, thien-2-yl, thien-3-yl, pyrid-2-yl, pyrid-3-yl, pyrid-4-yl, thiazol-4-yl, isoxazol-3-yl, 1,3-benzodioxol-5-yl, fur-2-yl, 1-methylpyrazol-3-yl, 1-methylpyrazol-5-yl, 1-t-butylpyrazol-5-yl, indol-5-yl, indol-6-yl, 2,3-dihydrobenzofuran-7-yl, imidazo[1,2-a]pyridin-2-yl or pyrimidin-4-yl.

Ring A is phenyl.

R¹ is a substituent on carbon and is selected from halo, hydroxy, C₁₋₆alkyl, C₁₋₆alkoxy or C₁₋₆alkoxycarbonyl; wherein R¹ may be optionally substituted on carbon by one or more R⁹; wherein

R⁹ is selected from halo, cyano, N,N—(C₁₋₆alkyl)₂amino or heterocyclyl-R²³—; and

R²³ is selected from a direct bond.

R¹ is a substituent on carbon and is selected from halo, hydroxy, cyano, sulphamoyl, C₁₋₆alkyl, C₂₋₆alkenyl, C₂₋₆alkynyl, C₁₋₆alkoxy, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, N—(C₁₋₆alkyl)-N—(C₁₋₆alkoxy)sulphamoyl, carbocyclyl-R⁷— or heterocyclyl-R⁸—; wherein R¹ may be optionally substituted on carbon by one or more R⁹; and wherein if said heterocyclyl contains an —NH— moiety that nitrogen may be optionally substituted by a group selected from R¹⁰;

R⁹ is selected from halo, cyano, hydroxy, carboxy, C₁₋₆alkyl, C₁₋₆alkoxy, N,N—(C₁₋₆alkyl)₂amino, N—(C₁₋₆alkyl)carbamoyl, N,N—(C₁₋₆alkyl)₂carbamoyl, C₁₋₆alkylS(O)ₐ wherein a is 0 to 2, carbocyclyl-R²²— or heterocyclyl-R²³—; wherein R⁹ may be optionally substituted on carbon by one or more R²⁴; and wherein if said heterocyclyl contains an —NH— moiety that nitrogen may be optionally substituted by a group selected from R²⁵;

R⁷, R⁸, R²² and R²³ are independently selected from a direct bond, —O—, —N(R²⁶)—, —C(O)—, —S(O)ₛ— or —N(R³⁰)SO₂—; wherein R²⁶ and R³⁰ are independently selected from hydrogen or C₁₋₆alkoxycarbonyl; and s is 2;

R¹⁰ and R²⁵ are independently-selected from C₁₋₆alkyl;

R²⁴ is hydroxy.

R¹ is a substituent on carbon and is selected from chloro, hydroxy, methyl, isopropyl, methoxy, ethoxy or methoxycarbonyl; wherein R¹ may be optionally substituted on carbon by one or more R⁹; wherein

R⁹ is selected from fluoro, cyano, dimethylamino or pyrrolidinyl.

R¹ is a substituent on carbon and is selected from fluoro, chloro, bromo, hydroxy, cyano, sulphamoyl, methyl, ethyl, propyl, isopropyl, 1,1-dimethylpropyl, t-butyl, ethenyl, 1,1-dimethylprop-2-ynyl, 3,3-dimethylbut-1-ynyl, propynyl, 3-methylbut-1-ynyl, methoxy, ethoxy, propoxy, N,N-dimethylcarbamoyl, mesyl, methoxycarbonyl, N-(methyl)sulphamoyl, N-propyl-N-methylsulphamoyl, N,N-dimethylsulphamoyl, N-(methyl)-N-(methoxy)sulphamoyl, cyclopropyl-R⁷—, azetidinyl-R⁸—, morpholino-R⁸— or piperidinyl-R⁸—; wherein R¹ may be optionally substituted on carbon by one or more R⁹; and wherein said piperidinyl may be optionally substituted on nitrogen by a group selected from R¹⁰;

R⁹ is selected from fluoro, cyano, hydroxy, carboxy, methyl, methoxy, dimethylamino, N-(methyl)carbamoyl, N,N-dimethylcarbamoyl, methylthio, mesyl, cyclopropyl-R²²—, piperazinyl-R²³—, morpholino-R²³—, tetrahydrofuranyl-R²³—, piperidinyl-R²³—, azepanyl-R²³— or pyrrolidinyl-R²³—; wherein R⁹ may be optionally substituted on carbon by one or more R²⁴; and wherein said piperazinyl or pyrrolidinyl may be optionally substituted on nitrogen by a group selected from R²⁵;

R⁷, R⁸, R²² and R²³ are independently selected from a direct bond, —O—, —N(R²⁶)—, —C(O)—, —S(O)ₛ— or —N(R³)SO₂—; wherein R²⁶ and R³⁰ are independently selected from hydrogen or t-butoxycarbonyl; and s is 2;

R¹⁰ and R²⁵ are selected from methyl;

R²⁴ is hydroxy.

R¹ is a substituent on carbon and is selected from 1-methyl-1-cyanoethyl, trifluoromethyl, chloro, methoxycarbonyl, 2-dimethylaminoethoxy, methoxy, hydroxy and 2-pyrrolidin-1-ylethoxy.

R¹ is a substituent on carbon and is selected from fluoro, chloro, bromo, hydroxy, cyano, sulphamoyl, methyl, trifluoromethyl, cyclopropylaminomethyl, methylthiomethyl, mesylmethyl, dimethylaminomethyl, 1-(cyclopropyl)-1-hydroxymethyl, N-cyclopropyl-N-(t-butoxycarbonyl)aminomethyl, 1-methylpiperazin-4-ylmethyl, 1-hydroxy-1-cyclopropylethyl, 1-methyl-1-cyanoethyl, 2-methoxy-1,1-dimethylethyl, 1-carboxy-1-methylethyl, 1,1-difluoroethyl, 2-(dimethylamino)-1,1-dimethyl-2-oxoethyl, 3-(dimethylamino)propyl, 1,1-dimethylpropyl, t-butyl, methoxy, N-methylcarbamoylmethoxy, 2-(dimethylamino)ethoxy, 2-(pyrrolidin-1-yl)ethoxy, 2-(methoxy)ethoxy, 2-(1-methylpyrrolidin-2-yl)ethoxy, 2-(piperidin-1-yl)ethoxy, 2-(azepan-1-yl)ethoxy, 2-(morpholino)ethoxy, 3-(1-methylpiperazin-4-yl)propoxy, methoxycarbonyl, morpholinocarbonyl, N,N-dimethylsulphamoyl, N-(2,3-dihydroxypropyl)-N-methylsulphamoyl, N-(methyl)-N-(methoxy)sulphamoyl, 1-methylpiperidin-4-yloxy, N,N-dimethylcarbamoyl, cyclopropyl, piperidin-1-yl, morpholino, 1-cyclopropylethenyl, 3-(4-methylpiperazin-1-yl)prop-1-yn-1-yl, 3,3-dimethylbut-1-yn-1-yl, cyclopropylethynyl, 3-hydroxy-3-methylbut-1-yn-1-yl, 1,1-dimethylprop-2-yn-1-yl, 3-(dimethylamino)prop-1-yn-1-yl, mesyl, cyclopropylaminosulphonyl, azetidin-1-ylsulphonyl, morpholinosulphonyl, tetrahydrofur-2-ylmethylaminosulphonyl, 2-(hydroxymethyl)piperidin-1-ylsulphonyl, 3-(hydroxymethyl)piperidin-1-ylsulphonyl or 4-(hydroxymethyl)piperidin-1-ylsulphonyl.

R¹ is a substituent on carbon and is selected from 1-methyl-1-cyanoethyl.

n is selected from 0-2; wherein the values of R¹ may be the same or different.

n is selected from 1-2; wherein the values of R¹ may be the same or different.

n is 2.

n is 1.

n is 0.

R² is selected from hydrogen.

X is NR¹⁵.

X is O.

X is NR¹⁵ or O; wherein

R¹⁵ is selected from hydrogen or C₁₋₆alkyl; wherein R¹⁵ may be optionally substituted on carbon by one or more R²¹;

R²¹ is selected from carbocyclyl-R²²—;

R²² is a direct bond.

X is NR¹⁵ or O; wherein

R¹⁵ is selected from hydrogen or methyl; wherein R¹⁵ may be optionally substituted on carbon by one or more R²¹;

R²¹ is selected from cyclopropyl.

X is NR¹⁵ or O; wherein

R¹⁵ is selected from hydrogen, methyl or cyclopropylmethyl.

one of A, E, G and J is C which is attached to X of formula (I); the other three are all CR¹⁶ or two are CR¹⁶ and one is N.

one of A, E, G and J is C which is attached to X of formula (I); the other three are all CR¹⁶ or two are CR¹⁶ and one is N; wherein R¹⁶ is hydrogen.

G is C which is attached to X of formula (I).

E is C which is attached to X of formula (I).

A and J are CR¹⁶ wherein R¹⁶ is hydrogen.

R¹⁶ is hydrogen.

E is CR¹⁶.

E is N.

G is CR¹⁶.

R³ is hydrogen or C₁₋₆alkyl.

R³ is selected from hydrogen, C₁₋₆alkyl, N—(C₁₋₆alkyl)amino, N,N—(C₁₋₆alkyl)₂amino or C₁₋₆alkylS(O)ₐ wherein a is 0; wherein R³ may be optionally substituted on carbon by one or more R¹⁹; wherein

R¹⁹ is hydroxy.

R³ is selected from hydrogen, methyl, N-(ethyl)amino, N,N-dimethylamino or methylthio; wherein R³ may be optionally substituted on carbon by one or more R¹⁹; wherein

R¹⁹ is hydroxy.

R³ is hydrogen or methyl

R³ is selected from hydrogen, methyl, N-(2-hydroxyethyl)amino, N,N-dimethylamino or methylthio.

R⁴ is selected from hydrogen or C₁₋₆alkyl; wherein R⁴ may be optionally substituted on carbon by one or more R²¹; wherein

R²¹ is selected from hydroxy, carbocyclyl-R²²— or heterocyclyl-R²³—; wherein R²¹ may be optionally substituted on carbon by one or more R²⁴;

R²² and R²³ are a direct bond;

R²⁴ is methyl.

R⁴ is selected from hydrogen, C₁₋₆alkyl or carbocyclyl; wherein R⁴ may be optionally substituted on carbon by one or more R²¹;

R²¹ is selected from hydroxy, amino, C₁₋₆alkoxycarbonylamino, carbocyclyl-R²²— or heterocyclyl-R²³—; wherein R²¹ may be optionally substituted on carbon by one or more R²⁴; and wherein if said heterocyclyl contains an —NH— moiety that nitrogen may be optionally substituted by a group selected from R²⁵;

R²² and R²³ are a direct bond;

R²⁴ is methyl; and

R²⁵ is C₁₋₆alkyl or benzyloxycarbonyl.

R⁴ is selected from hydrogen, methyl, ethyl or propyl; wherein R⁴ may be optionally substituted on carbon by one or more R²¹; wherein

R²¹ is selected from hydroxy, cyclopropyl, 1,3-dioxolanyl or morpholino; wherein R²¹ may be optionally substituted on carbon by one or more R²⁴;

R²⁴ is methyl.

R⁴ is selected from hydrogen, methyl, ethyl, propyl or cyclopropyl; wherein R⁴ may be optionally substituted on carbon by one or more R²¹;

R²¹ is selected from hydroxy, amino, t-butoxycarbonylamino, cyclopropyl, 1,3-dioxolan-4-yl, piperidinyl or morpholino; wherein R²¹ may be optionally substituted on carbon by one or more R²⁴; and wherein said piperidinyl may be optionally substituted on nitrogen by a group selected from R²⁵;

R²⁴ is methyl; and

R²⁵ is methyl or benzyloxycarbonyl.

R⁴ is hydrogen, methyl, ethyl, 3-morpholinopropyl, cyclopropylmethyl, 2,2-dimethyl-1,3-dioxolan-4-ylmethyl, 2,3-dihydroxypropyl or 2-hydroxyethyl.

R⁴ is selected from hydrogen, methyl, 1-methylpiperidin-3-ylmethyl, cyclopropylmethyl, 2,2-dimethyl-1,3-dioxolan-4-ylmethyl, piperidin-4-ylmethyl, 1-benzyloxycarbonylpipidin-4-ylmethyl, ethyl, 2-hydroxyethyl, 3-aminopropyl, 3-(t-butoxycarbonylamino)propyl, 3-morpholinopropyl, 2,3-dihydroxypropyl and cyclopropyl.

the bond [symbol] between the —NR⁵— and —CR³— of formula (I) is a single bond wherein R⁵ is as defined above.

the bond [symbol] between the —NR⁵— and —CR³— of formula (I) is a double bond wherein R⁵ is absent.

Therefore in a further aspect of the invention there is provided a compound of formula (I) (as depicted above) wherein:

Ring A is carbocyclyl or heterocyclyl;

R¹ is a substituent on carbon and is selected from halo, hydroxy, C₁₋₆alkyl, C₁₋₆alkoxy or C₁₋₆alkoxycarbonyl; wherein R¹ may be optionally substituted on carbon by one or more R⁹;

n is selected from 1-2; wherein the values of R¹ may be the same or different;

R² is selected from hydrogen;

R³ is hydrogen or C₁₋₆alkyl;

R⁴ is selected from hydrogen or C₁₋₆alkyl; wherein R⁴ may be optionally substituted on carbon by one or more R²¹;

X is NR¹⁵ or O;

one of A, E, G and J is C which is attached to X of formula (I); the other three are all CR¹⁶ or two are CR¹⁶ and one is N;

the bond [symbol] between the —NR⁵— and —CR³— of formula (I) is a double bond wherein R⁵ is absent;

R⁹ is selected from halo, cyano, N,N—(C₁₋₆alkyl)₂amino or heterocyclyl-R²³—;

R¹⁵ is selected from hydrogen or C₁₋₆alkyl; wherein R¹⁵ may be optionally substituted on carbon by one or more R²¹;

R²¹ is selected from hydroxy, carbocyclyl-R²²— or heterocyclyl-R²³—; wherein R²¹ may be optionally substituted on carbon by one or more R²⁴;

R²² and R²³ are a direct bond;

R²⁴ is methyl;

or a pharmaceutically acceptable salt thereof.

Therefore in a further aspect of the invention there is provided a compound of formula (I) (as depicted above) wherein:

Ring A is carbocyclyl or heterocyclyl;

R¹ is a substituent on carbon and is selected from halo, hydroxy, C₁₋₆alkyl, C₁₋₆alkoxy or C₁₋₆alkoxycarbonyl; wherein R¹ may be optionally substituted on carbon by one or more R⁹;

n is selected from 1-2; wherein the values of R¹ may be the same or different;

R² is selected from hydrogen;

R³ is hydrogen or C₁₋₆alkyl;

R⁴ is selected from hydrogen or C₁₋₆alkyl; wherein R⁴ may be optionally substituted on carbon by one or more R²¹;

X is NR¹⁵ or O;

one of A, E, G and J is C which is attached to X of formula (I); the other three are all CR¹⁶ or two are CR¹⁶ and one is N;

the bond [symbol] between the —NR⁵— and —CR³— of formula (I) is a double bond wherein R⁵ is absent;

R⁹ is selected from halo, cyano, N,N—(C₁₋₆alkyl)₂amino or heterocyclyl-R²³—;

R¹⁵ is selected from hydrogen or C₁₋₆alkyl; wherein R¹⁵ may be optionally substituted on carbon by one or more R²¹;

R¹⁶ is hydrogen;

R²¹ is selected from hydroxy, carbocyclyl-R²²— or heterocyclyl-R²³—; wherein R²¹ may be optionally substituted on carbon by one or more R²⁴;

R²² and R²³ are a direct bond;

R²⁴ is methyl;

or a pharmaceutically acceptable salt thereof.

Therefore in a further aspect of the invention there is provided a compound of formula (I) (as depicted above) wherein:

Ring A carbocyclyl or heterocyclyl; wherein if said heterocyclyl contains an —NH— moiety that nitrogen may be optionally substituted by a group selected from R⁶;

R¹ is a substituent on carbon and is selected from halo, hydroxy, cyano, sulphamoyl, C₁₋₆alkyl, C₂₋₆alkenyl, C₂₋₆alkynyl, C₁₋₆alkoxy, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, N—(C₁₋₆alkyl)-N—(C₁₋₆alkoxy)sulphamoyl, carbocyclyl-R⁷— or heterocyclyl-R⁸—; wherein R¹ may be optionally substituted on carbon by one or more R⁹; and wherein if said heterocyclyl contains an —NH— moiety that nitrogen may be optionally substituted by a group selected from R¹⁰;

n is selected from 0-2; wherein the values of R¹ may be the same or different;

R² is hydrogen;

X is NR¹⁵ or O;

one of A, E, G and J is C which is attached to X of formula (I); the other three are all CR¹⁶ or two are CR¹⁶ and one is N;

R³ is selected from hydrogen, C₁₋₆alkyl, N—(C₁₋₆alkyl)amino, N,N—(C₁₋₆alkyl)₂amino or C₁₋₆alkylS(O)ₐ wherein a is 0; wherein R³ may be optionally substituted on carbon by one or more R¹⁹;

R⁴ is selected from hydrogen, C₁₋₆alkyl or carbocyclyl; wherein R⁴ may be optionally substituted on carbon by one or more R²¹;

the bond [symbol] between the —NR⁵— and —CR³— of formula (I) is a double bond wherein R⁵ is absent;

R⁶ is C₁₋₆alkyl;

R⁹ is selected from halo, cyano, hydroxy, carboxy, C₁₋₆alkyl, C₁₋₆alkoxy, —N,N—(C₁₋₆alkyl)₂amino, N—(C₁₋₆alkyl)carbamoyl, N,N—(C₁₋₆alkyl)₂carbamoyl, C₁₋₆alkylS(O)ₐ wherein a is 0 to 2, carbocyclyl-R²²— or heterocyclyl-R²³—; wherein R⁹ may be optionally substituted on carbon by one or more R²⁴; and wherein if said heterocyclyl contains an —NH— moiety that nitrogen may be optionally substituted by a group selected from R²⁵;

R⁷, R⁸, R²² and R²³ are independently selected from a direct bond, —O—, —N(R²⁶)—, —C(O)—, —S(O)ₛ— or —N(R³⁰)SO₂—; wherein R²⁶ and R³⁰ are independently selected from hydrogen or C₁₋₆alkoxycarbonyl; and s is 2;

R¹⁰ and R²⁵ are independently selected from C₁₋₆alkyl or benzyloxycarbonyl;

R¹⁵ is selected from hydrogen or C₁₋₆alkyl; wherein R¹⁵ may be optionally substituted on carbon by one or more R²¹;

R¹⁶ is hydrogen;

R¹⁹ is hydroxy;

R²¹ is selected from hydroxy, amino, C₁₋₆alkoxycarbonylamino, carbocyclyl-R²²— or heterocyclyl-R²³—; wherein R²¹ may be optionally substituted on carbon by one or more R²⁴; and wherein if said heterocyclyl contains an —NH— moiety that nitrogen may be optionally substituted by a group selected from R²⁵; and

R²⁴ is hydroxy or methyl;

or a pharmaceutically acceptable salt thereof.

Therefore in a further aspect of the invention there is provided a compound of formula (I) (as depicted above) wherein:

Ring A is phenyl, thienyl, pyridyl or thiazolyl;

R¹ is a substituent on carbon and is selected from 1-methyl-1-cyanoethyl, trifluoromethyl, chloro, methoxycarbonyl, 2-dimethylaminoethoxy, methoxy, hydroxy and 2-pyrrolidin-1-ylethoxy;

n is selected from 1-2; wherein the values of R¹ may be the same or different;

R² is hydrogen;

X is NR¹⁵ or O;

one of A, E, G and J is C which is attached to X of formula (I); the other three are all CR¹⁶ or two are CR¹⁶ and one is N;

R³ is hydrogen or methyl;

R⁴ is hydrogen, methyl, ethyl, 3-morpholinopropyl, cyclopropylmethyl, 2,2-dimethyl-1,3-dioxolan-4-ylmethyl, 2,3-dihydroxypropyl or 2-hydroxyethyl; and

the bond [symbol] between the —NR⁵— and —CR³— of formula (I) is a double bond wherein R⁵ is absent;

R¹⁵ is selected from hydrogen, methyl or cyclopropylmethyl;

or a pharmaceutically acceptable salt thereof.

Therefore in a further aspect of the invention there is provided a compound of formula (I) (as depicted above) wherein:

Ring A is phenyl, thienyl, pyridyl or thiazolyl;

R¹ is a substituent on carbon and is selected from 1-methyl-1-cyanoethyl, trifluoromethyl, chloro, methoxycarbonyl, 2-dimethylaminoethoxy, methoxy, hydroxy and 2-pyrrolidin-1-ylethoxy;

n is selected from 1-2; wherein the values of R¹ may be the same or different;

R² is hydrogen;

X is NR¹⁵ or O;

one of A, E, G and J is C which is attached to X of formula (I); the other three are all CR¹⁶ or two are CR¹⁶ and one is N;

R³ is hydrogen or methyl;

R⁴ is hydrogen, methyl, ethyl, 3-morpholinopropyl, cyclopropylmethyl, 2,2-dimethyl-1,3-dioxolan-4-ylmethyl, 2,3-dihydroxypropyl or 2-hydroxyethyl;

the bond [symbol] between the —NR⁵— and —CR³— of formula (I) is a double bond wherein R⁵ is absent;

R¹⁵ is selected from hydrogen, methyl or cyclopropylmethyl;

R¹⁶ is hydrogen;

or a pharmaceutically acceptable salt thereof.

Therefore in a further aspect of the invention there is provided a compound of formula (I) (as depicted above) wherein:

Ring A is phenyl, thien-2-yl, thien-3-yl, pyrid-2-yl, pyrid-3-yl, pyrid-4-yl, thiazol-4-yl, isoxazol-3-yl, 1,3-benzodioxol-5-yl, fur-2-yl, 1-methylpyrazol-3-yl, 1-methylpyrazol-5-yl, 1-t-butylpyrazol-5-yl, indol-5-yl, indol-6-yl, 2,3-dihydrobenzofuran-7-yl, imidazo[1,2-a]pyridin-2-yl or pyrimidin-4-yl;

R¹ is a substituent on carbon and is selected from fluoro, chloro, bromo, hydroxy, cyano, sulphamoyl, methyl, trifluoromethyl, cyclopropylaminomethyl, methylthiomethyl, mesylmethyl, dimethylaminomethyl, 1-(cyclopropyl)-1-hydroxymethyl, N-cyclopropyl-N-(t-butoxycarbonyl)aminomethyl, 1-methylpiperazin-4-ylmethyl, 1-hydroxy-1-cyclopropylethyl, 1-methyl-1-cyanoethyl, 2-methoxy-1,1-dimethylethyl, 1-carboxy-1-methylethyl, 1,1-difluoroethyl, 2-(dimethylamino)-1,1-dimethyl-2-oxoethyl, 3-(dimethylamino)propyl, 1,1-dimethylpropyl, t-butyl, methoxy, N-methylcarbamoylmethoxy, 2-(dimethylamino)ethoxy, 2-(pyrrolidin-1-yl)ethoxy, 2-(methoxy)ethoxy, 2-(1-methylpyrrolidin-2-yl)ethoxy, 2-(piperidin-1-yl)ethoxy, 2-(azepan-1-yl)ethoxy, 2-(morpholino)ethoxy, 3-(1-methylpiperazin-4-yl)propoxy, methoxycarbonyl, morpholinocarbonyl, N,N-dimethylsulphamoyl, N-(2,3-dihydroxypropyl)-N-methylsulphamoyl, N-(methyl)-N-(methoxy)sulphamoyl, 1-methylpiperidin-4-yloxy, N,N-dimethylcarbamoyl, cyclopropyl, piperidin-1-yl, morpholino, 1-cyclopropylethenyl, 3-(4-methylpiperazin-1-yl)prop-1-yn-1-yl, 3,3-dimethylbut-1-yn-1-yl, cyclopropylethynyl, 3-hydroxy-3-methylbut-1-yn-1-yl, 1,1-dimethylprop-2-yn-1-yl, 3-(dimethylamino)prop-1-yn-1-yl, mesyl, cyclopropylaminosulphonyl, azetidin-1-ylsulphonyl, morpholinosulphonyl, tetrahydrofur-2-ylmethylaminosulphonyl, 2-(hydroxymethyl)piperidin-1-ylsulphonyl, 3-(hydroxymethyl)piperidin-1-ylsulphonyl or 4-(hydroxymethyl)piperidin-1-ylsulphonyl;

n is selected from 0-2; wherein the values of R¹ may be the same or different;

R² is hydrogen;

X is NR¹⁵ or O;

one of A, E, G and J is C which is attached to X of formula (I); the other three are all CR¹⁶ or two are CR¹⁶ and one is N;
R³ is selected from hydrogen, methyl, N-(2-hydroxyethyl)amino, N,N-dimethylamino or methylthio;

R⁴ is selected from hydrogen, methyl, 1-methylpiperidin-3-ylmethyl, cyclopropylmethyl, 2,2-dimethyl-1,3-dioxolan-4-ylmethyl, piperidin-4-ylmethyl, 1-benzyloxycarbonylpipidin-4-ylmethyl, ethyl, 2-hydroxyethyl, 3-aminopropyl, 3-(t-butoxycarbonylamino)propyl, 3-morpholinopropyl, 2,3-dihydroxypropyl and cyclopropyl;

the bond [symbol] between the —NR⁵ and —CR³— of formula (I) is a double bond wherein R⁵ is absent; and

R¹⁵ is selected from hydrogen, methyl or cyclopropylmethyl;

R¹⁶ is hydrogen;

or a pharmaceutically acceptable salt thereof.

In another aspect of the invention, preferred compounds of the invention are any one of Examples 1, 55, 69, 80, 85, 90, 95, 100, 103 or 111 or a pharmaceutically acceptable salt thereof.

In another aspect of the invention, preferred compounds of the invention are any one of the Examples or a pharmaceutically acceptable salt thereof.

Another aspect of the present invention provides a process for preparing a compound of formula (I) or a pharmaceutically acceptable salt thereof which process (wherein variable are, unless otherwise specified, as defined in formula (I)) comprises of:

Process a) reacting an amine of the formula (II)

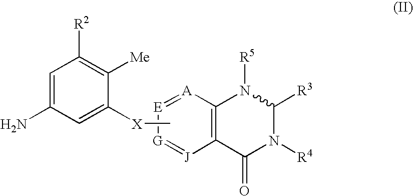

with an acid of formula (III):

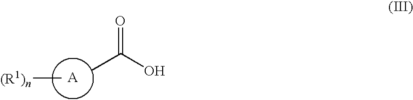

or an activated acid derivative thereof;
Process b) reacting a compound of formula (IV)

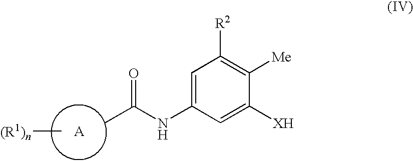

with an compound of formula (V):

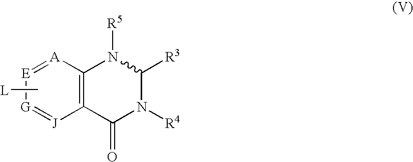

wherein L is a displaceable group;
Process c) reacting a compound of formula (VI) wherein L is a displaceable group:

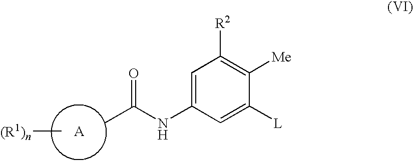

wherein L is a displaceable group; with an compound of formula (VII):

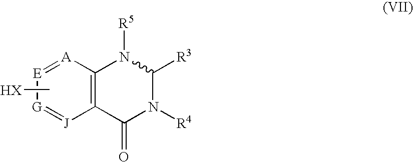

Process d) for compounds of formula (I) wherein R⁴ is not hydrogen; reacting a compound of formula (I) wherein R⁴ is hydrogen with a compound of formula (VIII):

R⁴-L (VIII)

wherein L is a displaceable group and R⁴ is not hydrogen;
Process e) for compounds of formula (I) wherein X is NR¹⁵ and R¹⁵ is —CH₂—C₂₋₆alkyl optionally substituted on carbon by one or more R²¹; reacting a compound of formula (I) wherein X is NR¹⁵ and R¹⁵ is hydrogen with a compound of formula (IX):

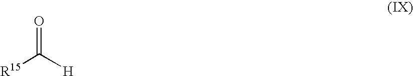

wherein R¹⁵ is C₁₋₅alkyl optionally substituted on carbon by one or more R²¹;
Process f) for compounds of formula (I) wherein X is NR¹⁵ and R¹⁵ is not hydrogen; reacting a compound of formula (I) wherein X is NR¹⁵ and R¹⁵ is hydrogen with a compound of formula (X):

R¹⁵-L (X)

wherein L is a displaceable group and R¹⁵ is not hydrogen;
and thereafter if necessary:
i) converting a compound of the formula (I) into another compound of the formula (I);
ii) removing any protecting groups;
iii) forming a pharmaceutically acceptable salt.

L is a displaceable group, suitable values for L are for example, a halo for example a chloro or bromo.

Specific reaction conditions for the above reactions are as follows.

Process a) Amines of formula (II) and acids of formula (III) may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Amines of formula (II) may be prepared according to Scheme 1:

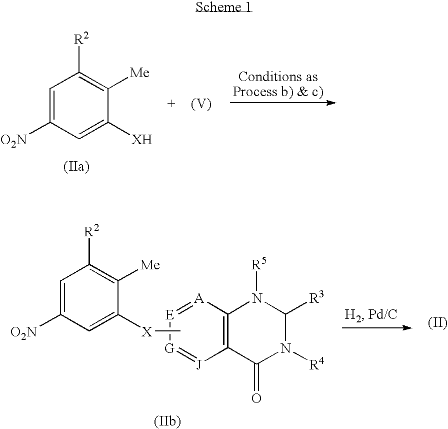

Compounds of formula (IIa) and (III) are commercially available compounds, or they are known in the literature or they may be prepared by standard processes known in the art.

Process b) and Process c) Compounds of formula (IV) and (V) and compounds of formula (VI) and (VII) can be reacted together by coupling chemistry utilizing an appropriate catalyst and ligand such as Pd₂(dba)₃ and BINAP respectively and a suitable base such as sodium tert-butoxide. The reaction usually requires thermal conditions often in the range of 80° C. to 100° C.

Compounds of formula (IV) may be prepared according to Scheme 2:

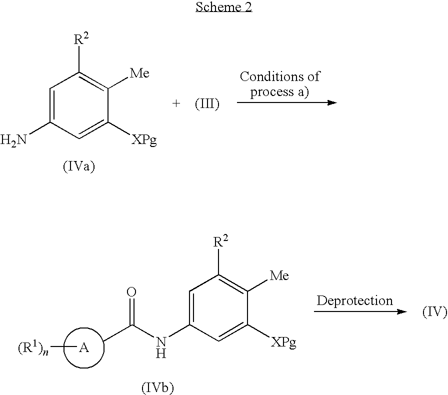

wherein Pg is a suitable protecting group.

Compounds of formula (VI) may be prepared according to Scheme 3:

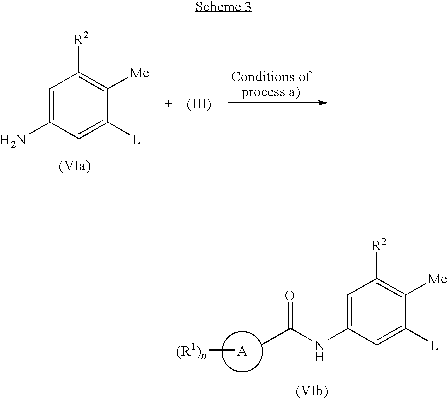

wherein Pg is a suitable protecting group.

Compounds of formula (IVa), (V), (VIa) and (VII) are commercially available compounds, or they are known in the literature or they may be prepared by standard processes known in the art.

Process d) Compounds of formula (I) and (VIII) can be reacted together in solvents such as DMF or CH₃CN in the presence of a base such as K₂CO₃ or Cs₂CO₃. The reaction usually requires thermal conditions in the range of 50° C. to 100° C.

Compounds of formula (VIII) are commercially available compounds, or they are known in the literature or they may be prepared by standard processes known in the art.

Process e) Compounds of formula (I) and (IX) can be reacted by standard reductive amination chemistry utilizing an appropriate solvent such as THF, dichloroethane or CH₃CN, in a pH range of 6-8 using a reducing agent such as sodium triacetoxyborohydride or sodium cyanoborohydride. The reaction is typically accomplished at 25° C. This reaction can also be achieved by utilizing formic acid. The reaction usually requires thermal conditions such as 70° C.

Compounds of formula (IX) are commercially available compounds, or they are known in the literature or they may be prepared by standard processes known in the art.

Process f) Compounds of formula (I) and (X) can be reacted together in various solvents such as DMF or CH₃CN in the presence of a base such as K₂CO₃ or Cs₂CO₃. The reaction usually requires thermal conditions in the range of 50° C. to 100° C.

Compounds of formula (X) are commercially available compounds, or they are known in the literature or they may be prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cancer activity which is believed to arise from the B-Raf inhibitory activity of the compound. These properties may be assessed, for example, using the procedure set out below:—

### B-Raf In Vitro ELISA Assay

Activity of human recombinant, purified wild type His-B-Raf protein kinase was determined in vitro using an enzyme-linked immunosorbent assay (ELISA) assay format, which measures phosphorylation of the B-Raf substrate, human recombinant, purified His-derived (detagged) MEK1. The reaction utilized 2.5 nM B-Raf, 0.15 μM MEK1 and 10 μM adenosine triphosphate (ATP) in 40 mM N-(2-hydroxyethyl)piperazine-N′-(2-ethanesulfonic acid hemisodium salt (HEPES), 5 mM 1,4-dithio-DL-threitol (DTT), 10 mM MgCl₂, 1 mM ethylenediaminetetraacetic acid (EDTA) and 0.2M NaCl (1×HEPES buffer), with or without compound at various concentrations, in a total reaction volume of 25 μl in 384 well plates. B-Raf and compound were preincubated in 1×HEPES buffer for 1 hour at 25° C. Reactions were initiated with addition of MEK1 and ATP in 1×HEPES buffer and incubated at 25° C. for 50 minutes and reactions stopped by addition of 10 μl 175 mM EDTA (final concentration 50 mM) in 1×HEPES buffer. 5 μl of the assay mix was then diluted 1:20 into 50 mM EDTA in 1×HEPES buffer, transferred to 384 well black high protein binding plates and incubated for 12 h at 4° C. Plates were washed in tris buffered saline containing 0.1% Tween20 (TBST), blocked with 50 μl Superblock (Pierce) for 1 hour at 25° C., washed in TBST, incubated with 50 μl rabbit polyclonal anti-phospho-MEK antibody (Cell Signaling) diluted 1:1000 in TBS for 2 h at 25° C., washed with TBST, incubated with 50 μl goat anti-rabbit horseradish peroxidase-linked antibody (Cell Signaling) diluted 1:2000 in TBS for 1 hour at 25° C. and washed with TBST. 50 μl of fluorogenic peroxidase substrate (Quantablu—Pierce) was added and following incubation for 45-60 mins, 50 μl QuantabluSTOP (Pierce) was added. Blue fluorescent product was detected at excitation 325 nm and emission 420 nm using a TECAN Ultra plate reader. Data was graphed and IC₅₀s calculated using Excel Fit (Microsoft).

When tested in the above in vitro assay, the compounds of the present invention exhibited activity less than 30 μM. For example the following results were obtained:

| Example No | IC₅₀ (μM) |
|---|---|
| 4 | 0.186 |
| 7 | 0.347 |
| 33 | 1.93 |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their B-Raf inhibitory properties. Accordingly the compounds of the present invention are expected to be useful in the treatment of diseases or medical conditions mediated alone or in part by B-Raf, i.e. the compounds may be used to produce a B-Raf inhibitory effect in a warm-blooded animal in need of such treatment.

Thus the compounds of the present invention provide a method for treating cancer characterised by inhibition of B-Raf, i.e. the compounds may be used to produce an anti-cancer effect mediated alone or in part by the inhibition of B-Raf.

Such a compound of the invention is expected to possess a wide range of anti-cancer properties as activating mutations in B-Raf have been observed in many human cancers, including but not limited to, melanoma, papillary thyroid tumours, cholangiocarcinomas, colon, ovarian and lung cancers. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In particular such compounds of the invention are expected to slow advantageously the growth of primary and recurrent solid tumours of, for example, the skin, colon, thyroid, lungs and ovaries. More particularly such compounds of the invention, or a pharmaceutically acceptable salt thereof, are expected to inhibit the growth of those primary and recurrent solid tumours which are associated with B-Raf, especially those tumours which are significantly dependent on B-Raf for their growth and spread, including for example, certain tumours of the skin, colon, thyroid, lungs and ovaries. Particularly the compounds of the present invention are useful in the treatment of melanomas.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore in the manufacture of a medicament for use in the production of a B-Raf inhibitory effect in a warm-blooded animal such as man.

According to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided the use of a compound of the formula (I), or a pharmaceutically acceptable salt thereof, as defined herein before in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (I), or a pharmaceutically acceptable salt thereof, as defined hereinbefore in the production of a B-Raf inhibitory effect in a warm-blooded animal such as man.

According to this aspect of the invention there is provided the use of a compound of the formula (I), or a pharmaceutically acceptable salt thereof, as defined hereinbefore in the production of an anti-cancer effect in a warm-blooded animal such as man.

According to a further feature of the invention, there is provided the use of a compound of the formula (I), or a pharmaceutically acceptable salt thereof, as defined herein before in the treatment of melanoma, papillary thyroid tumours; cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a B-Raf inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a B-Raf inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

The B-Raf inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
(x) cell cycle inhibitors including for example CDK inhibitors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors; and
(xi) endothelin antagonists, including endothelin A antagonists, endothelin B antagonists and endothelin A and B antagonists; for example ZD4054 and ZD1611 (WO 96 40681), atrasentan and YM598.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of B-Raf in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(x) where a synthesis is described as being analogous to that described in a previous example the amounts used are the millimolar ratio equivalents to those used in the previous example;
(xi) the following abbreviations have been used:

| | |
|---|---|
| HATU | O-(7-azabenzotriazol-1-yl)-N,N,N′,N′- |
| | tetramethyluronium |
| | hexafluorophosphate; |
| THF | tetrahydrofuran; |
| DMF | N,N-dimethylformamide; |
| EtOAc | ethyl acetate; |
| Pd₂(dba)₃ | tris(dibenzylideneacetone)dipalladium (0); |
| BINAP | (+/−)-2,2′-bis(diphenylphosphino)-1,1′-binaphthyl; |
| EDCI | 1-(3-dimethylaminopropyl)-3-ethylcarbodiimide |
| | hydrochloride; |
| HOBt | hydroxybenzotriazole; |
| TFA | trifluoroacetic acid; |
| DeoxoFluor ™ | 1,1′-[(trifluoro-λ⁴-sulfanyl)imino]bis(2-methoxyethane); |
| DCM | dichloromethane; and |
| DMSO | dimethylsulphoxide; |

(xii) “ISCO” refers to normal phase flash column chromatography using 12 g and 40 g pre-packed silica gel cartridges used according to the manufacturers instruction obtained from ISCO, Inc, 4700 superior street Lincoln, Nebr., USA.,
(xiii) “Reverse phase Gilson” or “Gilson HPLC” refers to a YMC-AQC18 reverse phase HPLC Column with dimension 20 mm/100 and 50 mm/250 in water/acetonitrile with 0.1% TFA as mobile phase, obtained from Waters Corporation 34, Maple street, Milford Mass., USA; and
(xiv) Parr Hydrogenator or Parr shaker type hydrogenators are systems for treating chemicals with hydrogen in the presence of a catalyst at pressures up to 5 atmospheres (60 psig) and temperatures to 80° C.

### Example 1

### 3-(1-Cyano-1-methylethyl)-N-{4-methyl-3-[(3-methyl-4-oxo-3,4-dihydroquinazolin-6-yl)amino]phenyl}benzamide

A stirred mixture of N-(3-amino-4-methylphenyl)-3-(1-cyano-1-methylethyl)benzamide (Method 87; 100 mg, 0.341 mmol), 6-bromo-3-methylquinazolin-4(3H)-one (Method 104; 82 mg, 0.341 mmol), sodium tert-butoxide (99 mg, 1.03 mmol), BINAP (21 mg, 0.034 mmol) in toluene (2 ml) was treated with Pd₂(dba)₃ (16 mg, 0.017 mmol). The reaction mixture was heated to 80° C. for 12 h. The reaction was then quenched with 10% NaOH(aq) and extracted with EtOAc. The organics were dried with NaCl(sat) and then Na₂SO₄(s) and removed under reduced pressure. The resulting solid was purified by column chromatography utilizing an ISCO system (10% MeOH in EtOAc) to give 91 mg (59%) of a light yellow solid. NMR: 10.28 (s, 1H), 8.57 (s, 1H), 8.00 (s, 1H), 7.90 (d, 1H), 7.78 (s, 1H), 7.72 (d, 2H), 7.44 (m, 3H), 7.25 (d, 1H), 3.46 (s, 3H), 2.17 (s, 3H), 1.72 (s, 6H); m/z 452.

### Examples 2-29

The following compounds were prepared by the procedure of Example 2, using the indicated starting materials.

| Ex | Compound | NMR | m/z | SM |
|---|---|---|---|---|
| 2 | 3-(1-Cyano-1-methylethyl)- | 10.29 (s, 1H), 8.34 (s, 1H), 8.20 (s, | 452 | Method |
| | N-{4-methyl-3- | 1H), 8.02 (s, 1H), 7.93 (m, 2H), | | 87 and |
| | [(3-methyl-4-oxo-3,4- | 7.80 (s, 1H), 7.74 (d, 1H), 7.59 (m, 1H), | | Method |
| | dihydroquinazolin-7- | 7.51 (s, 1H), 7.29 (d, 1H), 7.05 (s, | | 105 |
| | yl)amino]phenyl}- | 1H), 6.79 (s, 1H), 3.42 (s, 3H), | | |
| | benzamide | 2.19 (s, 3H), 1.74 (s, 6H) | | |
| 3 | 3-(1-Cyano-1-methylethyl)- | 10.23 (s, 1H), 8.03 (m, 2H), 7.99 (m, | 438 | Method |
| | N-{4-methyl-3- | 1H), 7.89 (d, 1H), 7.74 (s, 1H), | | 87 and |
| | [(4-oxo-3,4-dihydroquinazolin- | 7.72 (d, 1H), 7.56 (m, 2H), 7.43 (m, 2H), | | Method |
| | 6-yl)amino]- | 7.38 (d, 1H), 7.23 (d, 1H), 2.19 (s, | | 106 |
| | phenyl}benzamide | 3H), 1.74 (s, 6H) | | |
| 4 | 3-(1-Cyano-1-methylethyl)- | 11.94 (s, 1H), 10.21 (s, 1H), 7.99 (s, | 452 | Method |
| | N-{4-methyl-3- | 1H), 7.88 (d, 1H), 7.84 (s, 1H), | | 87 and |
| | [(2-methyl-4-oxo-3,4- | 7.71 (m, 2H), 7.56 (t, 1H), 7.40 (m, 4H), | | Method |
| | dihydroquinazolin-6- | 7.21 (d, 1H), 3.46 (s, 3H), 2.17 (s, | | 107 |
| | yl)amino]phenyl}- | 3H), 1.72 (s, 6H) | | |
| | benzamide | | | |
| 5 | N-{4-Methyl-3-[(3- | 10.37 (s, 1H), 8.23 (m, 2H), 8.14 (s, | 453 | Method |
| | methyl-4-oxo-3,4- | 1H), 7.99 (s, 1H), 7.94 (d, 1H), | | 88 and |
| | dihydroquinazolin-6- | 7.75 (m, 2H), 7.54 (d, 1H), 7.43 (m, 3H), | | Method |
| | yl)amino]phenyl}-3- | 7.24 (d, 1H), 3.44 (s, 3H), 2.18 (s, | | 104 |
| | (trifluoromethyl)- | 3H) | | |
| | benzamide | | | |
| 6 | 4-Chloro-N-{4-methyl-3- | 10.43 (s, 1H), 8.33 (s, 1H), 8.21 (d, | 488 | Method |
| | [(3-methyl-4-oxo-3,4- | 1H), 8.14 (s, 1H), 7.99 (s, 1H), | | 89 and |
| | dihydroquinazolin-6- | 7.89 (d, 1H), 7.72 (s, 1H), 7.54 (d, 1H), | | Method |
| | yl)amino]phenyl}-3- | 7.43 (m, 3H), 7.24 (d, 1H), 3.44 (s, | | 104 |
| | (trifluoromethyl)- | 3H), 2.18 (s, 3H) | | |
| | benzamide | | | |
| 7 | 3-(1-Cyano-1-methylethyl)- | 10.23 (s, 1H), 8.14 (s, 1H), 7.98 (m, | 565 | Method |
| | N-(4-methyl-3- | 2H), 7.89 (d, 1H), 7.72 (m, 2H), | | 87 and |
| | {[3-(3-morpholin-4- | 7.55 (m, 2H), 7.42 (m, 3H), 7.23 (d, | | Method |
| | ylpropyl)-4-oxo-3,4- | 1H), 3.95 (t, 2H), 3.47 (t, 4H), | | 116 |
| | dihydroquinazolin-6- | 2.27 (m, 6H), 2.17 (s, 3H), 1.82 (m, 2H), | | |
| | yl]amino}phenyl)- | 1.72 (s, 6H) | | |
| | benzamide | | | |
| 8 | 3-(1-Cyano-1-methylethyl)- | 10.22 (s, 1H), 8.16 (s, 1H), 7.98 (m, | 466 | Method |
| | N-{3-[(3-ethyl-4- | 2H), 7.89 (d, 1H), 7.72 (m, 2H), | | 87 and |
| | oxo-3,4-dihydroquinazolin- | 7.55 (m, 2H), 7.42 (m, 3H), 7.23 (d, | | Method |
| | 6-yl)amino]- | 1H), 3.95 (q, 2H), 2.17 (s, 3H), | | 117 |
| | 4-methylphenyl}- | 1.72 (s, 6H), 1.24 (t, 3H) | | |
| | benzamide | | | |
| 9 | 3-(1-Cyano-1- | 10.23 (s, 1H), 8.18 (s, 1H), 7.99 (s, | 492 | Method |
| | methylethyl)-N-(3-{[3- | 2H), 7.89 (d, 1H), 7.72 (m, 2H), | | 87 and |
| | (cyclopropylmethyl)-4- | 7.55 (m, 2H), 7.42 (m, 3H), 7.23 (d, | | Method |
| | oxo-3,4-dihydroquinazolin- | 1H), 3.78 (d, 2H), 2.17 (s, 3H), | | 118 |
| | 6-yl]amino}- | 1.72 (s, 6H), 1.22 (m, 1H), 0.46 (m, 2H), | | |
| | 4-methylphenyl)- | 0.39 (m, 2H) | | |
| | benzamide | | | |
| 10 | 3-(1-Cyano-1-methylethyl)- | 10.28 (m, 1H), 8.66 (s, 1H), 8.15 (s, | 453 | Method |
| | N-{4-methyl-3- | 1H), 8.02 (s, 1H), 7.93 (m, 2H), | | 87 and |
| | [(3-methyl-4-oxo-3,4- | 7.74 (d, 1H), 7.61 (m, 2H), 7.50 (m, 1H), | | Method |
| | dihydropyrido[3,4-d]- | 7.25 (d, 1H), 7.16 (s, 1H), 2.20 (s, | | 108 |
| | pyrimidin-6-yl)amino]- | 3H), 2.08 (s, 3H), 1.75 (s, 6H) | | |
| | phenyl}benzamide | | | |
| 11 | 3-(1-Cyano-1-methylethyl)- | 10.25 (s, 1H), 10.00 (s, br, 1H), | 538 | Method |
| | 5-[2- | 8.30 (s, br, 1H), 8.05 (s, br, 1H), 7.75 (s, | | 90 and |
| | (dimethylamino)ethoxy]- | 1H), 7.65 (s, 1H), 7.50 (m, 2H), | | Method |
| | N-{4-methyl-3-[(3- | 7.37 (m, 3H), 7.20 (m, 2H), 4.40 (t, 2H), | | 104 |
| | methyl-4-oxo-3,4- | 3.50 (t, 2H), 3.44 (s, 3H), 2.85 (s, | | |
| | dihydroquinazolin-6-yl)- | -6H), 2.15 (s, 3H), 1.70 (s, 6H) | | |
| | amino]phenyl}- | | | |
| | benzamide | | | |
| 12 | 3-(Cyano-dimethyl- | 10.30 (s, 1H), 8.39 (s, 1H), 8.20 (s, | 481 | Method |
| | methyl)-5-methoxy-N-[4- | br, 1H), 7.90 (s, 1H), 7.65 (m, 2H), | | 91 and |
| | methyl-3-(3-methyl-4- | 7.52 (m, 4H), 7.35 (m, 2H), 4.05 (s, | | Method |
| | oxo-3,4-dihydro- | 3H), 3.55 (s, 3H), 2.35 (s, 3H), | | 104 |
| | quinazolin-6-ylamino)- | 1.90 (s, 6H) | | |
| | phenyl]-benzamide | | | |
| 13 | 5-(1-Cyano-1-methylethyl)- | 10.17 (s, 1H), 8.14 (s, 1H), 7.98 (s, | 458 | Method |
| | N-{4-methyl-3- | 1H), 7.88 (d, 1H), 7.68 (d, 1H), | | 92 and |
| | [(3-methyl-4-oxo-3,4- | 7.54 (d, 1H), 7.43 (s, 2H), 7.39 (dd, 1H), | | Method |
| | dihydroquinazolin-6- | 7.28 (d, 1H), 7.22 (d, 1H), 3.45 (s, | | 104 |
| | yl)amino]phenyl}- | 3H), 2.16 (s, 3H), 1.77 (s, 6H) | | |
| | thiophene-2-carboxamide | | | |
| 14 | 6-(1-Cyano-1-methylethyl)- | 10.22 (s, 1H), 8.16 (m, 1H), 8.11 (d, | 453 | Method |
| | N-{4-methyl-3- | 1H), 8.05 (m, 2H), 7.84 (d, 1H), | | 94 and |
| | [(3-methyl-4-oxo-3,4- | 7.82 (d, 1H), 7.54 (d, 1H), 7.40 (m, | | Method |
| | dihydroquinazolin-6- | 3H), 7.28 (d, 1H), 3.43 (s, 3H), | | 104 |
| | yl)amino]phenyl}- | 2.17 (s, 3H), 1.79 (s, 6H) | | |
| | pyridine-2-carboxamide | | | |
| 15 | 4-(1-Cyano-1- | 10.20 (s, 1H), 8.22 (s, 1H), 8.14 (d, | 458 | Method |
| | methylethyl)-N-{4- | 1H), 8.01 (s, 1H), 7.82 (d, 1H), | | 93 and |
| | methyl-3-[(3-methyl-4- | 7.69 (d, 1H), 7.57 (d, 1H), 7.44 (s, 1H), | | Method |
| | oxo-3,4-dihydroquinazolin- | 7.38 (m, 2H), 7.24 (s, 1H) 3.45 (s, | | 104 |
| | 6-yl)amino]- | 3H), 2.17 (s, 3H), 1.69 (s, 6H) | | |
| | phenyl}thiophene-2- | | | |
| | carboxamide | | | |
| 16 | 4-Chloro-3-(1-cyano-1- | 10.24 (s, 1H), 8.11 (s, 1H), 7.91 (m, | 486 | Method |
| | methylethyl)-N-{4- | 3H), 7.65 (d, 2H), 7.48 (dd, 1H), | | 95 and |
| | methyl-3-[(3-methyl-4- | 7.37 (s, 2H), 7.34 (m, 1H), 7.17 (d, | | Method |
| | oxo-3,4-dihydroquinazolin- | 1H), 3.39 (s, 3H), 2.12 (s, 3H), | | 104 |
| | 6-yl)amino]- | 1.78 (s, 6H) | | |
| | phenyl}benzamide | | | |
| 17 | 3-(1-Cyano-1-methylethyl)- | 10.22 (s, 1H), 8.06 (s, 1H), 8.00 (m, | 552 | Method |
| | N-[3-({3-[(2,2- | 2H), 7.89 (d, 1H), 7.72 (m, 2H), | | 87 and |
| | dimethyl-1,3-dioxolan-4- | 7.55 (m, 2H), 7.44 (m, 3H), 7.23 (d, | | Method |
| | yl)methyl]-4-oxo-3,4- | 1H), 4.37 (m, 1H), 4.15 (dd, 1H), | | 124 |
| | dihydroquinazolin-6- | 4.00 (m, 2H), 3.70 (dd, 1H), 2.17 (s, | | |
| | yl}amino)-4- | 3H), 1.72 (s, 6H), 1.31 (s, 3H), | | |
| | methylphenyl]benzamide | 1.21 (s, 3H) | | |
| 18 | 3-(Cyano-dimethyl- | 10.20 (s, 1H), 8.35 (s, 1H), 8.15 (s, | 538 | Method |
| | methyl)-5-methyl- | br, 1H), 7.75 (s, 1H), 7.65 (s, 1H), | | 96 and |
| | carbamoylmethoxy-N-[4- | 7.60 (d, 1H), 7.45 (m, 4H), 7.35 (s, | | Method |
| | methyl-3-(3-methyl-4- | 1H), 7.26 (d, 1H), 4.60 (s, 2H), | | 104 |
| | oxo-3,4-dihydro- | 3.50 (s, 3H), 2.70 (d, 3H), 2.20 (s, 3H), | | |
| | quinazolin-6-ylamino)- | 1.72 (s, 6H) | | |
| | phenyl]-benzamide | | | |
| 19 | 3-(1-Cyano-1-methylethyl)- | 11.25 (s, 1H), 10.40 (s, 1H), 8.70 (s, | 580 | Method |
| | N-{4-methyl-3- | 1H), 8.30 (s, br, 1H), 7.85 (s, 1H), | | 97 and |
| | [(3-methyl-4-oxo-3,4- | 7.70 (m, 2H), 7.60 (s, 1H), 7.45 (m, | | Method |
| | dihydroquinazolin-6- | 3H), 7.30 (m, 2H), 4.60 (m, 2H), | | 104 |
| | yl)amino]phenyl}-5-(2- | 3.98 (m, 4H), 3.55 (m, 4H), 3.25 (m, | | |
| | morpholin-4-ylethoxy)- | 2H), 3.20 (s, 3H), 2.20 (s, 3H), | | |
| | benzamide | 1.75 (s, 6H). | | |
| 20 | 3-(Cyano-dimethyl- | 10.10 (s, 1H), 6.30 (s, br, 1H), | 578 | Method |
| | methyl)-N-[4-methyl-(3- | 8.10 (s, 1H), 7.90 (s, br, 1H), 7.60 (s, | | 98 and |
| | methyl-4-oxo-3,4- | 1H), 7.55 (s, 1H), 7.45 (d, 1H), | | Method |
| | dihydro-quinazolin-6- | 7.40 (s, 1H), 7.30 (m, 3H), 7.15 (m, 2H), | | 104 |
| | ylamino)-phenyl]-5-(2- | 4.30 (m, 2H), 3.40 (m, 4H), 3.30 (s, | | |
| | piperidin-1-yl-ethoxy)- | 3H), 2.90 (m, 2H), 2.10 (s, 3H), | | |
| | benzamide | 1.70 (m, 2H), 1.60 (m, 9H), 1.30 (m, 1H). | | |
| 21 | 3-(Cyano-dimethyl- | 10.25 (s, 1H), 8.30 (s, 1H), 8.06 (s, | 607 | Method |
| | methyl)-N-[4-methyl-3- | br, 1H), 7.80 (s, 1H), 7.62 (s, 1H), | | 99 and |
| | (3-methyl-4-oxo-3,4- | 7.56 (d, 1H), 7.40 (m, 4H), 7.20 (m, | | Method |
| | dihydro-quinazolin-6- | 2H), 4.20 (m, 2H), 3.30 (m, 6H), | | 104 |
| | ylamino)-phenyl]-5-[3- | 3.20 (s, 3H), 2.85 (s, 3H), 2.45 (m, | | |
| | (4-methyl-piperazin-1- | 4H), 2.20 (m, 5H), 1.75 (s, 6H). | | |
| | yl)-propoxy]-benzamide | | | |
| 22 | 3-(Cyano-dimethyl- | 10.05 (s, 1H), 8.30 (s, 1H), 7.50 (s, | 578 | Method |
| | methyl)-N-[4-methyl-3- | 1H), 7.40 (m, 2H), 7.25 (m, 4H), | | 100 and |
| | (3-methyl-4-oxo-3,4- | 7.00 (m, 2H), 3.25 (m, 7H), 2.80 (m, | | Method |
| | dihydro-quinazolin-6- | 2H), 2.60 (s, 3H), 2.10 (m, 1H), | | 104 |
| | ylamino)-phenyl]-5-[2- | 2.00 (s, 3H), 1.70 (m, 2H), 1.50 (m, 8H) | | |
| | (1-methyl-pyrrolidin-2- | | | |
| | yl)-ethoxy]-benzamide | | | |
| 23 | 3-(2-Azepan-1-yl- | 10.35 (m, 2H), 8.45 (s, 1H), 8.20 (s, | 592 | Method |
| | ethoxy)-5-(cyano- | br, 1H), 7.80 (s, 1H), 7.75 (s, 1H), | | 101 and |
| | dimethyl-methyl)-N-[4- | 7.65 (m, 2H), 7.50 (m, 3H), 7.35 (m, | | Method |
| | methyl-3-(3-methyl-4- | 2H), 4.55 (t, 2H), 3.50 (m, 2H), | | 104 |
| | oxo-3,4-dihydro- | 3.45 (s, 3H), 3.29 (m, 4H), 2.25 (s, 3H), | | |
| | quinazolin-6-ylamino)- | 1.60-1.90 (m, 14H) | | |
| | phenyl]-benzamide | | | |
| 24 | 3-(Cyano-dimethyl- | 10.20 (s, 1H), 8.37 (s, 1H), 8.15 (s, | 525 | Method |
| | methyl)-5-(2-methoxy- | br, 1H), 7.82 (s, 1H), 7.65 (m, 2H), | | 102 and |
| | ethoxy)-N-[4-methyl-3- | 7.50 (m, 4H), 7.30 (m, 2h), 4.22 (t, | | Method |
| | (3-methyl-4-oxo-3,4- | 2H), 3.75 (t, 2H), 3.50 (s, 3H), | | 104 |
| | dihydro-quinazolin-6- | 3.36 (s, 3H), 2.23 (s, 3H), 1.78 (s, 6H) | | |
| | ylamino)-phenyl]- | | | |
| | benzamide | | | |
| 25 | 3-(Cyano-dimethyl- | 10.41-10.33 (m, 2h), 8.44 (s, 1H), | 564 | Method |
| | methyl)-N-[4-methyl-3- | 8.19 (s, br, 1H), 7.83 (s, 1H), | | 103 and |
| | (3-methyl-4-oxo-3,4- | 7.69-7.33 (m, 8H), 3.45 (s, 3H), | | Method |
| | dihydro-quinazolin-6- | 3.40-3.14 (m, 4H), 2.86 (m, 3h), 2.34-2.14 (m, | | 104 |
| | ylamino)-phenyl]-5-(1- | 7H), 1.95 (m, 1H), 1.79 (s, 6H) | | |
| | methyl-piperidin-4- | | | |
| | yloxy)-benzamide | | | |
| 26 | tert-Butyl {3-[6-[(5-{[3- | | 595 | Method |
| | (1-cyano-1-methylethyl)- | | | 87 and |
| | benzoyl]amino}-2- | | | Method |
| | methylphenyl)amino]-4- | | | 123 |
| | oxoquinazolin-3(4H)- | | | |
| | yl]propyl}carbamate | | | |
| 27 | 3-(1-Cyano-1-methylethyl)- | 10.29 (s, 1H), 8.25 (m, 1H), 8.00 (m, | 586 | Method |
| | N-[4-methyl-3- | 1H), 7.90 (d, 1H), 7.78 (m, 1H), | | 87 and |
| | ({3-[(1-methylpiperidin- | 7.72 (d, 2H), 7.56 (m, 2H), 7.41 (m, | | Method |
| | 3-yl)methyl]-4-oxo-3,4- | 3H), 7.24 (d, 2H), 2.67 (m, 3H), | | 121 |
| | dihydroquinazolin-6- | 2.17 (s, 3H), 3.44 (s, 3H), 1.80 (m, | | |
| | yl}amino)phenyl]- | 4H), 1.72 (s, 6H), 1.22 (m, 2H) | | |
| | benzamide | | | |
| 28 | Benzyl 4-{[6-[(5-{[3-(1- | | 669 | Method |
| | cyano-1-methylethyl)- | | | 87 and |
| | benzoyl]amino}-2- | | | Method |
| | methylphenyl)amino]-4- | | | 122 |
| | oxoquinazolin-3(4H)- | | | |
| | yl]methyl}piperidine-1- | | | |
| | carboxylate | | | |
| 29 | 3-(1-Cyano-1- | 10.23 (s, 1H), 8.03 (m, 1H), 7.98 (m, | 478 | Method |
| | methylethyl)-N-{3-[(3- | 2H), 7.89 (d, 1H), 7.72 (m, 2H), | | 87 and |
| | cyclopropyl-4-oxo-3,4- | 7.56 (t, 1H), 7.51 (d, 1H), 7.41 (m, | | Method |
| | dihydroquinazolin-6- | 3H), 7.24 (d, 2H), 3.19 (m, 1H), | | 110 |
| | yl)amino]-4-methyl- | 2.17 (s, 3H), 1.72 (s, 6H), 0.99 (m, | | |
| | phenyl}-benzamide | 2H), 0.90 (m, 2H) | | |

### Example 30

### 3-(1-Cyano-1-methylethyl)-N-(3-{[3-(2,3-dihydroxypropyl)-4-oxo-3,4-dihydroquinazolin-6-yl]amino}-4-methylphenyl)benzamide

A stirred mixture of 3-(1-cyano-1-methylethyl)-N-[3-({3-[(2,2-dimethyl-1,3-dioxolan-4-yl)methyl]-4-oxo-3,4-dihydroquinazolin-6-yl}amino)-4-methylphenyl]benzamide (Example 17; 129 mg, 0.440 mmol) in THF (3 ml) was treated with 3 M HCl (3 ml) at 25° C. for 30 min. The reaction mixture was quenched with 10% NaOH(aq) and extracted with EtOAc. The organics were dried with NaCl(sat) and then Na₂SO₄(s) and removed under reduced pressure to provide 107 mg (86%) of a white solid. NMR: 10.22 (s, 1H), 7.98 (m, 3H), 7.89 (d, 1H), 7.72 (m, 2H), 7.55 (m, 2H), 7.43 (m, 3H), 7.23 (d, 1H), 4.99 (d, 1H), 4.72 (t, 1H), 4.23 (dd, 1H), 3.74 (m, 1H), 3.63 (dd, 1H), 3.38 (m, 2H), 2.17 (s, 3H), 1.72 (s, 6H); m/z 512.

### Example 31

### 3-(1-Cyano-1-methylethyl)-N-(3-{[3-(2-hydroxyethyl)-4-oxo-3,4-dihydroquinazolin-6-yl]amino}-4-methylphenyl)benzamide

A stirred mixture of N-(3-amino-4-methylphenyl)-3-(1-cyano-1-methylethyl)benzamide (Method 87, 129 mg, 0.440 mmol), 6-bromo-3-(2-{[tert-butyl(dimethyl)silyl]oxy}ethyl)quinazolin-4(3H)-one (Method 120; 150 mg, 0.441 mmol), sodium tert-butoxide (127 mg, 1.32 mmol), BINAP (27 mg, 0.044 mmol) in toluene (3 ml) was treated with Pd₂(dba)₃ (20 mg, 0.022 mmol). The reaction mixture was heated to 80° C. for 12 h. The reaction was then quenched with 10% NaOH(aq) and extracted with EtOAc. The organics were dried with NaCl(sat) and then Na₂SO₄(s). The organics were removed under reduced pressure and the resulting solid was treated with 6 M HCl (5 ml) and stirred for 5 min at 25° C. The reaction was then quenched with 10% NaOH(aq) and extracted with EtOAc. The organics were dried with NaCl(sat) and then Na₂SO₄(s) and removed under reduced pressure. The resulting solid was purified by column chromatography utilizing an ISCO system (10% MeOH in EtOAc) to give 125 mg (59%) of a light yellow solid. NMR: 10.23 (s, 1H), 8.03 (s, 1H), 7.98 (m, 2H), 7.89 (d, 1H), 7.72 (m, 2H), 7.55 (m, 2H), 7.42 (m, 3H), 7.23 (d, 1H), 4.91 (t, 1H), 3.97 (t, 2H), 3.62 (q, 2H), 2.17 (s, 3H), 1.72 (s, 6H); m/z 482.

### Example 32

### 3-(Cyano-dimethyl-methyl)-N-{4-methyl-3-[cyclopropylmethyl-(3-methyl-4-oxo-3,4-dihydro-quinazolin-6-yl)-amino]-phenyl}-benzamide

A solution of 3-(1-cyano-1-methylethyl)-N-{4-methyl-3-[(3-methyl-4-oxo-3,4-dihydroquinazolin-6-yl)amino]phenyl}benzamide (Example 1; 100 mg, 0.22 mmol) and 1 ml of cyclopropanecarbaldehyde in 1 ml of formic acid was stirred at 70° C. for 12 h. 6N HCl(aq) (5 ml) was then added to the mixture. The solution was extracted with ether. The pH of the aqueous layer was then adjusted to pH12 with 10% NaOH(aq) and extracted with DCM (3×30 ml). The organics were removed under reduced pressure and the resulting solid was purified by column chromatography utilizing an ISCO system (DCM-methanol-ethylamine), then by Gilson (0.1% TFA in acetonitrile and water) to give 37 mg of light yellow solid (33.3%). NMR: 10.15 (s, 1H), 7.90 (s, 1H), 7.80 (s, 1H), 7.75 (d, 1H), 7.55 (m, 3H), 7.40 (t, 1H), 7.30 (d, 1H), 7.20 (d, 1H), 6.95 (d, 1H), 6.85 (d, 1H), 3.42 (d, 1H), 3.20 (s, 3H), 1.90 (s, 3H), 1.55 (s, 6H), 0.90 (m, 1H), 0.26 (m, 2H), 0.05 (m, 2H); m/z 505.

### Example 33

The following compound was prepared by the procedure of Example 32, using 3-(1-cyano-1-methylethyl)-N-{4-methyl-3-[(3-methyl-4-oxo-3,4-dihydroquinazolin-6-yl)amino]phenyl}benzamide (Example 1) and the indicated starting material.

| Ex. | Compound | NMR | m/z | SM |
|---|---|---|---|---|
| 33 | 3-(Cyano-dimethyl- | 10.29 (s, 1H), 8.20 (s, 1H), | 465 | formalaldehyde |
| | methyl)-N-{4- | 7.98 (s, 1H), 7.85 (d, 1H), | | |
| | methyl-3-[methyl-(3- | 7.45-7.79 (m, 5H), 7.30 (s, 1h), | | |
| | methyl-4-oxo-3,4- | 7.05 (s, 1H), 6.95 (d, 1H), | | |
| | dihydro-quinazolin-6- | 3.50 (s, 3H), 3.20 (s, 3H), 2.00 (s, | | |
| | yl)-amino]-phenyl}- | 3H), 1.62 (s, 6H) | | |
| | beazamide | | | |

### Example 34

### 3-(1-Cyano-1-methylethyl)-5-hydroxy-N-{4-methyl-3-[(3-methyl-4-oxo-3,4-dihydroquinazolin-6-yl)amino]phenyl}benzamide

A solution of 3-(cyano-dimethyl-methyl)-5-methoxy-N-[4-methyl-3-(3-methyl-4-oxo-3,4-dihydro-quinazolin-6-ylamino)-phenyl]-benzamide (Example 12) in 1M BBr₃ in DCM was stirred at 25° C. for 4 h. Crushed ice was then added to the mixture slowly. The pH of the resulting solution was adjusted to pH12 with 1N NaOH(aq) and the organic layer was separated and discarded. The water layer was then acidified with 10% HCl(aq) to pH 6˜7 and the fine dark red solid was collected by vacuum filtration. Purification utilizing a reverse phase Gilson (0.1% TFA in acetonitrile and water) provided 210 mg of light yellow solid (27% for two steps) as desired product. NMR: δ 10.10 (s, 1H), 9.95 (s, br, 1H), 8.20 (s, 1H), 8.00 (s, br, 1H), 7.70 (s, 1H), 7.50 (d, 1H), 7.36 9 m, 4H), 7.20 (m, 2H), 7.05 (s, 1H), 3.45 (s, 3H), 2.10 (s, 3H), 1.62 (s, 6H); m/z 467.

### Example 35

### 3-(1-Cyano-1-methylethyl)-N-{4-methyl-3-[(3-methyl-4-oxo-3,4-dihydroquinazolin-6-yl)amino]-phenyl}-5-(2-pyrrolidin-1-ylethoxy)benzamide

A suspension of 3-(1-cyano-1-methylethyl)-5-hydroxy-N-{4-methyl-3-[(3-methyl-4-oxo-3,4-dihydroquinazolin-6-yl)amino]phenyl}benzamide (Example 34; 120 mg, 0.257 mmol), 1-(2-chloro-ethyl)-pyrrolidine hydrochloride (52 mg, 0.308 mmol), K₂CO₃ (355 mg, 2.57 mmol) and sodium iodide (4 mg, 0.0257 mmol) in acetone (10 ml) was heated to reflux for 4 h. The salt was removed by filtration and washed with acetone. The filtrate was concentrated under reduced pressure and the residue was purified by a Gilson HPLC (0.1% TFA in acetonitrile and water) to provide 55 mg of a light yellow solid (38%). NMR: δ 10.30 (s, 1H), 9.95 (s, br, 1H), 8.25 (s, 1H), 8.05 (s, br, 1H), 7.80 (s, 1H), 7.70 (s, 1H), 7.45 (m, 5H), 7.30 (m, 2H), 4.43 (m, 2H), 3.50 (m, 4H), 3.15 (m, 2H), 2.25 (s, 3H), 2.10 (m, 2H), 1.95 (m, 2H), 1.80 (s, 6H); m/z 564.

### Example 36

### 3-(1-Cyano-1-methylethyl)-N-{4-methyl-3-[(3-methyl-4-oxo-3,4-dihydroquinazolin-6-yl)oxy]phenyl}benzamide

A solution of 6-(5-amino-2-methylphenoxy)-3-methylquinazolin-4(3H)-one (Method 109; 150 mg, 0.471 mmol), 3-(1-cyano-1-methylethyl)benzoic acid (Method 40; 89 mg, 0.471 mmol) and diisopropylethylamine (246 μL, 1.41 mmol, 3.0 equiv) in 2 ml of DMF was treated with HATU (215 mg, 0.565 mmol, 1.2 equiv). The reaction stirred at 50° C. for 12 h. The reaction was quenched with H₂O and extracted with EtOAc. The organics were dried with NaCl(sat) and then Na₂SO₄(s) and removed under reduced pressure. The resulting solid was purified by column chromatography utilizing an ISCO system (EtOAc-hexane, 4:1) to give 114 mg of light yellow solid (45%). NMR: 10.34 (s, 1H), 8.30 (s, 1H), 7.97 (s, 1H), 7.88 (d, 1H), 7.73 (m, 2H), 7.56 (m, 4H), 7.36 (m, 2H), 3.45 (s, 3H), 2.14 (s, 3H), 1.71 (s, 6H); m/z 553.

### Examples 37-103

The following compounds were prepared by the procedure of Example 36, using 6-(5-amino-2-methylphenoxy)-3-methylquinazolin-4(3H)-one (Method 109) or 6-[(5-amino-2-methylphenyl)amino]-3-methylquinazolin-4(3H)-one (Method 232) and the appropriate starting materials. Compounds were purified by column chromatography using reverse or normal phase chromatography.

| Ex. | Compound | NMR | m/z | SM |
|---|---|---|---|---|
| 37 | Methyl 3-[({4-methyl-3- | 10.49 (s, 1H), 8.47 (s, 1H), 8.31 (s, | 444 | Method 109 |
| | [(3-methyl-4-oxo-3,4- | 1H), 8.16 (d, 1H), 8.14 (d, 1H), | | and 3- |
| | dihydro quinazolin-6- | 7.75 (d, 1H), 7.66 (d, 1H), 7.61 (d, | | (methoxy- |
| | yl)oxy] phenyl}amino)- | 1H), 7.55 (m, 2H), 7.35 (m, 2H), | | carbonyl)- |
| | carbonyl]benzoate | 3.88 (s, 3H), 3.45 (s, 3H), 2.14 (s, | | benzoic acid |
| | | 3H) | | |
| 38 | 5-(1-Cyano-1-methylethyl)- | 10.30 (s, 1H), 8.31 (s, 1H), 8.20 (s, | 459 | Method 109 |
| | N-{4-methyl-3- | 1H), 7.88 (s, 1H), 7.72 (d, 1H), | | and Method |
| | [(3-methyl-4-oxo-3,4- | 7.56 (m, 2H), 7.49 (s, 1H), | | 144 |
| | dihydro quinazolin-6- | 7.35 (m, 1H), 7.26 (s, 1H), 3.45 (s, 3H), | | |
| | yl)oxy]phenyl}thiophene- | 2.13 (s, 3H), 1.76 (s, 6H) | | |
| | 2-carboxamide | | | |
| 39 | 2-(1-Cyano-1-methylethyl)- | 10.15 (s, 1H), 8.45 (s, 1H), 8.31 (s, | 460 | Method 109 |
| | N-{4-methyl-3- | 1H), 7.74 (d, 1H), 7.65 (dd, 1H), | | and Method |
| | [(3-methyl-4-oxo-3,4- | 7.58 (d, 1H), 7.54 (d, 1H), 7.38 (s, | | 47 |
| | dihydro quinazolin-6- | 1H), 7.34 (s. 1H), 3.45 (s, 3H), | | |
| | yl)oxy]phenyl}-1,3- | 2.14 (s, 3H), 1.87 (s, 6H) | | |
| | thiazole-4-carboxamide | | | |
| 40 | 4-Chloro-N-{4-methyl-3- | 10.53 (s, 1H), 8.32 (m, 2H), | 489 | Method 109 |
| | [(3-methyl-4-oxo-3,4- | 8.20 (d, 1H), 7.89 (d, 1H), 7.74 (d, 1H), | | and 4- |
| | dihydroquinazolin-6- | 7.56 (m, 3H), 7.37 (m, 2H), | | chloro-3- |
| | yl)oxy]phenyl}-3- | 3.45 (s, 3H), 2.15 (s, 3H) | | (trifluoromethyl)- |
| | (trifluoromethyl)- | | | benzoic acid |
| | benzamide | | | |
| 41 | 2-Chloro-N-{4-methyl-3- | 10.48 (s, 1H), 8.02-8.15 (m, 4H), | 487 | Method 232 |
| | [(3-methyl-4-oxo-3,4- | 7.94 (d, 1H), 7.75 (s, 1H), | | and 2- |
| | dihydroquinazolin-6- | 7.50-7.56 (m, 1H), 7.40-7.47 (m, 3H), | | Chloro-5- |
| | yl)amino]phenyl}-5- | 7.24 (d, 1H), 3.43 (s, 3H), 2.17 (s, | | (trifluoromethyl)- |
| | (trifluoromethyl)- | 3H) | | benzoic |
| | benzamide | | | acid |
| 42 | 2-Fluoro-N-{4-methyl-3- | 10.52 (s, 1H), 8.14 (s, 1H), | 471 | Method 232 |
| | [(3-methyl-4-oxo-3,4- | 7.92-8.06 (m, 3H), 7.66 (s, 1H), 7.57 (d, | | and 2- |
| | dihydroquinazolin-6- | 2H), 7.52 (s, 1H), 7.32-7.46 (m, | | Fluoro-5- |
| | yl)amino]phenyl}-5- | 4H), 7.23 (d, 1H), 3.43 (s, 3H), | | (trifluoromethyl)- |
| | (trifluoromethyl)- | 2.16 (s, 4H) | | benzoic |
| | benzamide | | | acid |
| 43 | 3-Fluoro-N-{4-methyl-3- | 10.47 (s, 1H), 8.01-8.14 (m, 4H), | 471 | Method 232 |
| | [(3-methyl-4-oxo-3,4- | 7.93 (d, 1H), 7.74 (s, 1H), | | and 3- |
| | dihydroquinazolin-6- | 7.49-7.55 (m, 1H), 7.37-7.47 (m, 3H), | | Fluoro-5- |
| | yl)amino]phenyl}-5- | 7.23 (d, 1H), 3.44 (s, 3H), 2.17 (s, | | (trifluoromethyl)- |
| | (trifluoromethyl)- | 3H) | | benzoic |
| | benzamide | | | acid |
| 44 | 4-Fluoro-N-{4-methyl-3- | 10.41 (s, 1H), 8.31 (s, 2H), 8.13 (s, | 471 | Method 232 |
| | [(3-methyl-4-oxo-3,4- | 1H), 8.01 (s, 1H), 7.69 (s, 2H), | | and 4- |
| | dihydroquinazolin-6- | 7.53 (s, 1H), 7.42 (s, 3H), 7.23 (s, | | Fluoro-3- |
| | yl)amino]phenyl}-3- | 1H), 3.44 (s, 3H), 2.18 (s, 3H) | | (trifluoromethyl)- |
| | (trifluoromethyl)- | | | benzoic |
| | benzamide | | | acid |
| 45 | 1-Methyl-N-{4-methyl- | 10.33 (s, 1H), 8.14 (s, 1H), 8.01 (s, | 457 | Method 232 |
| | 3-[(3-methyl-4-oxo-3,4- | 1H), 7.67 (s, 1H), 7.48-7.56 (m, | | and 1- |
| | dihydroquinazolin-6- | 2H), 7.39-7.45 (m, 3H), 7.23 (d, | | Methyl-3- |
| | yl)amino]phenyl}-3- | 1H), 4.12 (s, 3H), 3.44 (s, 3H), | | (trifluoromethyl)- |
| | (trifluoromethyl)-1H- | 2.17 (s, 3H) | | 1H- |
| | pyrazole-5-carboxamide | | | pyrazole-5- |
| | | | | carboxylic |
| | | | | acid |
| 46 | 1-tert-Butyl-3-methyl-N- | 10.47 (s, 1H), 8.79 (s, 1H), | 445 | Method 232 |
| | {4-methyl-3-[(3-methyl- | 7.56-7.70 (m, 3H), 7.41 (dd, 1H), | | and 1-tert- |
| | 4-oxo-3,4-dihydro- | 7.26-7.35 (m, 2H), 7.17 (d, 1H), 3.45 (s, | | Butyl-3- |
| | quinazolin-6- | 3H), 2.10 (s, 3H), 2.08 (s, 3H), | | methyl-1H- |
| | yl)amino]phenyl}-1H- | 1.50 (s, 9H) | | pyrazole-5- |
| | pyrazole-5-carboxamide | | | carboxylic |
| | | | | acid |
| 47 | tert-Butyl cyclopropyl[4- | | 622 | Method 232 |
| | [({4-methyl-3-[(3- | | | and Method |
| | methyl-4-oxo-3,4- | | | 48 |
| | dihydroquinazolin-6- | | | |
| | yl)amino]phenyl}amino)- | | | |
| | carbonyl]-2- | | | |
| | (trifluoromethyl)benzyl]- | | | |
| | carbamate | | | |
| 48 | 3-(3,3-Dimethylbut-1- | 10.22 (s, 1H), 8.16 (s, 1H), 7.95 (s, | 465 | Method 232 |
| | yn-1-yl)-N-{4-methyl-3- | 1H), 7.88 (s, 1H), 7.83 (d, 1H), | | and Method |
| | [(3-methyl-4-oxo-3,4- | 7.74 (d, 1H), 7.55-7.38 (m, 6H), | | 49 |
| | dihydroquinazolin-6- | 7.20 (d, 1H), 3.44 (s, 3H), 2.16 (s, | | |
| | yl)amino]phenyl}- | 3H), 1.19 (s, 9H) | | |
| | benzamide | | | |
| 49 | 3-(3-Hydroxy-3- | 10.20 (s, 1H), 8.15 (s, 1H), 7.96 (s, | 467 | Method 232 |
| | methylbut-1-yn-1-yl)-N- | 1H), 7.88 (s, 1H), 7.85 (d, 1H), | | and Method |
| | {4-methyl-3-[(3-methyl- | 7.73 (d, 1H), 7.55-7.50 (m, 3H), | | 50 |
| | 4-oxo-3,4-dihydro- | 7.48-7.38 (m, 3H), 7.20 (d, 1H), | | |
| | quinazolin-6-yl)amino]- | 3.43 (s, 3H), 2.16 (s, 3H), 1.49 (s, | | |
| | phenyl}benzamide | 6H) | | |
| 50 | 3-(Cyclopropylethynyl)- | 10.19 (s, 1H), 8.14 (s, 1H), 7.98 (s, | 449 | Method 232 |
| | N-{4-methyl-3-[(3- | 1H), 7.90 (s, 1H), 7.83 (d, 1H), | | and Method |
| | methyl-4-oxo-3,4- | 7.75 (d, 1H), 7.55-7.39 (m, 6H), | | 51 |
| | dihydroquinazolin-6- | 7.20 (d, 1H), 3.44 (s, 3H), 2.16 (s, | | |
| | yl)amino]phenyl}- | 3H), 1.58-1.51 (m, 1H), | | |
| | benzamide | 0.92-0.87 (m, 2H), 0.77-0.73 (m, 2H) | | |
| 51 | 5-(1-Cyano-1- | 10.53 (s, 1H), 9.08 (d, 1H), | 453 | Method 232 |
| | methylethyl)-N-{4- | 8.94 (d, 1H), 8.59 (s, 1H), 8.39 (t, 2H), | | and Method |
| | methyl-3-[(3-methyl-4- | 7.77 (d, 1H), 7.62 (d, 1H), | | 55 |
| | oxo-3,4-dihydro- | 7.49-7.42 (m, 3H), 7.24 (d, 1H), 3.48 (s, | | |
| | quinazolin-6-yl)amino]- | 3H), 2.18 (s, 3H), 1.78 (s, 6H) | | |
| | phenyl}nicotinamide | | | |
| 52 | N-{4-Methyl-3-[(3- | 10.24 (s, 1H), 8.48 (d, 1H), | 471 | Method 232 |
| | methyl-4-oxo-3,4- | 8.44 (d, 1H), 8.14 (s, 1H), 7.98 (s, 1H), | | and Method |
| | dihydroquinazolin-6- | 7.73 (d, 1H), 7.68 (t, 1H), 7.54 (d, | | 54 |
| | yl)amino]phenyl}-5- | 1H), 7.43-7.39 (m, 3H), 7.22 (d, | | |
| | morpholin-4- | 1H), 3.77-3.73 (m, 4H), 3.44 (s, | | |
| | ylnicotinamide | 3H), 3.25-3.22 (m, 4H), 2.17 (s, | | |
| | | 3H) | | |
| 53 | N-{4-Methyl-3-[(3- | 10.16 (s, 1H), 8.37-8.35 (m, 2H), | 469 | Method 232 |
| | methyl-4-oxo-3,4- | 8.08 (s, 1H), 7.93 (s, 1H), 7.67 (d, | | and Method |
| | dihydroquinazolin-6- | 1H), 7.59 (t, 1H), 7.47 (d, 1H), | | 52 |
| | yl)amino]phenyl}-5- | 7.37-7.34 (m, 3H), 7.16 (d, 1H), | | |
| | piperidin-1- | 3.42 (s, 3H), 3.22-3.18 (m, 4H), | | |
| | ylnicotinamide | 2.11 (s, 3H), 1.56-1.50 (m, 6H) | | |
| 54 | N-{4-Methyl-3-[(3- | 10.05 (s, 1H), 8.13 (s, 1H), 7.97 (s, | 469 | Method 232 |
| | methyl-4-oxo-3,4- | 1H), 7.75 (d, 1H), 7.53 (d, 1H), | | and Method |
| | dihydroquinazolin-6- | 7.43-7.38 (m, 4H), 7.28-7.19 (m, | | 53 |
| | yl)amino]phenyl}-3- | 3H), 7.11-7.09 (m, 1H), 3.44 (s, | | |
| | piperidin-1-ylbenzamide | 3H), 3.22-3.17 (m, 4H), 2.16 (s, | | |
| | | 3H), 1.60-1.50 (m, 6H) | | |
| 55 | 2-(1-Cyano-1- | 10.46 (s, 1H), 8.77 (d, 1H), | 453 | Method 232 |
| | methylethyl)-N-{4- | 8.14 (s, 1H), 8.00 (s, 1H), 7.95 (s, 1H), | | and Method |
| | methyl-3-[(3-methyl-4- | 7.82 (d, 1H), 7.72 (d, 1H), 7.54 (d, | | 184 |
| | oxo-3,4-dihydro- | 1H), 7.44-7.40 (m, 3H), 7.25 (d, | | |
| | quinazolin-6-yl)amino]- | 1H), 3.44 (s, 3H), 2.18 (s, 3H), | | |
| | phenyl}isonicotinamide | 1.74 (s, 6H) | | |
| 56 | 3-Cyclopropyl-N-{4- | 10.10 (s, 1H), 8.13 (s, 1H), 7.98 (s, | 426 | Method 232 |
| | methyl-3-[(3-methyl-4- | 1H), 7.75 (d, 1H), 7.65 (d, 1H), | | and Method |
| | oxo-3,4-dihydro- | 7.57-7.52 (m, 2H), 7.44-7.33 (m, | | 56 |
| | quinazolin-6-yl)amino]- | 4H), 7.27-7.19 (m, 2H), 3.44 (s, | | |
| | phenyl}benzamide | 3H), 2.16 (s, 3H), 2.01-1.95 (m, | | |
| | | 1H), 1.01-0.94 (m, 2H), | | |
| | | 0.76-0.71 (m, 2H) | | |
| 57 | N-{4-Methyl-3-[(3- | 10.24 (s, 1H), 8.14 (s, 1H), | 499 | Method 232 |
| | methyl-4-oxo-3,4- | 7.99-7.94 (m, 3H), 7.75 (d, 1H), | | and Method |
| | dihydroquinazolin-6- | 7.59-7.52 (m, 3H), 7.44-7.38 (m, 3H), | | 57 |
| | yl)amino]phenyl}-3- | 7.22 (d, 1H), 3.75-3.73 (m, 4H), | | |
| | (morpholin-4- | 3.44 (s, 3H), 3.25-3.20 (m, 4H), | | |
| | ylcarbonyl)benzamide | 2.17 (s, 3H) | | |
| 58 | N,N-Dimethyl-N′-{4- | 10.23 (s, 1H), 8.13 (s, 1H), | 457 | Method 232 |
| | methyl-3-[(3-methyl-4- | 7.99-7.93 (m, 3H), 7.75 (d, 1H), | | and Method |
| | oxo-3,4-dihydro- | 7.59-7.39 (m, 6H), 7.21 (d, 1H), 3.44 (s, | | 58 |
| | quinazolin-6-yl)amino]- | 3H), 2.99 (s, 3H), 2.90 (s, 3H), | | |
| | phenyl}isophthalamide | 2.17 (s, 3H) | | |
| 59 | 3-(1-Cyano-1- | 10.19 (s, 1H), 8.14 (s, 1H), 7.98 (s, | 457 | Method 232 |
| | methylethyl)-1-methyl- | 1H), 7.67 (d, 1H), 7.54 (d, 1H), | | and Method |
| | N-{4-methyl-3-[(3- | 7.42-7.39 (m, 3H), 7.22 (d, 1H), | | 59 |
| | methyl-4-oxo-3,4- | 7.10 (s, 1H), 4.03 (s, 3H), 3.44 (s, | | |
| | dihydroquinazolin-6- | 3H), 2.17 (s, 3H), 1.66 (s, 6H) | | |
| | yl)amino]phenyl}-1H- | | | |
| | pyrazole-5-carboxamide | | | |
| 60 | 5-(1-Cyano-1-methylethyl)- | 10.03 (s, 1H), 8.13 (s, 1H), 7.97 (s, | 457 | Method 232 |
| | 1-methyl-N-{4- | 1H), 7.78 (d, 1H), 7.53 (d, 1H), | | and Method |
| | methyl-3-[(3-methyl-4- | 7.42-7.39 (m, 3H), 7.18 (d, 1H), | | 60 |
| | oxo-3,4-dihydro- | 6.78 (s, 1H), 4.08 (s, 3H), 3.44 (s, | | |
| | quinazolin-6-yl)amino]- | 3H), 2.15 (s, 3H), 1.77 (s, 6H) | | |
| | phenyl}-1H-pyrazole-3- | | | |
| | carboxamide | | | |
| 61 | 5-(1-Cyano-1- | 10.02 (s, 1H), 8.14 (s, 1H), 7.97 (s, | 443 | Method 232 |
| | methylethyl)-N-{4- | 1H), 7.69 (d, 1H), 7.53 (d, 1H), | | and Method |
| | methyl-3-[(3-methyl-4- | 7.45-7.35 (m, 3H), 7.28 (d, 1H), | | 61 |
| | oxo-3,4-dihydro- | 7.22 (d, 1H), 6.65 (d, 1H), 3.44 (s, | | |
| | quinazolin-6-yl)amino]- | 3H), 2.17 (s, 3H), 1.72 (s, 6H) | | |
| | phenyl}-2-furamide | | | |
| 62 | 5-(1-Cyano-1- | 9.99 (s, 1H), 8.28 (d, 1H), 8.14 (s, | 459 | Method 232 |
| | methylethyl)-N-{4- | 1H), 7.97 (s, 1H), 7.70 (d, 1H), | | and Method |
| | methyl-3-[(3-methyl-4- | 7.65 (d, 1H), 7.53 (d, 1H), | | 146 |
| | oxo-3,4-dihydro- | 7.43-7.36 (m, 3H), 7.21 (d, 1H), 3.44 (s, | | |
| | quinazolin-6-yl)amino]- | 3H), 2.16 (s, 3H), 1.77 (s, 6H) | | |
| | phenyl}thiophene-3- | | | |
| | carboxamide | | | |
| 63 | 5-(1-Cyano-1- | 10.71 (s, 1H), 8.19 (d, 1H), | 444 | Method 232 |
| | methylethyl)-N-{4- | 8.02 (s, 1H), 7.74 (d, 1H), 7.54 (d, 1H), | | and Method |
| | methyl-3-[(3-methyl-4- | 7.44-7.39 (m, 3H), 7.53 (d, 1H), | | 62 |
| | oxo-3,4-dihydro- | 7.22 (d, 1H), 7.07 (s, 1H), 3.45 (s, | | |
| | quinazolin-6-yl)amino]- | 3H), 2.17 (s, 3H), 1.78 (s, 6H) | | |
| | phenyl}isoxazole-3- | | | |
| | carboxamide | | | |
| 64 | 5-Bromo-N-{4-methyl-3- | | 465 | Method 232 |
| | [(3-methyl-4-oxo-3,4- | | | and 5- |
| | dihydroquinazolin-6- | | | Bromo |
| | yl)amino]phenyl}- | | | nicotinic |
| | nicotinamide | | | acid |
| 65 | 3-(Aminosulfonyl)-N-{4- | 10.41 (s, 1H), 8.34 (m, 1H), | 464 | Method 232 |
| | methyl-3-[(3-methyl-4- | 8.13 (m, 2H), 7.98 (m, 2H), 7.72 (m, | | and Method |
| | oxo-3,4-dihydro- | 2H), 7.53 (d, 1H), 7.45 (m, 5H), | | 171 |
| | quinazolin-6-yl)amino]- | 7.22 (d, 1H), 3.44 (s, 3H), 2.17 (s, | | |
| | phenyl}benzamide | 3H) | | |
| 66 | 3-{[4-(Hydroxymethyl)- | 10.42 (s, 1H), 8.32 (m, 1H), | 562 | Method 232 |
| | piperidin-1-yl]sulfonyl}- | 8.16 (m, 2H), 7.98 (m, 2H), 7.73 (m, | | and Method |
| | N-{4-methyl-3-[(3- | 2H), 7.54 (d, 1H), 7.44 (m, 3H), | | 172 |
| | methyl-4-oxo-3,4- | 7.23 (d, 1H), 4.78 (m, 1H), | | |
| | dihydroquinazolin-6-yl)- | 3.90 (m, 1H), 3.67 (m, 1H), 3.44 (m, | | |
| | amino]phenyl}- | 4H), 2.98 (m, 1H), 1.70 (m, 1H), | | |
| | benzamide | 1.42 (m, 3H), 1.15 (m, 2H) | | |
| 67 | 3-{[3-(Hydroxymethyl)- | 10.44 (s, 1H), 8.25 (m, 1H), | 562 | Method 232 |
| | piperidin-1-yl]sulfonyl}- | 8.20 (m, 1H), 8.14 (m, 1H), 7.99 (m, | | and Method |
| | N-{4-methyl-3-[(3- | 1H), 7.89 (m, 1H), 7.79 (m, 1H), | | 173 |
| | methyl-4-oxo-3,4- | 7.75 (m, 1H), 7.54 (d, 1H), | | |
| | dihydroquinazolin-6- | 7.44 (m, 3H), 7.23 (d, 1H), 4.58 (m, | | |
| | yl)amino]phenyl}- | 1H), 3.65 (m, 1H), 3.54 (m, 1H), | | |
| | benzamide | 3.44 (s, 3H), 3.28 (m, 1H), | | |
| | | 3.14 (m, 1H), 2.24 (m, 1H), 2.18 (s, | | |
| | | 3H), 1.97 (m, 1H), 1.68 (m, 1H), | | |
| | | 1.51 (m, 1H), 0.84 (m, 1H) | | |
| 68 | 3-{[2-(Hydroxymethyl)- | 10.42 (s, 1H), 8.23 (m, 2H), | 562 | Method 232 |
| | piperidin-1-yl]sulfonyl}- | 8.14 (m, 1H), 7.98 (m, 1H), 7.89 (d, | | and Method |
| | N-{4-methyl-3-[(3- | 1H), 7.76 (m, 2H), 7.52 (d, 1H), | | 174 |
| | methyl-4-oxo-3,4- | 7.43 (m, 3H), 7.23 (d, 1H), | | |
| | dihydroquinazolin-6- | 4.46 (m, 1H), 3.66 (m, 2H), 3.44 (s, | | |
| | yl)amino]phenyl}- | 3H), 3.18 (m, 2H), 2.21 (m, 5H), | | |
| | benzamide | 1.68 (m, 2H), 1.26 (m, 1H), | | |
| | | 1.14 (m, 2H) | | |
| 69 | 3-{[Methoxy(methyl)- | 10.46 (s, 1H), 8.34 (m, 2H), | 508 | Method 232 |
| | amino]sulfonyl}-N-{4- | 8.14 (m, 1H), 7.99 (m, 2H), 7.83 (m, | | and Method |
| | methyl-3-[(3-methyl-4- | 1H), 7.74 (m, 1H), 7.54 (d, 1H), | | 175 |
| | oxo-3,4-dihydro- | 7.44 (m, 3H), 7.23 (d, 1H), 3.73 (s, | | |
| | quinazolin-6-yl)amino]- | 3H), 3.44 (s, 3H), 2.75 (s, 3H), | | |
| | phenyl}benzamide | 2.18 (s, 3H) | | |
| 70 | 3-{[(2,3-Dihydroxy- | 10.47 (s, 1H), 8.24 (m, 2H), | 552 | Method 232 |
| | propyl)(methyl)amino]- | 8.14 (m, 1H), 7.93 (m, 2H), 7.78 (m, | | and Method |
| | sulfonyl}-N-{4-methyl- | 2H), 7.53 (d, 1H), 7.44 (m, 3H), | | 176 |
| | 3-[(3-methyl-4-oxo-3,4- | 7.23 (d, 1H), 4.89 (m, 1H), | | |
| | dihydroquinazolin-6- | 4.64 (m, 1H), 3.62 (m, 1H), 3.44 (s, | | |
| | yl)amino]phenyl}- | 3H), 3.30 (m, 2H), 3.10 (m, 1H), | | |
| | benzamide | 2.85 (m, 1H), 2.76 (s, 3H), 2.18 (s, | | |
| | | 3H) | | |
| 71 | N-{4-Methyl-3-[(3- | 10.42 (s, 1H), 8.31 (m, 1H), | 548 | Method 232 |
| | methyl-4-oxo-3,4- | 8.16 (d, 1H), 8.14 (m, 1H), 7.97 (m, | | and Method |
| | dihydroquinazolin-6- | 2H), 7.86 (t, 1H), 7.73 (m, 2H), | | 177 |
| | yl)amino]phenyl}-3- | 7.54 (d, 1H), 7.43 (m, 3H), | | |
| | {[(tetrahydrofuran-2- | 7.23 (d, 1H), 3.78 (m, 1H), 3.63 (m, | | |
| | ylmethyl)amino]- | 1H), 3.53 (m, 1H), 3.44 (s, 3H), | | |
| | sulfonyl}benzamide | 2.78 (m, 2H), 2.18 (s, 3H), | | |
| | | 1.81 (m, 1H), 1.73 (m, 2H), 1.50 (m, | | |
| | | 1H) | | |
| 72 | N-{4-Methyl-3-[(3- | 10.45 (s, 1H), 8.28 (d, 1H), | 534 | Method 232 |
| | methyl-4-oxo-3,4- | 8.21 (m, 1H), 8.14 (m, 1H), 8.00 (m, | | and Method |
| | dihydroquinazolin-6- | 1H), 7.91 (d, 1H), 7.80 (t, 1H), | | 178 |
| | yl)amino]phenyl}-3- | 7.75 (m, 1H), 7.54 (d, 1H), | | |
| | (morpholin-4-ylsulfonyl)- | 7.44 (m, 3H), 7.24 (d, 1H), 3.62 (m, | | |
| | benzamide | 4H), 3.44 (s, 3H), 2.88 (m, 4H), | | |
| | | 2.18 (s, 3H) | | |
| 73 | 3-(Azetidin-1- | 10.51 (s, 1H), 8.30 (m, 2H), | 504 | Method 232 |
| | ylsulfonyl)-N-{4-methyl- | 8.15 (m, 1H), 8.02 (m, 1H), 7.98 (d, | | and Method |
| | 3-[(3-methyl-4-oxo-3,4- | 1H), 7.83 (t, 1H), 7.76 (m, 1H), | | 179 |
| | dihydroquinazolin-6- | 7.54 (d, 1H), 7.44 (m, 3H), | | |
| | yl)amino]phenyl}- | 7.24 (d, 1H), 3.69 (t, 4H), 3.44 (s, 3H), | | |
| | benzamide | 2.18 (s, 3H), 1.98 (m, 2H) | | |
| 74 | 3-[(Cyclopropylamino)- | 10.48 (s, 1H), 8.35 (m, 1H), | 504 | Method 232 |
| | sulfonyl]-N-{4-methyl-3- | 8.21 (d, 1H), 8.04 (d, 1H), 7.98 (d, 1H), | | and Method |
| | [(3-methyl-4-oxo-3,4- | 7.76 (m, 2H), 7.54 (d, 1H), | | 170 |
| | dihydroquinazolin-6- | 7.44 (m, 3H), 7.23 (d, 1H), 3.44 (s, 3H), | | |
| | yl)amino]phenyl}- | 2.18 (s, 3H), 2.10 (m, 1H), | | |
| | benzamide | 0.46 (m, 2H), 0.36 (m, 2H) | | |
| 75 | 3-[(Dimethylamino)- | 10.43 (s, 1H), 8.24 (m, 2H), | 492 | Method 232 |
| | sulfonyl]-N-{4-methyl-3- | 8.14 (m, 1H), 7.98 (m, 1H), 7.92 (d, | | and Method |
| | [(3-methyl-4-oxo-3,4- | 1H), 7.76 (m, 2H), 7.54 (d, 1H), | | 169 |
| | dihydroquinazolin-6- | 7.44 (m, 3H), 7.24 (d, 1H), 3.44 (s, | | |
| | yl)amino]phenyl}- | 3H), 2.63 (s, 6H), 2.18 (s, 3H) | | |
| | benzamide | | | |
| 76 | N-{4-Methyl-3-[(3- | 10.55 (s, 1H), 8.46 (m, 1H), | 463 | Method 232 |
| | methyl-4-oxo-3,4- | 8.27 (d, 1H), 8.14 (m, 1H), 8.10 (d, | | and 3- |
| | dihydroquinazolin-6- | 1H), 8.04 (d, 1H), 7.77 (m, 2H), | | (methyl- |
| | yl)amino]phenyl}-3- | 7.53 (d, 1H), 7.44 (m, 3H), | | sulfonyl)- |
| | (methylsulfonyl)- | 7.23 (d, 1H), 3.44 (s, 3H), 3.28 (s, 3H), | | benzoic acid |
| | benzamide | 2.18 (s, 3H) | | |
| 77 | 6-Methyl-N-{4-methyl- | 10.36 (s, 1H), 8.14 (s, 1H), 8.00 (s, | 400 | Method 232 |
| | 3-[(3-methyl-4-oxo-3,4- | 1H), 7.87-7.95 (m, 3H), 7.50 (dd, | | and 6- |
| | dihydroquinazolin-6- | 3H), 7.44 (s, 2H), 7.23 (d, 1H), | | Methylpyridine- |
| | yl)amino]phenyl}- | 3.44 (s, 3H), 2.59 (s, 3H), 2.18 (s, | | 2- |
| | pyridine-2-carboxamide | 3H) | | carboxylic |
| | | | | acid |
| 78 | 4-Methoxy-N-{4-methyl- | 10.32 (s, 1H), 8.28 (d, 1H), | 483 | Method 232 |
| | 3-[(3-methyl-4-oxo-3,4- | 8.20 (s, 1H), 8.14 (s, 1H), 8.06 (s, 1H), | | and 4- |
| | dihydroquinazolin-6- | 7.75 (s, 1H), 7.50-7.55 (m, 1H), | | Methoxy-3- |
| | yl)amino]phenyl}-3- | 7.35-7.47 (m, 4H), 7.21 (d, 1H), | | (trifluoromethyl)- |
| | (trifluoromethyl)- | 3.95 (s, 3H), 3.44 (s, 3H), 2.17 (s, | | benzoic |
| | benzamide | 3H) | | acid |
| 79 | 2-Methyl-N-{4-methyl- | 10.56 (s, 1H), 8.14 (s, 1H), | 467 | Method 232 |
| | 3-[(3-methyl-4-oxo-3,4- | 8.00 (d, 2H), 7.78-7.89 (m, 2H), | | and 2- |
| | dihydroquinazolin-6- | 7.64 (s, 1H), 7.49-7.57 (m, 1H), | | Methyl-5- |
| | yl)amino]phenyl}-5- | 7.41 (s, 1H), 7.37 (d, 1H), | | (trifluoromethyl)- |
| | (trifluoromethyl)- | 7.20-7.28 (m, 1H), 3.44 (s, 3H), 2.16 (s, 3H) | | benzoic |
| | benzamide | | | acid |
| 80 | 3-(1-Cyano-1-methylethyl)- | 10.26 (s, 1H), 8.13 (m, 1H), | 470 | Method 232 |
| | 5-fluoro-N-{4- | 7.97 (m, 1H), 7.87 (t, 1H), 7.75 (m, | | and Method |
| | methyl-3-[(3-methyl-4- | 1H), 7.72 (d, 1H), 7.60 (m, 1H), | | 208 |
| | oxo-3,4-dihydro- | 7.54 (d, 1H), 7.42 (m, 3H), | | |
| | quinazolin-6-yl)amino]- | 7.24 (d, 2H), 3.44 (s, 3H), 2.18 (s, 3H), | | |
| | phenyl}benzamide | 1.73 (s, 6H) | | |
| 81 | 3-(2-Methoxy-1,1- | 10.11 (s, 1H), 8.13 (m, 1H), | 471 | Method 232 |
| | dimethylethyl)-N-{4- | 7.97 (m, 1H), 7.87 (t, 1H), 7.74 (m, | | and Method |
| | methyl-3-[(3-methyl-4- | 2H), 7.52 (m, 2H), 7.42 (m, 4H), | | 209 |
| | oxo-3,4-dihydro- | 7.22 (d, 1H), 3.44 (s, 3H), 3.39 (s, | | |
| | quinazolin-6-yl)amino]- | 2H), 2.17 (s, 3H), 1.28 (s, 6H) | | |
| | phenyl}benzamide | | | |
| 82 | 5-(1-Cyano-1-methylethyl)- | 10.38 (s, 1H), 8.13 (m, 1H), | 470 | Method 232 |
| | 2-fluoro-N-{4- | 7.98 (m, 1H), 7.68 (m, 3H), 7.52 (d, | | and Method |
| | methyl-3-[(3-methyl-4- | 1H), 7.39 (m, 4H), 7.22 (d, 1H), | | 64 |
| | oxo-3,4-dihydro- | 3.44 (s, 3H), 2.17 (s, 3H), 1.70 (s, | | |
| | quinazolin-6-yl)amino]- | 6H) | | |
| | phenyl}benzamide | | | |
| 83 | 3-[2-(Dimethylamino)- | 10.16 (s, 1H), 8.21 (m, 1H), | 498 | Method 232 |
| | 1,1-dimethyl-2-oxo- | 8.00 (m, 1H), 7.80 (m, 1H), 7.74 (d, | | and Method |
| | ethyl]-N-{4-methyl-3- | 1H), 7.73 (t, 1H), 7.54 (d, 1H), | | 65 |
| | [(3-methyl-4-oxo-3,4- | 7.48 (t, 1H), 7.42 (m, 3H), | | |
| | dihydroquinazolin-6- | 7.32 (m, 1H), 7.22 (d, 1H), 3.45 (s, 3H), | | |
| | yl)amino]phenyl}- | 2.79 (bs, 3H), 2.42 (bs, 3H), | | |
| | benzamide | 2.17 (s, 3H), 1.46 (s, 6H) | | |
| 84 | 3-(1-Cyano-1- | 10.23 (s, 1H), 8.13 (m, 1H), | 470 | Method 232 |
| | methylethyl)-4-fluoro-N-{4- | 8.00 (m, 1H), 7.94 (m, 2H), 7.72 (m, | | and Method |
| | methyl-3-[(3-methyl-4- | 1H), 7.53 (d, 1H), 7.43 (m, 4H), | | 66 |
| | oxo-3,4-dihydro- | 7.23 (d, 1H), 3.44 (s, 3H), 2.17 (s, | | |
| | quinazolin-6-yl)amino]- | 3H), 1.77 (s, 6H) | | |
| | phenyl}benzamide | | | |
| 85 | 3-(1-Cyano-1- | 10.43 (s, 1H), 8.16 (m, 1H), | 470 | Method 232 |
| | methylethyl)-2-fluoro-N-{4- | 7.99 (m, 1H), 7.67 (d, 1H), 7.61 (m, | | and Method |
| | methyl-3-[(3-methyl-4- | 2H), 7.53 (d, 1H), 7.43 (m, 4H), | | 185 |
| | oxo-3,4-dihydro- | 7.23 (d, 1H), 3.44 (s, 3H), 2.17 (s, | | |
| | quinazolin-6-yl)amino]- | 3H), 1.76 (s, 6H) | | |
| | phenyl}benzamide | | | |
| 86 | 2-{2-Fluoro-3-[({4- | 10.34 (s, 1H), 8.18 (m, 1H), | 489 | Method 232 |
| | methyl-3-[(3-methyl-4- | 8.00 (m, 1H), 7.68 (d, 1H), 7.53 (d, | | and Method |
| | oxo-3,4-dihydro- | 1H), 7.49 (m, 2H), 7.41 (m, 1H), | | 186 |
| | quinazolin-6-yl)amino]- | 7.37 (m, 2H), 7.26 (t, 1H), | | |
| | phenyl}amino)carbonyl]- | 7.21 (d, 1H), 3.45 (s, 3H), 2.16 (s, 3H), | | |
| | phenyl}-2-methyl | 1.48 (s, 6H) | | |
| | propanoic acid | | | |
| 87 | 2-Chloro-N-{4-methyl-3- | 10.55 (s, 1H), 8.13 (m, 1H), | 487 | Method 232 |
| | [(3-methyl-4-oxo-3,4- | 7.99 (m, 1H), 7.95 (m, 1H), 7.87 (m, | | and 2- |
| | dihydroquinazolin-6- | 1H), 7.64 (m, 2H), 7.53 (d, 1H), | | Chloro-3- |
| | yl)amino]phenyl}-3- | 7.38 (m, 3H), 7.22 (d, 1H), 3.44 (s, | | (trifluoromethyl)- |
| | (trifluoromethyl)- | 3H), 2.16 (s, 3H) | | benzoic |
| | benzamide | | | acid |
| 88 | 2-Fluoro-N-{4-methyl-3- | 10.55 (s, 1H), 8.13 (m, 1H), | 471 | Method 232 |
| | [(3-methyl-4-oxo-3,4- | 7.94 (m, 3H), 7.65 (m, 1H), 7.52 (m, | | and 2- |
| | dihydroquinazolin-6- | 2H), 7.42 (m, 2H), 7.35 (m, 1H), | | Fluoro-3- |
| | yl)amino]phenyl}-3- | 7.23 (d, 1H), 3.44 (s, 3H), 2.16 (s, | | (trifluoromethyl)- |
| | (trifluoromethyl)- | 3H) | | benzoic |
| | benzamide | | | acid |
| 89 | N-{4-Methyl-3-[(3- | 10.61 (s, 1H), 9.32 (d, 1H), | 455 | Method 232 |
| | methyl-4-oxo-3,4- | 8.32 (d, 1H), 8.14 (m, 1H), 8.00 (m, | | and Method |
| | dihydroquinazolin-6-yl)- | 1H), 7.83 (d, 1H), 7.55 (d, 1H), | | 67 |
| | amino]phenyl}-2- | 7.46 (m, 3H), 7.27 (d, 1H), | | |
| | (trifluoromethyl)- | 3.45 (s, 3H), 2.20 (s, 3H) | | |
| | pyrimidine-4- | | | |
| | carboxamide | | | |
| 90 | 4-Dimethylaminomethyl- | 10.90 (s, br, 1H, HCl), 10.40 (s, | 509 | Method 232 |
| | N-[4-methyl-3-(3- | 1H), 8.46-7.20 (m, 11H), 4.45 (s, | | and Method |
| | methyl-4-oxo-3,4- | 2H), 3.40 (s, 3H), 2.70 (s, 6H), | | 68 |
| | dihydro-quinazolin-6- | 2.10 (s, 3H) | | |
| | ylamino)-phenyl]-3- | | | |
| | trifluoromethyl- | | | |
| | benzamide | | | |
| 91 | Benzo[1,3]dioxole-5- | 10.05 (s, 1H), 8.20-7.10 (m, 11H), | 428 | Method 232 |
| | carboxylic acid [4- | 6.15 (s, 2H), 3.50 (s, 3H), 2.21 (s, | | and |
| | methyl-3-(3-methyl-4- | 3H) | | Piperonylic |
| | oxo-3,4-dihydro- | | | acid |
| | quinazolin-6-ylamino)- | | | |
| | phenyl]amide | | | |
| 92 | 3-(1-Cyclopropyl-1- | 7.99 (s, 2H), 7.77-7.57 (m, 3H), | 468 | Method 232 |
| | hydroxyethyl)-N-{4- | 7.43 (d, 3H), 7.37-7.30 (m, 2H), | | and Method |
| | methyl-3-[(3-methyl-4- | 7.26 (d, 1H), 7.12 (d, 1H), 5.40 (s, | | 69 |
| | oxo-3,4-dihydro- | 1H), 3.45 (s, 3H), 2.16 (s, 3H), | | |
| | quinazolin-6-yl)amino]- | 1.82 (s, 1H), 1.40 (s, 3H), | | |
| | phenyl}benzamide | 0.89-0.74 (m, 1H), 0.41-0.25 (m, 3H) | | |
| 93 | 3-[Cyclopropyl(hydroxy)- | 10.17 (s, 1H), 8.28 (s, 1H), 8.04 (s, | 454 | Method 232 |
| | methyl]-N-{4-methyl-3- | 1H), 7.91 (s, 1H), 7.82-7.73 (m, | | and Method |
| | [(3-methyl-4-oxo-3,4- | 2H), 7.55 (d, 2H), 7.48-7.38 (m, | | 70 |
| | dihydroquinazolin-6- | 4H), 7.22 (d, 1H), 4.02 (d, 1H), | | |
| | yl)amino]phenyl}- | 3.46 (s, 3H), 2.16 (s, 3H), | | |
| | benzamide | 1.12-0.98 (m, 1H), 0.47-0.32 (m, 4H) | | |
| 94 | 3-(1,1-Dimethylpropyl)- | 10.12 (s, 1H), 8.22 (s, 1H), 8.01 (s, | 454 | Method 232 |
| | N-{4-methyl-3-[(3- | 1H), 7.83-7.79 (m, 1H), | | and Method |
| | methyl-4-oxo-3,4- | 7.78-7.70 (m, 2H), 7.56-7.50 (m, 2H), | | 220 |
| | dihydroquinazolin-6- | 7.45-7.39 (m, 4H), 7.22 (d, 1H), | | |
| | yl)amino]phenyl}- | 3.44 (s, 3H), 2.16 (s, 3H), 1.64 (q, | | |
| | benzamide | 2H), 1.27 (s, 6H), 0.61 (t, 3H) | | |
| 95 | 3-(1,1-Dimethylprop-2- | 10.18 (s, 1H), 8.25 (s, 1H), 8.04 (s, | 450 | Method 232 |
| | yn-1-yl)-N-{4-methyl-3- | 2H), 7.82-7.71 (m, 3H), | | and Method |
| | [(3-methyl-4-oxo-3,4- | 7.57-7.46 (m, 2H), 7.46-7.39 (m, 4H), | | 219 |
| | dihydroquinazolin-6- | 7.24 (d, 1H), 3.44 (s, 3H), 3.28 (s, | | |
| | yl)amino]phenyl}- | 1H), 2.17 (s, 3H), 1.55 (s, 7H) | | |
| | benzamide | | | |
| 96 | 3-(1,1-Difluoroethyl)-N- | 10.31 (s, 1H), 8.30 (s, 1H), | 448 | Method 232 |
| | {4-methyl-3-[(3-methyl- | 8.09-8.01 (m, 3H), 7.79-7.73 (m, 2H), | | and Method |
| | 4-oxo-3,4-dihydro- | 7.63 (t, 1H), 7.58-7.54 (m, 1H), | | 71 |
| | quinazolin-6-yl)amino]- | 7.47-7.41 (m, 3H), 7.24 (d, 1H), | | |
| | phenyl}benzamide | 3.46 (s, 3H), 2.17 (s, 3H), 2.01 (t, | | |
| | | 3H) | | |
| 97 | Sodium {3-(1-Cyano-1- | 10.22 (s, 1H), 8.53 (s, 1H), 7.84 (s, | 551 | Method 232 |
| | methylethyl)-5-[({4- | 2H), 7.77 (s, 1H), 7.63 (s, 1H), | | and Method |
| | methyl-3-[(3-methyl-4- | 7.57 (d, 1H), 7.48-7.39 (m, 3H), | | 72 |
| | oxo-3,4-dihydro- | 7.25 (d, 1H), 3.80 (s, 2), 3.47 (s, | | |
| | quinazolin-6-yl)amino]- | 3H), 2.17 (s, 3H), 1.72 (s, 6H) | | |
| | phenyl}amino)carbonyl]- | | | |
| | phenyl}methane | | | |
| | sulfonate | | | |
| 98 | 3-(1-Cyano-1- | 10.24 (s, 1H), 8.28 (s, 1H), 7.86 (s, | 511 | Method 232 |
| | methylethyl)-N-{4-methyl-3- | 1H), 7.81 (s, 1H), 7.74 (s, 1H), | | and Method |
| | [(3-methyl-4-oxo-3,4- | 7.65 (s, 1H), 7.58-7.52 (m, 1H), | | 73 |
| | dihydroquinazolin-6- | 7.46-7.37 (m, 3H), 7.24 (d, 1H), | | |
| | yl)amino]phenyl}-5- | 3.79 (s, 2H), 3.45 (s, 3H), 2.17 (s, | | |
| | [(methylthio)methyl]- | 3H), 1.97 (s, 3H), 1.73 (s, 6H) | | |
| | benzamide | | | |
| 99 | 3-(1-Cyano-1- | 10.23 (s, 1H), 8.14 (s, 1H), 7.99 (s, | 564 | Method 232 |
| | methylethyl)-N-{4-methyl-3- | 1H), 7.88 (s, 1H), 7.80 (s, 1H), | | and Method |
| | [(3-methyl-4-oxo-3,4- | 7.74 (s, 1H), 7.64 (s, 1H), | | 74 |
| | dihydroquinazolin-6- | 7.50-7.58 (m, 1H), 7.36-7.47 (m, 3H), | | |
| | yl)amino]phenyl}-5-[(4- | 7.24 (d, 1H), 3.59 (s, 2H), 3.45 (s, | | |
| | methylpiperazin-1- | 3H), 3.34 (s, 2H), 3.16 (s, 1H), | | |
| | yl)methyl]benzamide | 2.5-2.6 (m, 2H), 2.42-2.51 (m, | | |
| | | 4H), 2.32 (s, 3H), 2.18 (s, 3H), | | |
| | | 1.73 (s, 6H) | | |
| 100 | 3-(1-Cyano-1- | 10.65 (s, 1H), 10.41 (s, 1H), | 509 | Method 232 |
| | methylethyl)-5- | 8.46 (s, 1H), 8.09 (s, 2H), 7.97 (s, 1H), | | and Method |
| | [(dimethylamino)- | 7.77 (s, 1H), 7.60-7.45 (s, 4H), | | 75 |
| | methyl]-N-{4-methyl-3- | 7.26 (d, 1H), 4.38 (s, 2H), 3.44 (s, | | |
| | [(3-methyl-4-oxo-3,4- | 3H), 2.70-2.58 (m, 6H), 2.19 (s, | | |
| | dihydroquinazolin-6- | 3H), 1.76 (s, 6H) | | |
| | yl)amino]phenyl}- | | | |
| | benzamide | | | |
| 101 | 3-(1-Cyano-1- | 10.32 (s, 1H), 8.16 (s, 1H), 7.99 (s, | 588 | Method 232 |
| | methylethyl)-N-{4-methyl-3- | 3H), 7.75 (s, 2H), 7.51-7.60 (m, | | and Method |
| | [(3-methyl-4-oxo-3,4- | 1H), 7.40-7.50 (m, 3H), 7.24 (d, | | 77 |
| | dihydroquinazolin-6- | 1H), 3.55 (s, 2H), 3.46 (s, 3H), | | |
| | yl)amino]phenyl}-5-[3- | 3.36 (s, 3H), 2.50-2.60 (m, 4H), | | |
| | (4-methylpiperazin-1- | 2.2-2.4 (m, 4H), 2.17 (s, 3H) | | |
| | yl)prop-1-yn-1- | 2.18 (s, 3H), 1.73 (s, 6H) | | |
| | yl]benzamide | | | |
| 102 | 5-Methyl-N-{4-methyl- | 10.32 (s, 1H), 8.87 (s, 1H), 8.57 (s, | 400 | Method 232 |
| | 3-[(3-methyl-4-oxo-3,4- | 1H), 8.14 (s, 1H), 8.07 (s, 1H), | | and 5- |
| | dihydroquinazolin-6- | 8.00 (s, 1H), 7.74 (s, 1H), 7.54 (d, | | Methylpyridine- |
| | yl)amino]phenyl}- | 1H), 7.43 (s, 3H), 7.22 (d, 1H), | | 3- |
| | nicotinamide | 3.44 (s, 3H), 2.36 (s, 3H), 2.17 (s, | | carboxylic |
| | | 3H) | | acid |
| 103 | 3-tert-Butyl-N-{4- | 10.21 (s, 1H), 8.14 (s, 1H), 7.99 (s, | 441 | Method 232 |
| | methyl-3-[(3-methyl-4- | 1H), 7.87 (s, 1H), 7.83 (d, 1H), | | and Method |
| | oxo-3,4-dihydro- | 7.75 (d, 1H), 7.55-7.40 (m, 6H), | | 187 |
| | quinazolin-6-yl)amino]- | 7.18 (d, 1H), 3.43 (s, 3H), 2.15 (s, | | |
| | phenyl}benzamide | 3H), 1.29 (s, 9H) | | |
| 104 | 4-Chloro-N-{4-methyl-3- | 10.63 (s, 1H), 8.68 (d, 1H), | 419 | Method 232 |
| | [(3-methyl-4-oxo-3,4- | 8.14 (s, 1H), 8.09 (s, 1H), 7.98 (s, 1H), | | and 4- |
| | dihydroquinazolin-6- | 7.90 (s, 1H), 7.80 (d, 1H), | | Chloropyridine- |
| | yl)amino]phenyl}- | 7.49-7.56 (m, 2H), 7.41-7.48 (m, 2H), | | 2- |
| | pyridine-2-carboxamide | 7.22 (d, 1H), 3.44 (s, 3H), 2.18 (s, | | carboxylic |
| | | 3H) | | acid |
| 105 | 4-Methyl-N-{4-methyl- | 10.30 (s, 1H), 8.08-8.20 (m, 3H), | 466 | Method 232 |
| | 3-[(3-methyl-4-oxo-3,4- | 7.98 (s, 1H), 7.74 (s, 1H), | | and 4- |
| | dihydroquinazolin-6- | 7.50-7.60 (m, 2H), 7.39-7.46 (m, 3H), | | Methyl-3- |
| | yl)amino]phenyl}-3- | 7.22 (d, 1H), 3.44 (s, 3H), 3.34 (s, | | (trifluoromethyl)- |
| | (trifluoromethyl)- | 3H), 2.17 (s, 3H) | | benzoic |
| | benzamide | | | acid |
| 106 | N-{4-Methyl-3-[(3- | 11.44 (s, 1H), 10.09 (s, 1H), | 423 | Method 232 |
| | methyl-4-oxo-3,4- | 8.13 (s, 1H), 8.00 (d, 2H), 7.81 (s, 1H), | | and 1H- |
| | dihydroquinazolin-6- | 7.61 (s, 2H), 7.40-7.56 (m, 5H), | | Indole-6- |
| | yl)amino]phenyl}-1H- | 7.20 (d, 1H), 6.50 (s, 1H), 3.44 (s, | | carboxylic |
| | indole-6-carboxamide | 3H), 2.17 (s, 3H) | | acid |
| 107 | 3-Cyano-N-{4-methyl-3- | 10.33 (s, 1H), 8.36 (s, 1H), | 409 | Method 232 |
| | [(3-methyl-4-oxo-3,4- | 8.11-8.24 (m, 2H), 7.96-8.08 (m, 2H), | | and 3- |
| | dihydroquinazolin-6- | 7.65-7.79 (m, 2H), | | Cyanobenzoic |
| | yl)amino]phenyl}- | 7.49-7.59 (m, 1H), 7.38-7.47 (m, 3H), | | acid |
| | benzamide | 7.23 (d, 1H), 5.75 (s, 1H), 3.44 (s, 3H), | | |
| | | 2.18 (s, 3H) | | |
| 108 | 1,3-Dimethyl-N-{4- | 9.85 (s, 1H), 8.13 (s, 1H), 7.95 (s, | 402 | Method 232 |
| | methyl-3-[(3-methyl-4- | 1H), 7.80 (s, 1H), 7.53 (d, 1H), | | and 1,3- |
| | oxo-3,4-dihydro- | 7.38-7.46 (m, 3H), 7.16 (d, 1H), | | Dimethyl- |
| | quinazolin-6-yl)amino]- | 6.50 (s, 1H), 3.80 (s, 3H), 3.44 (s, | | 1H- |
| | phenyl}-1H-pyrazole-5- | 3H), 2.27 (s, 3H), 2.15 (s, 3H) | | pyrazole-5- |
| | carboxamide | | | carboxylic |
| | | | | acid |
| 109 | 4-Fluoro-3-methyl-N-{4- | 10.12 (s, 1H), 8.13 (s, 1H), 7.98 (s, | 416 | Method 232 |
| | methyl-3-[(3-methyl-4- | 1H), 7.86 (d, 1H), 7.73-7.82 (m, | | and 4- |
| | oxo-3,4-dihydro- | 2H), 7.48-7.58 (m, 1H), | | Fluoro-3- |
| | quinazolin-6-yl)amino]- | 7.36-7.46 (m, 3H), 7.17-7.30 (m, 2H), | | methylbenzoic |
| | phenyl}benzamide | 3.35 (s, 3H), 2.29 (s, 3H), 2.17 (s, | | acid |
| | | 3H) | | |
| 110 | 4-Methoxy-3-methyl-N- | 9.95 (s, 1H), 8.13 (s, 1H), 7.96 (s, | 428 | Method 232 |
| | {4-methyl-3-[(3-methyl- | 1H), 7.72-7.83 (m, 3H), 7.53 (d, | | and 4- |
| | 4-oxo-3,4-dihydro- | 1H), 7.38-7.46 (m, 3H), 7.19 (d, | | Methoxy-3- |
| | quinazolin-6-yl)amino]- | 1H), 7.02 (d, 1H), 3.84 (s, 3H), | | methylbenzoic |
| | phenyl}benzamide | 3.44 (s, 3H), 2.17 (d, 6H) | | acid |
| 111 | N-{4-Methyl-3-[(3- | 9.69 (s, 1H), 8.13 (s, 1H), 8.01 (s, | 426 | Method 232 |
| | methyl-4-oxo-3,4- | 1H), 7.74 (s, 1H), 7.48-7.58 (m, | | and 2,3- |
| | dihydroquinazolin-6- | 2H), 7.35-7.44 (m, 3H), | | Dihydro-1- |
| | yl)amino]phenyl}-2,3- | 7.19-7.31 (m, 2H), 6.90-6.99 (m, 1H), | | benzofuran- |
| | dihydro-1-benzofuran-7- | 4.71 (t, 2H), 3.44 (s, 3H), 3.24 (t, | | 7-carboxylic |
| | carboxamide | 2H), 2.15 (s, 3H) | | acid |
| 112 | N-{4-Methyl-3-[(3- | 11.37 (s, 1H), 10.04 (s, 1H), | 423 | Method 232 |
| | methyl-4-oxo-3,4- | 8.22 (s, 1H), 8.13 (s, 1H), 7.98 (s, 1H), | | and 1H- |
| | dihydroquinazolin-6- | 7.81 (s, 1H), 7.69 (d, 1H), | | Indole-5- |
| | yl)amino]phenyl}-1H- | 7.40-7.55 (m, 6H), 7.20 (d, 1H), 6.55 (s, | | carboxylic |
| | indole-5-carboxamide | 1H), 3.44 (s, 3H), 2.17 (s, 3H) | | acid |
| 113 | 8-Methyl-N-{4-methyl- | 9.95 (s, 1H), 8.39-8.51 (m, 2H), | 438 | Method 232 |
| | 3-[(3-methyl-4-oxo-3,4- | 8.14 (s, 1H), 7.99 (s, 1H), 7.89 (s, | | and 8- |
| | dihydroquinazolin-6- | 1H), 7.41-7.56 (m, 4H), | | Methylimidazo |
| | yl)amino]phenyl}- | 7.12-7.24 (m, 2H), 6.89 (t, 1H), 3.44 (s, | | [1,2- |
| | imidazo[1,2-α]pyridine- | 3H), 2.55 (s, 3H), 2.17 (s, 3H) | | α]pyridine- |
| | 2-carboxamide | | | 2-carboxylic |
| | | | | acid |

### Example 114

### 3-(1-Cyano-1-methylethyl)-N-[3-({2-[(2-hydroxyethyl)amino]-3-methyl-4-oxo-3,4-dihydroquinazolin-6-yl}amino)-4-methylphenyl]benzamide

3-Chloroperoxybenzoic acid (0.073 g, 0.33 mmol) was added to a stirring solution of 3-(1-cyano-1-methylethyl)-N-(4-methyl-3-{[3-methyl-2-(methylthio)-4-oxo-3,4-dihydroquinazolin-6-yl]amino}phenyl)benzamide (Example 122; 0.070 g, 0.14 mmol) in 5 ml DCM at 25° C. for 30 min. The reaction mixture was concentrated under reduced pressure. 2-Aminoethanol was added to the crude residue and the reaction mixture was stirred at 80° C. for 30 min. The crude mixture was concentrated under reduced pressure and purified by reverse phase semi preparatory HPLC. NMR (300 MHz): 10.14 (s, 1H), 7.92 (s, 1H), 7.83 (d, 1H), 7.61-7.70 (m, 2H), 7.47-7.55 (m, 1H), 7.35 (d, 2H), 7.19-7.28 (m, 1H), 7.13 (d, 1H), 7.04 (s, 1H), 6.87 (s, 1H), 3.44-3.64 (m, 4H), 3.35 (s, 3H), 2.11 (s, 3H), 1.67 (s, 6H); m/z 511.

### Example 115

The following compound was prepared by the procedure of Example 114 using the appropriate starting material and 3-(1-cyano-1-methylethyl)-N-(4-methyl-3-{[3-methyl-2-(methylthio)-4-oxo-3,4-dihydroquinazolin-6-yl]amino}phenyl)benzamide (Example 122).

| Ex. | Compound | ¹H NMR | m/z | SM |
|---|---|---|---|---|
| 115 | 3-(1-Cyano-1- | 10.14 (s, 1H), 7.92 (s, 1H), | 495 | dimethylamine |
| | methylethyl)-N-(3- | 7.83 (d, 1H), 7.62-7.68 (m, 2H), | | |
| | {[2-(dimethylamino)- | 7.47-7.54 (m, 1H), | | |
| | 3-methyl-4-oxo-3,4- | 7.26-7.35 (m, 2H), 7.21 (s, 1H), 7.14 (d, | | |
| | dihydroquinazolin-6- | 1H), 7.04 (s, 1H), 6.86 (s, 1H), | | |
| | yl]amino}-4-methyl | 3.38 (s, 3H), 2.75 (s, 6H), | | |
| | phenyl)benzamide | 2.11 (s, 3H), 1.67 (s, 6H) | | |

### Example 116

### 5-[3-(Dimethylamino)prop-1-yn-1-yl]-N-{4-methyl-3-[(3-methyl-4-oxo-3,4-dihydroquinazolin-6-yl)amino]phenyl}nicotinamide

To a 50 ml round bottom flask charged with a magnetic stir bar was added 5-bromo-N-{4-methyl-3-[(3-methyl-4-oxo-3,4-dihydroquinazolin-6-yl)amino]phenyl}nicotinamide (Example 64; 0.200 g, 0.43 mmol) and CH₃CN (2 ml). Triethylamine (0.38 ml, 2.15 mmol) was added followed by N,N-dimethylprop-2-yn-1-amine (0.14 g, 1.72 mmol). With stirring Pd(PPh₃)₄ (0.100 g, 0.086 mmol) and CuI (0.009 g, 0.043 mmol) were added and the reaction was warmed to 60° C. for 4 h. The reaction was then diluted with EtOAc (˜25 ml), filtered through a pad of SiO₂, and concentrated in vacuo. The crude product was purified on 40 g SiO₂ using EtOAc-MeOH (10:1) as eluent giving 0.138 g of the title compound as a white solid (69%); m/z 467.

### Example 117

### 5-[3-(Dimethylamino)propyl]-N-{4-methyl-3-[(3-methyl-4-oxo-3,4-dihydroquinazolin-6-yl)amino]phenyl}nicotinamide

A 50 ml round bottom flask was charged with a magnetic stir bar, 5-[3-(dimethylamino)prop-1-yn-1-yl]-N-{4-methyl-3-[(3-methyl-4-oxo-3,4-dihydroquinazolin-6-yl)amino]phenyl}nicotinamide (Example 116; 0.05 g, 0.107 mmol), MeOH (5 ml), and 10% Pd/C (0.05 g). The reaction mixture was purged with hydrogen and placed under a hydrogen atmosphere with a balloon. The mixture was allowed to stir at 25° C. for 12 h before being filtered through a bed of Celite and concentrated in vacuo. The crude product was purified on 40 g SiO₂ using EtOAc-MeOH (5:1) as eluent giving 0.045 g the title compound as an off-white solid (89%). NMR: 10.68 (s, 1H) 10.33 (s, 1H) 9.10 (d, 1H) 8.80 (d, 1H) 8.52 (s, 2H) 8.17 (s, 1H) 7.80 (d, 1H) 7.55-7.68 (m, 1H) 7.40-7.54 (m, 2H) 7.26 (d, 1H) 3.47 (s, 3H) 2.97-3.09 (m, 2H) 2.81 (t, 2H) 2.72 (d, 7H) 2.18 (s, 3H) 2.07 (d, 1H); m/z 472.

### Example 118

The following compound was prepared by the procedure of Example 117 using the appropriate starting material.

| Ex. | Compound | ¹H NMR | m/z | SM |
|---|---|---|---|---|
| 118 | 3-(1-Cyano-1-methyl | 10.08 (s, 1H), 8.23 (d, 1H), 7.78 (m, | 535 | Example |
| | ethyl)-N-(4-methyl-3-{[4- | 1H), 7.68 (d, 1H), 7.56 (m, 1H), | | 28 |
| | oxo-3-(piperidin-4-yl | 7.49 (m, 2H), 7.36 (m, 2H), 7.22 (m, 2H), | | |
| | methyl)-3,4-dihydro | 7.16 (m, 1H), 7.02 (d, 1H), 3.65 (m, | | |
| | quinazolin-6-yl]amino} | 2H), 3.05 (m, 2H), 2.52 (m, 2H), | | |
| | phenyl)benzamide | 1.94 (s, 3H), 1.53 (m, 10H), 1.30 (m, 1H) | | |

### Example 119

### 3-(1-Cyclopropylvinyl)-N-{4-methyl-3-[(3-methyl-4-oxo-3,4-dihydroquinazolin-6-yl)amino]phenyl}benzamide

Upon purification of 3-(1-cyclopropyl-1-hydroxyethyl)-N-{4-methyl-3-[(3-methyl-4-oxo-3,4-dihydroquinazolin-6-yl)amino]phenyl})benzamide (Example 92) utilizing a Gilson HPLC (0.1% TFA in CH₃CN and water), the title compound was formed by the elimination of the hydroxyl group from the TFA present in the purification solvents. NMR: 7.99 (s, 2H), 7.71-7.57 (m, 3H), 7.43 (d, 3H), 7.37-7.30 (m, 2H), 7.26 (d, 1H), 7.12 (d, 1H), 5.40 (s, 1H), 3.45 (s, 3H), 2.16 (s, 3H), 1.82 (s, 1H), 1.40 (s, 3H), 0.89-0.74 (m, 1H), 0.41-0.25 (m, 3H); m/z 450.

### Example 120

### 4-[(Cyclopropylamino)methyl]-N-{4-methyl-3-[(3-methyl-4-oxo-3,4-dihydroquinazolin-6-yl)amino]phenyl}-3-(trifluoromethyl)benzamide

A solution of tert-butyl cyclopropyl[4-[({4-methyl-3-[(3-methyl-4-oxo-3,4-dihydroquinazolin-6-yl)amino]phenyl}amino)carbonyl]-2-(trifluoromethyl)benzyl]carbamate (Example 47; 0.088 g, 0.14 mmol) in 4 N HCl in 1,4-dioxane was stirred at 25° C. for 45 min. The reaction mixture was concentrated under reduced pressure to give the desired product. NMR: 10.47 (s, 1H), 8.53 (s, 1H), 8.22-8.32 (m, 2H), 8.09 (d, 1H) 7.73 (d, 1H) 7.57 (d, 1H) 7.39-7.45 (m, 2H) 7.36 (d, 1H) 7.20 (d, 1H) 4.36 (s, 2H) 3.43 (s, 3H) 2.69 (m, 1H) 2.12 (s, 3H) 0.87-0.97 (m, 2H) 0.64-0.74 (m, 2H); m/z 522.

### Example 121

The following compound was prepared by the procedure of Example 120 using the appropriate starting material.

| Ex. | Compound | ¹H NMR | m/z | SM |
|---|---|---|---|---|
| 121 | 3-[6-[(5-{[3-(1- | 10.32 (s, 1H), 8.54 (m, 1H), | 532 | Example 26 |
| | Cyano-1-methyl | 7.96 m, 3H), 7.75 (m, 2H), 7.60 (m, | | |
| | ethyl)benzoyl]amino}- | 2H), 7.43 (m, 2H), 7.25 (d, 1H), | | |
| | 2-methylphenyl) | 3.98 (m, 2H), 2.80 (m, 2H), | | |
| | amino]-4-oxo | 2.17 (s, 3H), 1.99 (m, 2H), 1.73 (s, | | |
| | quinazolin-3(4H)- | 6H) | | |
| | yl]propan-1-aminium | | | |
| | chloride | | | |

### Example 122

### 3-(1-Cyano-1-methylethyl)-N-(4-methyl-3-{[3-methyl-2-(methylthio)-4-oxo-3,4-dihydroquinazolin-6-yl]amino}phenyl)benzamide

A mixture of N-(3-amino-4-methylphenyl)-3-(1-cyano-1-methylethyl)benzamide (Method 87; 0.26 g, 0.88 mmol), 6-bromo-3-methyl-2-(methylthio)quinazolin-4(3H)-one (Method 182; 0.25 g, 0.88 mmol), caesium carbonate (0.857 g, 2.63 mmol), BINAP (0.040 g, 0.088 mmol) and Pd₂(dba)₃ (0.055 g, 0.044 mmol) in 1,4-dioxane (6 ml) was stirred at 100° C. for 15 h. The reaction mixture was filtered over Celite, concentrated and purified on silica gel. m/z 498.

### Preparation of Starting Materials

### Method 1

### 3-Cyanomethyl-benzoic acid methyl ester

A suspension of methyl-3-(bromomethyl)benzoate (13.5 g, 58.9 mmol) and sodium cyanide (4.33 g, 88.4 mmol) in DMF (25 ml) and water (1 ml) was stirred at 75° C. for 5 h. The reaction mixture was quenched with water (50 ml) and extracted with EtOAc (3×100 ml). The combined organics were dried and concentrated under reduced pressure. The resulting residue was purified by column chromatography utilizing an ISCO system (hexane-EtOAc) to give 7.2 g (70%) of colourless oil. NMR: 7.90 (s, 1H), 7.86 (d, 1H), 7.60 (d, 1H), 7.50 (m, 1H), 4.10 (s, 2H), 3.80 (s, 3H); m/z 175.

### Methods 2-18

The following compounds were prepared by the procedure of Method 1, using the appropriate SM and sodium cyanide.

| Meth | Compound | m/z | SM |
|---|---|---|---|
| 2 | Methyl 3-(benzyloxy)-5-(cyanomethyl)benzoate | 283 | Method 136 |
| 3 | Methyl 3-(cyanomethyl)-5-methoxybenzoate | 206 | Method 137 |
| 4 | Methyl 4-(cyanomethyl)thiophene-2-carboxylate | 182 | Method 152 |
| 5 | Ethyl 2-(cyanomethyl)-1,3-thiazole-4-carboxylate | 197 | Method 157 |
| 6 | Methyl 4-chloro-3-(cyanomethyl)benzoate | 210 | Method 156 |
| 7 | Methyl 5-(cyanomethyl)nicotinate | 177 | Method 159 |
| 8 | Methyl 3-(cyanomethyl)-1-methyl-1H-pyrazole-5- | 180 | Method 160 |
| | carboxylate | | |
| 9 | Methyl 5-(cyanomethyl)-1-methyl-1H-pyrazole-3- | 180 | Method 161 |
| | carboxylate | | |
| 10 | Methyl 5-(cyanomethyl)-2-furoate | 166 | Method 162 |
| 11 | Methyl 5-(cyanomethyl)isoxazole-3-carboxylate | 167 | Method 163 |
| 12 | [4-({[tert-Butyl(diphenyl)silyl]oxy}methyl)-2- | 393 | Method 153 |
| | thienyl]acetonitrile | | |
| 13 | (3-Bromo-5-fluorophenyl)acetonitrile | 215 | Method 138 |
| 14 | Methyl 5-(cyanomethyl)-2-fluorobenzoate | 195 | Method 164 |
| 15 | Methyl 3-(cyanomethyl)-4-fluorobenzoate | 194 | Method 168 |
| 16 | (2-Fluoro-3-methylphenyl)acetonitrile | 150 | 1-(Bromo methyl)-2- |
| | | | fluoro-3-methylbenzene |
| 17 | Methyl 3-(cyanomethyl)-5-methylbenzoate | 190 | Method 166 |
| 18 | Methyl 3-bromo-5-(cyanomethyl)benzoate | 255 | Method 139 |

### Method 19

### 3-(1-Cyano-1-methylethyl)benzoic acid methyl ester

A solution of 3-cyanomethyl-benzoic acid methyl ester (Method 1; 7.2 g, 41.1 mmol) in DMSO (80 ml) was treated with sodium hydride (60%, 4.9 g, 123.3 mmol, 3 eq). Methyl iodide was then added dropwise at 0° C. The reaction mixture was stirred at 25° C. for 12 h. The reaction mixture was then quenched with water (200 ml) and extracted with EtOAc. The combined organics were dried and concentrated under reduced pressure. The crude product was purified by column chromatography utilizing an ISCO system (hexane-EtOAc) to give 5.5 g (66%) of a colourless oil. NMR: 8.05 (s, 1H), 7.90 (d, 1H), 7.75 (d, 1H), 7.55 (m, 1H), 3.80 (s, 3H), 1.62 (s, 6H); m/z 203.

### Methods 20-39

The following compounds were prepared by the procedure of Method 19, using the appropriate SM and methyl iodide.

| Meth | Compound | m/z | SM |
|---|---|---|---|
| 20 | 3-Benzyloxy-5-(cyano-dimethyl-methyl)-benzoic acid methyl | 310 | Method 2 |
| | ester | | |
| 21 | 3-(Cyano-dimethyl-methyl)-5-methoxy-benzoic acid methyl | 234 | Method 3 |
| | ester | | |
| 22 | 2-Methyl-2-(2-thienyl)propanenitrile | 152 | 2-Thienyl |
| | | | acetonitrile |
| 23 | Methyl 4-(1-cyano-1-methylethyl)thiophene-2-carboxylate | 210 | Method 4 |
| 24 | Methyl 4-chloro-3-(1-cyano-1-methylethyl)benzoate | 238 | Method 6 |
| 25 | Ethyl 2-(1-cyano-1-methylethyl)-1,3-thiazole-4-carboxylate | 225 | Method 5 |
| 26 | Methyl 5-(1-cyano-1-methylethyl)nicotinate | 205 | Method 7 |
| 27 | Methyl 3-(1-cyano-1-methylethyl)-1-methyl-1H-pyrazole-5- | 208 | Method 8 |
| | carboxylate | | |
| 28 | Methyl 5-(1-cyano-1-methylethyl)-1-methyl-1H-pyrazole-3- | 208 | Method 9 |
| | carboxylate | | |
| 29 | Methyl 5-(1-cyano-1-methylethyl)-2-furoate | 194 | Method 10 |
| 30 | Methyl 5-(1-cyano-1-methylethyl)isoxazole-3-carboxylate | 195 | Method 11 |
| 31 | 2-[4-({[tert-Butyl(diphenyl)silyl]oxy}methyl)-2-thienyl]-2- | 421 | Method 12 |
| | methylpropanenitrile | | |
| 32 | 2-(3-Bromo-5-fluorophenyl)-2-methylpropanenitrile | 243 | Method 13 |
| 33 | Methyl 2-(3-bromophenyl)-2-methylpropanoate | 258 | Method 149 |
| 34 | 2-(3-Bromophenyl)-2-methylpropyl methyl ether | 244 | Method 134 |
| 35 | Methyl 5-(1-cyano-1-methylethyl)-2-fluorobenzoate | 222 | Method 14 |
| 36 | Methyl 3-(1-cyano-1-methylethyl)-4-fluorobenzoate | 222 | Method 15 |
| 37 | 2-(2-Fluoro-3-methylphenyl)-2-methylpropanenitrile | 178 | Method 16 |
| 38 | Methyl 3-(1-cyano-1-methylethyl)-5-methylbenzoate | 218 | Method 17 |
| 39 | Methyl 3-bromo-5-(1-cyano-1-methylethyl)benzoate | 283 | Method 18 |

### Method 40

### 3-(1-Cyano-1-methylethyl)benzoic acid

A solution of 3-(1-cyano-1-methylethyl)benzoic acid methyl ester (Method 19; 5.5 g, 27.1 mmol) in 100 ml of THF-MeOH—H₂O (3:1:1) was treated with lithium hydroxide (1.95 g) in 20 ml water. The mixture was stirred at 25° C. for 12 h. The solvents were removed under reduced pressure and the residue was diluted with water and acidified with 10% HCl to pH=1-3. The resulting white solid (4.83 g, 94%) was filtered, washed with water, and dried. NMR: 13.00 (s, 1H), 7.95 (s, 1H), 7.80 (d, 1H), 7.65 (d, 1H), 7.45 (m, 1H), 1.60 (s, 6H); m/z 189.

### Methods 41-77

The following compounds were prepared by the procedure of Method 40, using the appropriate SM and lithium hydroxide.

| Meth | Compound | m/z | SM |
|---|---|---|---|
| 41 | 3-(Benzyloxy)-5-(methoxycarbonyl)benzoic | 287 | Method 130 |
| | acid | | |
| 42 | 3-Methoxy-5-(methoxycarbonyl)benzoic acid | 211 | 5-Methoxy-isophthalic acid |
| | | | dimethyl ester |
| 43 | 3-(Cyano-dimethyl-methyl)-5-methoxy-benzoic | 220 | Method 21 |
| | acid | | |
| 44 | 3-(Cyano-dimethyl-methyl)-5-(2- | 277 | Method 141 |
| | dimethylamino-ethoxy)-benzoic acid | | |
| 45 | 4-(1-Cyano-1-methylethyl)thiophene-2- | 196 | Method 23 |
| | carboxylic acid | | |
| 46 | 4-Chloro-3-(1-cyano-1-methylethyl)benzoic | 224 | Method 24 |
| | acid | | |
| 47 | 2-(1-Cyano-1-methylethyl)-1,3-thiazole-4- | 197 | Method 25 |
| | carboxylic acid | | |
| 48 | Methyl 4-{[(tert- | 260 | Method 180 |
| | butoxycarbonyl)(cyclopropyl)amino]methyl}-3- | | |
| | (trifluoromethyl)benzoate | | |
| 49 | 3-(3,3-Dimethylbut-1-yn-1-yl)benzoic acid | 203 | Method 188 |
| 50 | 3-(3-Hydroxy-3-methylbut-1-yn-1-yl)benzoic | 205 | Method 189 |
| | acid | | |
| 51 | 3-(Cyclopropylethynyl)benzoic acid | 187 | Method 190 |
| 52 | 5-Piperidin-1-ylnicotinic acid | 207 | Method 192 |
| 53 | 3-Piperidin-1-ylbenzoic acid | 206 | Method 193 |
| 54 | 5-Morpholin-4-ylnicotinic acid | 209 | Method 194 |
| 55 | 5-(1-Cyano-1-methylethyl)nicotinic acid | 191 | Method 26 |
| 56 | 3-Cyclopropylbenzoic acid | 163 | Method 235 |
| 57 | 3-(Morpholin-4-ylcarbonyl)benzoic acid | 236 | Method 85 |
| 58 | 3-[(Dimethylamino)carbonyl]benzoic acid | 194 | Method 86 |
| 59 | 3-(1-Cyano-1-methylethyl)-1-methyl-1H- | 194 | Method 27 |
| | pyrazole-5-carboxylic acid | | |
| 60 | 5-(1-Cyano-1-methylethyl)-1-methyl-1H- | 194 | Method 28 |
| | pyrazole-3-carboxylic acid | | |
| 61 | 5-(1-Cyano-1-methylethyl)-2-furoic acid | 180 | Method 29 |
| 62 | 5-(1-Cyano-1-methylethyl)isoxazole-3- | 181 | Method 30 |
| | carboxylic acid | | |
| 63 | 2-(3-Bromophenyl)-2-methylpropanoic acid | 244 | Method 33 |
| 64 | 5-(1-Cyano-1-methylethyl)-2-fluorobenzoic | 208 | Method 35 |
| | acid | | |
| 65 | 3-[2-(Dimethylamino)-1,1-dimethyl-2- | 236 | Method 233 |
| | oxoethyl]benzoic acid | | |
| 66 | 3-(1-Cyano-1-methylethyl)-4-fluorobenzoic | 208 | Method 36 |
| | acid | | |
| 67 | 2-(Trifluoromethyl)pyrimidine-4-carboxylic | 193 | Methyl 2- |
| | acid | | (trifluoromethyl)pyrimidine- |
| | | | 4-carboxylate |
| 68 | 4-Dimethylaminomethyl-3-trifluoromethyl- | 247 | Method 214 |
| | benzoic acid | | |
| 69 | 3-(1-Cyclopropyl-1-hydroxyethyl)benzoic acid | 207 | Method 222 |
| 70 | 3-[Cyclopropyl(hydroxy)methyl]benzoic acid | 192 | Method 223 |
| 71 | 3-(1,1-Difluoroethyl)benzoic acid | 186 | Method 224 |
| 72 | Sodium [3-carboxy-5-(1-cyano-1- | 283 | Method 225 |
| | methylethyl)phenyl]methanesulfonate | | |
| 73 | 3-(1-Cyano-1-methylethyl)-5- | 249 | Method 226 |
| | [(methylthio)methyl]benzoic acid | | |
| 74 | 3-(1-Cyano-1-methylethyl)-5-[(4- | 301 | Method 215 |
| | methylpiperazin-1-yl)methyl]benzoic acid | | |
| 75 | 3-(1-Cyano-1-methylethyl)-5- | 246 | Method 216 |
| | [(dimethylamino)methyl]benzoic acid | | |
| 76 | 3-Bromo-5-(methoxycarbonyl)benzoic acid | 259 | Dimethyl 5- |
| | | | bromoisophthalate |
| 77 | 3-(1-Cyano-1-methylethyl)-5-[3-(4- | 325 | Method 227 |
| | methylpiperazin-1-yl)prop-1-yn-1-yl]benzoic | | |
| | acid | | |

### Method 78

### 3-(1-Cyano-1-methylethyl)-N-(4-methyl-3-nitro-phenyl)benzamide

A mixture of 4-methyl-3-nitroaniline (2.74 g, 18 mmol), 3-(1-cyano-1-methylethyl)benzoic acid (Method 40; 3.4 g, 18 mmol), EDCI (6.9 g, 36 mmol), HOBt (2.43 g, 18 mmol) and diisopropyl ethyl amine (3.48 g, 27 mmol) in DMF (30 ml) was stirred at 25° C. for 12 h. The reaction mixture was diluted with DCM and then washed with water. The organic phase was dried with NaCl(sat) and then Na₂SO₄(s). The solvent was removed by reduced pressure and the resulting product was purified by column chromatography utilizing an ISCO system (hexane-EtOAc) to give 4.4 g (53%). NMR: 10.50 (s, 1H), 8.40 (s, 1H), 7.40-7.95 (m, 6H), 3.20 (s, 3H), 1.65 (s, 6H); m/z 323.

### Methods 79-86

The following compounds were prepared by the procedure of Method 78, using the appropriate SM.

| Meth | Compound | m/z | SM |
|---|---|---|---|
| 79 | 3-(Cyano-dimethyl-methyl)-5-(2-dimethylamino- | 411 | Method 44 |
| | ethoxy)-N-(4-methyl-3-nitro-phenyl)-benzamide | | |
| 80 | 3-(Cyano-dimethyl-methyl)-5-methoxy-N-(4- | 354 | Method 43 |
| | methyl-3-nitro-phenyl)-benzamide | | |
| 81 | N-(4-Methyl-3-nitrophenyl)-5-(1-cyano-1- | 330 | Method 144 |
| | methylethyl)thiophene-2-carboxamide | | |
| 82 | N-(4-Methyl-3-nitrophenyl)-4-(1-cyano-1- | 330 | Method 45 |
| | methylethyl)thiophene-2-carboxamide | | |
| 83 | N-(4-Methyl-3-nitrophenyl)-6-(1-cyano-1- | 325 | Method 155 |
| | methylethyl)pyridine-2-carboxamide | | |
| 84 | N-(4-Methyl-3-nitrophenyl)-4-chloro-3-(1- | 358 | Method 46 |
| | cyano-1-methylethyl)benzamide | | |
| 85 | Methyl 3-(morpholin-4-ylcarbonyl)benzoate | 251 | Morpholine and 3- |
| | | | (methoxycarbonyl)- |
| | | | benzoic acid |
| 86 | Methyl 3-[(dimethylamino)carbonyl]benzoate | 208 | Dimethylamine and 3- |
| | | | (methoxycarbonyl)- |
| | | | benzoic acid |

### Method 87

### N-(3-Amino-4-methylphenyl)-3-(1-cyano-1-methylethyl)benzamide

A suspension of 3-(1-cyano-1-methylethyl)-N-(4-methyl-3-nitro-phenyl)benzamide (Method 78; 4 g, 13.9 mmol) and 5% Pd on carbon in hydrazine hydrate (100 ml) and ethanol (100 ml) was heated to reflux for 3 h, then stirred at 80° C. for 12 h. The Pd/C was removed by filtration and the filtrate was concentrated under reduced pressure. The residue was purified by column chromatography using an ISCO system (hexane-EtOAc) to give 3.7 g (91%) of an orange gum. NMR: 9.95 (s, 1H), 8.00 (s, 1H), 7.90 (d, 1H), 7.70 (d, 1H), 7.55 (m, 1H), 7.05 (s, 1H), 6.80-6.87 (m, 2H), 4.85 (s, 2H), 2.05 (s, 3H), 1.85 (s, 6H); m/z 293.

### Methods 88-103

The following compounds were prepared by the procedure of Method 87 using the appropriate SM.

| Meth | Compound | m/z | SM |
|---|---|---|---|
| 88 | N-(3-Amino-4-methylphenyl)-3-(trifluoromethyl) benzamide | 294 | Method 111 |
| 89 | N-(3-Amino-4-methylphenyl)-4-chloro-3-(trifluoromethyl) | 330 | Method |
| | benzamide | | 112 |
| 90 | N-(3-Amino-4-methyl-phenyl)-3-(cyano-dimethyl-methyl)-5- | 381 | Method 79 |
| | (2-dimethylamino-ethoxy)-benzamide | | |
| 91 | N-(3-Amino-4-methyl-phenyl)-3-(cyano-dimethyl-methyl)-5- | 324 | Method 80 |
| | methoxy-benzamide | | |
| 92 | N-(3-Amino-4-methylphenyl)-5-(1-cyano-1-methylethyl) | 300 | Method 81 |
| | thiophene-2-carboxamide | | |
| 93 | N-(3-Amino-4-methylphenyl)-4-(1-cyano-1-methylethyl) | 300 | Method 82 |
| | thiophene-2-carboxamide | | |
| 94 | N-(3-Amino-4-methylphenyl)-6-(1-cyano-1- | 295 | Method 83 |
| | methylethyl)pyridine-2-carboxamide | | |
| 95 | N-(3-Amino-4-methylphenyl)-4-chloro-3-(1-cyano-1- | 329 | Method 84 |
| | methylethyl)benzamide | | |
| 96 | N-(3-Amino-4-methyl-phenyl)-3-(cyano-dimethyl-methyl)-5- | 380 | Method |
| | methylcarbamoylmethoxy-benzamide | | 199 |
| 97 | N-(3-Amino-4-methylphenyl)-3-(1-cyano-1-methylethyl)-5-(2- | 423 | Method |
| | morpholin-4-ylethoxy)benzamide | | 200 |
| 98 | N-(3-Amino-4-methylphenyl)-3-(1-cyano-1-methylethyl)-5-(2- | 422 | Method |
| | piperidin-1-ylethoxy)benzamide | | 201 |
| 99 | N-(3-Amino-4-methylphenyl)-3-(1-cyano-1-methylethyl)-5-[3- | 450 | Method |
| | (4-methylpiperazin-1-yl)propoxy]benzamide | | 202 |
| 100 | N-(3-Amino-4-methylphenyl)-3-(1-cyano-1-methylethyl)-5-[2- | 421 | Method |
| | (1-methylpyrrolidin-2-yl)ethoxy]benzamide | | 203 |
| 101 | N-(3-Amino-4-methylphenyl)-3-(2-azepan-1-ylethoxy)-5-(1- | 435 | Method |
| | cyano-1-methylethyl)benzamide | | 204 |
| 102 | N-(3-Amino-4-methylphenyl)-3-(1-cyano-1-methylethyl)-5-(2- | 368 | Method |
| | methoxyethoxy)benzamide | | 205 |
| 103 | N-(3-Amino-4-methylphenyl)-3-(1-cyano-1-methylethyl)-5- | 407 | Method |
| | [(1-methylpiperidin-4-yl)oxy]benzamide | | 213 |

### Method 104

### 6-Bromo-3-methylquinazolin-4(3H)-one

2-Amino-5-bromobenzoic acid (5.00 g, 0.023 mol) was reacted with N-methylformamide (40 ml) at 180° C. for 12 h. The reaction was quenched with H₂O and the resulting precipitate was collected by vacuum filtration to give 5.26 g (95%) of a yellow-white solid; m/z 240.

### Methods 105-110

The following compounds were prepared by the procedure of Method 104, using the appropriate amino-benzoic acid (commercially available unless otherwise indicated) and the appropriate formamide as starting materials.

| Meth | Compound | m/z | SM |
|---|---|---|---|
| 105 | 7-Chloro-3-methylquinazolin-4(3H)- | 195 | Methyl 2-Amino-4-chlorobenzoate |
| | one | | |
| 106 | 6-Bromoquinazolin-4(3H)-one | 226 | 2-Amino-5-bromobenzoic acid |
| 107 | 6-Bromo-2-methylquinazolin-4(3H)- | 240 | 2-Amino-5-bromobenzoic acid |
| | one | | |
| 108 | 6-Chloro-3-methylpyrido[3,4- | 195 | Method 127 |
| | d]pyrimidin-4(3H)-one | | |
| 109 | 6-(5-Amino-2-methylphenoxy)-3- | 283 | Method 129 |
| | methylquinazolin-4(3H)-one | | |
| 110 | 6-Bromo-3-cyclopropylquinazolin- | 266 | 2-Amino-5-bromobenzoic acid and |
| | 4(3H)-one | | Method 228 |

### Method 111

### N-(4-Methyl-3-nitrophenyl)-3-trifluoromethylbenzamide

A solution of 4-methyl-3-nitro-phenylamine (3.64 g, 24 mmol) and 3-trifluoromethyl benzoyl chloride (5 g, 24 mmol) in DCM (100 ml) was treated with triethylamine (4.85 g, 48 mmol). The mixture was stirred at 25° C. for 20 min. The reaction was then quenched with water (50 ml) and stirred for 15 min. The solid was collected by vacuum filtration and washed with hexane. A second crop of solid was collected from the filtrate to give a total yield of 7.78 g (100%) of white-light yellow solid. NMR: 7.35 (m, 1H), 7.66 (m, 1H), 7.87 (m, 2H), 8.15 (m, 2H), 8.40 (s, 1H), 10.62 (s, 1H); m/z 324.

### Methods 112-113

The following compounds were prepared by the procedure of Method 111, using the appropriate benzyl chloride and amine.

| Meth | Compound | m/z | SM |
|---|---|---|---|
| 112 | 4-Chloro-N-(4-methyl-3-nitrophenyl)- | 362 | 4-Chloro-3-(trifluoromethyl)benzoyl |
| | 3-(trifluoromethyl)benzamide | | chloride (Method 114) and 4-methyl-3- |
| | | | nitro-phenylamine |
| 113 | 2-(3-Bromophenyl)-N,N,2- | 271 | Method 115 and dimethylamine |
| | trimethylpropanamide | | |

### Method 114

### 4-Chloro-3-(trifluoromethyl)benzoyl chloride

A solution of 4-chloro-3-(trifluoromethyl)benzoic acid (1.02 g, 4.54 mmol), oxalyl chloride (0.59 ml, 6.81 mmol, 1.5 equiv) and catalytic DMF (50 ml) in DCM (10 ml) was stirred at 25° C. for 12 h. The solvents were removed under reduced pressure. The resulting product was utilized without further purification; m/z 244.

### Method 115

The following compound was prepared by the procedure of Method 114 using the appropriate starting materials.

| Meth | Compound | m/z | SM |
|---|---|---|---|
| 115 | 2-(3-Bromophenyl)-2-methylpropanoyl chloride | 263 | Method 63 |

### Method 116

### 6-Bromo-3-(3-morpholin-4-ylpropyl)quinazolin-4(3H)-one

6-Bromoquinazolin-4(3H)-one (Method 106; 200 mg, 0.889 mol) and K₂CO₃ (369 mg, 2.67 mmol, 3.0 equiv) was reacted with 4-(3-chloropropyl)morpholine (145 mg, 0.889 mmol) in DMF (3 ml) at 50° C. for 12 h. The reaction was quenched with H₂O and extracted with EtOAc. The organics were dried by NaCl(sat) then Na₂SO₄(s) The solvents were removed under reduced pressure. The resulting solid (306 mg, 96%) was used without further purification; m/z 353.

### Methods 117-123

The following compounds were prepared by the procedure of Method 116, using 6-bromoquinazolin-4(3H)-one (Method 106) and the appropriate alkyl halide as starting materials.

| Meth | Compound | m/z | SM |
|---|---|---|---|
| 117 | 6-Bromo-3-ethylquinazolin-4(3H)-one | 254 | Ethyl iodide |
| 118 | 6-Bromo-3-(cyclopropylmethyl)-quinazolin-4(3H)- | 280 | Cyclopropylmethyl |
| | one | | bromide |
| 119 | 6-Bromo-3-(2,3-dihydroxypropyl)-quinazolin- | 300 | 3-Bromo-1,2- |
| | 4(3H)-one | | propanediol |
| 120 | 6-Bromo-3-(2-{[tert-butyl(dimethyl)silyl]oxy}ethyl) | 384 | (2-Bromoethoxy)-tert- |
| | quinazolin-4(3H)-one | | butyl-dimethylsilane |
| 121 | 6-Bromo-3-[(1-methylpiperidin-3- | 337 | 3-(Chloromethyl)-1- |
| | yl)methyl]quinazolin-4(3H)-one | | methylpiperidine |
| 122 | Benzyl 4-[(6-bromo-4-oxoquinazolin-3(4H)- | 457 | Method 206 |
| | yl)methyl]piperidine-1-carboxylate | | |
| 123 | tert-Butyl [3-(6-bromo-4-oxoquinazolin-3(4H)- | 383 | Method 207 |
| | yl)propyl]carbamate | | |

### Method 124

### 6-Bromo-3-[(2,2-dimethyl-1,3-dioxolan-4-yl)methyl]quinazolin-4(3H)-one

A solution of 6-bromo-3-(2,3-dihydroxypropyl)-quinazolin-4(3H)-one (Method 119; 300 mg, 1.00 mmol) in 2,2-dimethoxypropane (5 ml) was treated with p-toluenesulfonic acid (50 mg). The reaction stirred for 15 min and was then quenched with 10% NaOH(aq). The reaction mixture was extracted with EtOAc, and the organics were dried by NaCl(sat) then Na₂SO₄(s). The organics were concentrated under reduced pressure. The resulting solid was purified by column chromatography using an ISCO system (hexane-EtOAc, 1:1) to give 276 mg (81%) of an off-white solid; m/z 340.

### Method 125

### (6-Chloro-pyridin-3-yl)carbamic acid tert-butyl ester

A solution of 2-chloro-5-amino-pyridine (8.7 g, 67.7 mmol) in dioxane (85 ml) was treated with tert-butyl carbonic anhydride (16.2 g, 74.4 mmol). The resulting pale solution was heated to 80° C. for 10 h. The solvents were removed under reduced pressure to yield the desired product as, white solid; m/z 229.

### Method 126

### 5-tert-Butoxycarbonyl amino-2-chloro-isonicotinic acid

A solution of (6-chloro-pyridin-3-yl)carbamic acid tert-butyl ester (Method 125; 4.0 g, 17.5 mmol) in ether (40 ml) at −78° C. was treated with N,N,N,N-tetramethyl ethylene diamine (0.78 ml, 5.25 mmol) via a syringe followed by drop-wise addition of a solution of n-BuLi (1.6M, 32.8 ml, 52.5 mmol). The resulting deep coloured solution was kept at −78° C. for 1 hour. The reaction mixture was then warmed to 0° C. for 10 min and then cooled to −78° C. CO₂(g) was bubbled through the solution for 20 min and the resulting mixture was stirred for 10 min at 25° C. The solvent was removed under reduced pressure. The resulting residue was treated with 1N HCl solution (60 ml) resulting in a solid precipitate that was collected by filtration; m/z 273.

### Method 127

### 5-Amino-2-chloro-isonicotinic acid

A solution of 5-tert-butoxycarbonyl amino-2-chloro-isonicotinic acid (Method 126; 2.06 g, 7.6 mmol) in methanol (10 ml) at 0° C. was treated with a solution of HCl in dioxane (4N, 2.3 ml). The resulting cloudy solution stirred at 25° C. for 1 hour. The solvent was evaporated under reduced pressure to afford the desired product; m/z 173.

### Method 128

### 5-(2-Methyl-5-nitrophenoxy)-2-nitrobenzoic acid

5-Fluoro-2-nitrobenzoic acid (827 mg, 5.40 mmol), 2-methyl-5-nitrophenol (1.00 g, 5.40 mmol) and K₂CO₃ (2.21 g, 16.02 mmol, 3.0 equiv) were dissolved in DMF (10 ml). The reaction was heated to 100° C. for 48 h. The reaction was quenched with 10% HCl(aq) and extracted with EtOAc. The organics were dried with NaCl(sat) then Na₂SO₄(s). The solvents were then removed under reduced pressure to give the desired material: 1.72 g, 99%; m/z 319.

### Method 129

### 2-Amino-5-(5-amino-2-methylphenoxy)benzoic acid

5-(2-Methyl-5-nitrophenoxy)-2-nitrobenzoic acid (Method 128; 1.72 g, 5.40 mmol) was dissolved in MeOH (10 ml). Pd on carbon (30%) (100 mg) was then added. The reaction was then placed on a Parr hydrogenator at 50 psi for 5 h. The reaction mixture was then filtered through celite and the solvents were removed under reduced pressure to give a brown solid (1.30 g, 93%); m/z 259.

### Method 130

### 5-Benzyloxy-isophthalic acid dimethyl ester

A solution of dimethyl 5-hydroxyisophthalate (10.5 g, 50 mmol) in 50 ml of DMF was treated with benzyl bromide (7.3 ml, 60 mmol) dropwise. The reaction stirred for 12 h at 25° C. under nitrogen atmosphere. The reaction mixture was quenched with crushed ice and the resulting solid was collected by vacuum filtration. The solid was washed with water and air dried to provide the desired product (14 g, 95%). NMR: δ 8.2 (s, 1H), 7.9 (s, 1H), 7.2-7.6 m, 5H), 7.2 (s, 1H), 5.2 (s, 2H), 3.9 (s, 6H); m/z 301.

### Method 131

### 3-Benzyloxy-5-hydroxymethyl-benzoic acid methyl ester

A solution of 3-(benzyloxy)-5-(methoxycarbonyl)benzoic acid (Method 41; 4.5 g, 15.7 mmol) in THF (30 ml) was treated with BH₃-dimethyl sulfide (2.0 M in THF, 9.5 ml, 19 mmol) dropwise under nitrogen at 0° C. The mixture was stirred at 0° C. for 30 min then heated up to 60° C. for 6 h. The reaction was quenched with H₂O (5 ml) and the resulting mixture was concentrated under reduced pressure. The residue was then purified by column chromatography utilizing an ISCO system (EtOAc-Hexane) to give 3.73 g (87%) of colourless oil. NMR: δ 7.70 (s, 1H), 7.40-7.68 (m, 7H), 5.55 (t, 1H), 5.38 (s, 2H), 4.70 (d, 2H), 4.01 (s, 3H); m/z 273.

### Methods 132-135

The following compounds were prepared by the procedure of Method 131 using the appropriate SM and BH₃.

| Meth | Compound | m/z | SM |
|---|---|---|---|
| 132 | 3-Hydroxymethyl-5-methoxy-benzoic | 197 | Method 42 |
| | acid methyl ester | | |
| 133 | (3-Bromo-5-fluorophenyl)methanol | 206 | 3-Bromo-5- |
| | | | fluorobenzoic |
| | | | acid |
| 134 | 2-(3-Bromophenyl)-2-methylpropan-1-ol | 230 | Method 63 |
| 135 | Methyl 3-bromo-5- | 246 | Method 76 |
| | (hydroxymethyl)benzoate | | |

### Method 136

### 3-Benzyloxy-5-methanesulfonyloxymethyl-benzoic acid methyl ester

A solution of 3-benzyloxy-5-hydroxymethyl-benzoic acid methyl ester (Method 131; 3.73 g, 14 mmol) in DCM (20 ml) was cooled to 0° C. To this solution, triethylamine (4.2 g, 42 mmol, 3 eq) and methane sulfonyl chloride (3.19 g, 28 mmol, 2 eq) were added respectively. The mixture was stirred at 25° C. for 2 h. The resulting salts were removed by filtration and washed with DCM and hexane. The filtrate was concentrated under reduced pressure and then purified by column chromatography utilizing an ISCO system (EtOAc-hexane) to give 3.79 g of a colourless oil as the desired product (77%). NMR: δ 7.12-7.40 (m, 8H), 5.05 (s, 2H), 4.91 (s, 2H), 3.60 (s, 3H), 3.00 (s, 3H); m/z 351.

### Methods 137-139

The following compounds were prepared by the procedure of Method 136 using the appropriate SM and methane sulfonyl chloride.

| Meth | Compound | m/z | SM |
|---|---|---|---|
| 137 | 3-Methanesulfonyloxymethyl-5-methoxy-benzoic | 275 | Method |
| | acid methyl ester | | 132 |
| 138 | 3-Bromo-5-fluorobenzyl methanesulfonate | 284 | Method |
| | | | 133 |
| 139 | Methyl 3-bromo-5- | 324 | Method |
| | {[(methylsulfonyl)oxy]methyl}benzoate | | 135 |

### Method 140

### 3-(Cyano-dimethyl-methyl)-5-hydroxy-benzoic acid methyl ester

A suspension of 3-benzyloxy-5-(cyano-dimethyl-methyl)-benzoic acid methyl ester (Method 20; 1.7 g, 5.5 mmol) in MeOH (20 ml) was treated with 10% Pd on carbon (80 mg). The reaction was then placed on a Parr hydrogenator at 48 psi for 3 h. The reaction mixture was then filtered through celite and the solvents were removed under reduced pressure to give a white solid 1.2 g (100%). NMR: δ 7.60 (s, 1H), 7.36 (s, 1H), 7.20 (s, 1H), 3.88 (s, 3H), 1.72 (s, 6H); m/z 220.

### Method 141

### 3-(Cyano-dimethyl-methyl)-5-(2-dimethylamino-ethoxy)-benzoic acid methyl ester

A suspension of 3-(cyano-dimethyl-methyl)-5-hydroxy-benzoic acid methyl ester (Method 140; 500 mg, 2.283 mmol), 2-(dimethyl amino)ethyl chloride hydrochloride (427 mg, 2.97 mol, 1.3 eq), K₂CO₃ (3.15 g, 22.8 mmol, 10 eq) and sodium iodide (35 mg, 0.23 mmol, 0.1 eq) in acetone was heated to reflux for 5 h. The salt was removed by filtration, and the filtrate was concentrated to yield 662 mg (100%) of light yellow oil as desired product. NMR: δ 7.75 (s, 1H), 7.50 (s, 1H), 7.40 (s, 1H), 4.20 (t, 2H), 3.95 (s, 3H), 2.70 (t, 3H), 2.28 (s, 6H), 1.75 (s, 6H). m/z 290.

### Method 142

### 2-(5-Formyl-2-thienyl)-2-methylpropanenitrile

A solution of 2-methyl-2-(2-thienyl)propanenitrile (Method 22; 260 mg, 1.71 mmol) in THF (5.8 ml) was cooled to −78° C. To the cooled reaction was added 1.26 ml of tert-butyl lithium (1.7 M solution in pentanes) drop wise. The resulting bright yellow mixture was allowed to stir for 1 h before DMF (0.330 ml, 4.27 mmol) was added. The reaction was stirred for 6 h at −78° C. before being quenched by the addition of 25 ml of NH₄Cl(sat). The resulting mixture was extracted with EtOAc. The combined organic phase was washed with NaCl(sat), dried with MgSO₄(s), and concentrated under reduced pressure giving 271 mg of the title compound (88%) as a colourless oil; m/z 180.

### Method 143

The following compound was prepared by the procedure of Method 142 using the appropriate SM.

| Meth | Compound | m/z | SM |
|---|---|---|---|
| 143 | 4-({[tert-Butyl(diphenyl)silyl]oxy}methyl)- | 381 | Method |
| | thiophene-2-carbaldehyde | | 147 |

### Method 144.

### 5-(1-Cyano-1-methylethyl)thiophene-2-carboxylic acid

A solution of 2-(5-formyl-2-thienyl)-2-methylpropanenitrile (Method 142; 0.271 g, 1.51 mmol) in tertiary butyl alcohol (7.5 ml) and 2-methyl-2-butene (4.5 ml) was treated dropwise with an aqueous pre-mixed solution of NaClO₂ (1.22 g, 13.60 mmol) and NaH₂PO₄ (1.45 g, 10.57 mmol) in H₂O (7 ml). The reaction mixture was stirred for 30 min at 25° C. before the volatiles were removed under reduced pressure. The product was washed with NaHCO₃(sat) (1×50 ml) and extracted with EtOAc. The combined organic phase was washed with NaCl(sat) (50 ml), dried with MgSO₄(s), and concentrated under reduced pressure giving 0.265 g of the title compound (90%) as a white solid; m/z 196.

### Methods 145-146.

The following compounds were prepared by the procedure of Method 144 using the appropriate SM.

| Meth | Compound | m/z | SM |
|---|---|---|---|
| 145 | 4-({[tert-Butyl(diphenyl)silyl]oxy}methyl)- | 397 | Method |
| | thiophene-2-carboxylic acid | | 143 |
| 146 | 5-(1-Cyano-1-methylethyl)thiophene-3-carboxylic | 180 | Method |
| | acid | | 196 |

### Method 147

### tert-Butyl(diphenyl)(3-thienylmethoxy)silane

A solution of 3-thienylmethanol (5.0 g, 43.8 mmol) and imidazole (8.94 g, 131.4 mmol) in DMF (86 ml) was treated with tert-butylchlorodiphenylsilane (15.0 g, 54.7 mmol) at 0° C. The reaction stirred for 6 h at 25° C. before being quenched by the addition of 250 ml NH₄Cl(sat). The resulting mixture was extracted with EtOAc. The combined organic phase was washed once with NaCl(sat) (100 ml), dried with MgSO₄(s), and concentrated under reduced pressure. The crude reaction product was purified by column chromatography utilizing an ISCO system (hexanes-EtOAc, 10:1) giving 14.8 g of the title compound as a colourless oil (96%); m/z 353.

### Method 148

### Methyl 4-(hydroxymethyl)thiophene-2-carboxylate

A solution of 4-({[tert-butyl(diphenyl)silyl]oxy}methyl)thiophene-2-carboxylic acid (Method 145; 0.900 g, 2.27 mmol) in MeOH (50 ml) was treated with concentrated HCl (1.0 ml). The reaction was heated at reflux for 12 h and then concentrated under reduced pressure. The crude reaction product was washed with NaHCO₃(sat) (100 ml) and extracted with EtOAc. The organic phase was dried with MgSO₄(s) and concentrated under reduced pressure. The product was purified by column chromatography utilizing an ISCO system (hexanes-EtOAc, 3:1) giving 0.190 g of the title compound as a colourless oil (50%); m/z 173.

### Methods 149-151

The following compounds were prepared by the procedure of Method 148 using the appropriate starting materials.

| Meth | Compound | m/z | SM |
|---|---|---|---|
| 149 | Methyl (3-bromophenyl)acetate | 230 | (3-Bromophenyl)-acetic |
| | | | acid |
| 150 | Methyl 2-fluoro-5- | 169 | 2-Flouro-5-methyl |
| | methylbenzoate | | benzoic acid |
| 151 | Methyl 3-acetylbenzoate | 179 | 3-Acetylbenzoic acid |

### Method 152

### Methyl 4-(bromomethyl)thiophene-2-carboxylate

A solution of methyl 4-(hydroxymethyl)thiophene-2-carboxylate (Method 148; 0.191 g, 1.10 mmol) in THF (5 ml) was treated with phosphorous tribromide (0.357 g, 1.32 mmol). The reaction was stirred for 1 h at 25° C. before being quenched NaHCO₃(sat) (10 ml). The reaction mixture was extracted with EtOAc and the combined organic phase was dried with MgSO₄(s) and concentrated under reduced pressure. The product was purified by column chromatography utilizing an ISCO system (hexanes-EtOAc, 10:1) giving 0.155 g of the title compound as a yellow oil (60%); m/z 236.

### Method 153

The following compound was prepared by the procedure of Method 152 using the appropriate SM.

| Meth | Compound | m/z | SM |
|---|---|---|---|
| 153 | {[5-(Bromomethyl)-3- | 251 | [4-({[tert- |
| | thienyl]methoxy}(tert- | | butyl(diphenyl)silyl]oxy}methyl)- |
| | butyl)diphenylsilane | | 2-thienyl]methanol (Method 197) |

### Method 154

### 2-Methyl-2-(6-methylpyridin-2-yl)propanenitrile

A solution of 2-fluoro-6-methylpyridine (1.00 g, 9.00 mmol) and 2-methylpropanenitrile in toluene (30 ml) was treated with potassium hexamethyldisilazide (13.5 mmol) and the reaction was refluxed for 1 h before being cooled to 25° C. The reaction was then quenched with saturated aqueous NH₄Cl (50 ml) and the mixture was extracted with EtOAc. The combined organic phase was dried with MgSO₄(s) and concentrated under reduced pressure. The product was purified by column chromatography utilizing an ISCO system (hexanes-EtOAc, 5:1) giving 0.990 g of the title compound as a colourless oil (70%); m/z 162.

### Method 155

### 6-(1-Cyano-1-methylethyl)pyridine-2-carboxylic acid

A solution of 2-methyl-2-(6-methylpyridin-2-yl)propanenitrile (Method 154; 0.850 g, 5.30 mmol) in pyridine (50 ml) was treated with selenium dioxide (2.64 g, 23.87 mmol). The reaction was heated to reflux for 72 h. After this time, the pyridine was removed by distillation and the resulting residue was washed with EtOAc (200 ml) and H₂O (100 ml). The organic phase was washed with 1N HCl and then NaCl(sat). The organic phase was dried with MgSO₄(s) and, concentrated under reduced pressure. The product was purified by column chromatography utilizing an ISCO system (EtOAc-MeOH, 10:1) giving 0.313 g of the title compound as a white solid (32%) m/z 191.

### Method 156

### Methyl 3-(bromomethyl)-4-chlorobenzoate

A solution of methyl 4-chloro-3-methylbenzoate (2.50 g, 13.54 mmol) and N-bromosuccinimide (3.00 g, 16.93 mmol) in carbon tetrachloride (50 ml) was treated with azobisisobutyronitrile (500 mg. The solution was heated to 80° C. for 4 h before being cooled to 25° C. The reaction mixture was filtered through a pad of celite and the filtrate was concentrated under reduced pressure. The product was purified by column chromatography utilizing an ISCO system (hexanes-EtOAc, 10:1) giving 2.70 g of the title compound as a white solid (76%); m/z 264.

### Methods 157-168

The following compounds were prepared by the procedure of Method 156 using the appropriate SM.

| Meth | Compound | m/z | SM |
|---|---|---|---|
| 157 | Ethyl 2-(bromomethyl)-1,3-thiazole-4- | 251 | Ethyl 2-(methyl)-1,3-thiazole-4- |
| | carboxylate | | carboxylate |
| 158 | Methyl 4-(bromomethyl)-3- | 298 | Methyl 4-methyl-3- |
| | (trifluoromethyl)benzoate | | (trifluoromethyl)benzoate |
| 159 | Methyl 5-(bromomethyl)nicotinate | 231 | Methyl 5-methylnicotinate |
| 160 | Methyl 3-(bromomethyl)-1-methyl-1H- | 234 | Methyl 1,3-dimethyl-1H-pyrazole- |
| | pyrazole-5-carboxylate | | 5-carboxylate |
| 161 | Methyl 5-(bromomethyl)-1-methyl-1H- | 234 | Methyl 1,5-dimethyl-1H-pyrazole- |
| | pyrazole-3-carboxylate | | 3-carboxylate |
| 162 | Methyl 5-(bromomethyl)-2-furoate | 220 | Methyl 5-methyl-2-furoate |
| 163 | Methyl 5-(bromomethyl)isoxazole-3- | 221 | Methyl 5-methylisoxazole-3- |
| | carboxylate | | carboxylate |
| 164 | Methyl 5-(bromomethyl)-2- | 248 | Method 150 |
| | fluorobenzoate | | |
| 165 | 4-Bromomethyl-3-trifluoromethyl- | 297 | Method 211 |
| | benzoic acid methyl ester | | |
| 166 | Methyl 3-(bromomethyl)-5- | 244 | Methyl 3,5-dimethylbenzoate |
| | methylbenzoate | | |
| 167 | Methyl 3-(bromomethyl)-5-(1-cyano-1- | 297 | Method 38 |
| | methylethyl)benzoate | | |
| 168 | Methyl 3-(bromomethyl)-4- | 248 | Method 210 |
| | fluorobenzoate | | |

### Method 169

### 3-[(Dimethylamino)sulfonyl]benzoic acid

A solution of 3-(chlorosulfonyl)benzoic acid (2.60 g, 12 mmol) in DCM (20 ml) was treated with dimethylamine (2.0 M in THF, 20 ml, 40 mmol, 3.3 equiv). After 30 min, the reaction was quenched with 10% HCl and extracted with EtOAc. The organics were washed with NaCl(sat) and then dried with Na₂SO₄(s). The organics were then removed under reduced pressure to give 1.80 g, 65%; m/z 229.

### Methods 170-179

The following compounds were prepared by the procedure of Method 169, using the appropriate starting material.

| Meth | Compound | m/z | SM |
|---|---|---|---|
| 170 | 3-[(Cyclopropylamino)sulfonyl]benzoic acid | 241 | Cyclopropylamine |
| 171 | 3-(Aminosulfonyl)benzoic acid | 202 | Ammonia |
| 172 | 3-{[4-(Hydroxymethyl)piperidin-1- | 300 | Piperidin-4- |
| | yl]sulfonyl}benzoic acid | | ylmethanol |
| 173 | 3-{[3-(Hydroxymethyl)piperidin-1- | 300 | Piperidin-3- |
| | yl]sulfonyl}benzoic acid | | ylmethanol |
| 174 | 3-{[2-(Hydroxymethyl)piperidin-1- | 300 | Piperidin-2- |
| | yl]sulfonyl}benzoic acid | | ylmethanol |
| 175 | 3-{[Methoxy(methyl)amino]sulfonyl}benzoic acid | 246 | (Methoxyamino)- |
| | | | methane |
| 176 | 3-{[(2,3-Dihydroxypropyl)(methyl)amino]sulfonyl} | 304 | 3-(Methylamino) |
| | benzoic acid | | propane-1,2-diol |
| 177 | 3-{[(Tetrahydrofuran-2- | 286 | (Tetrahydrofuran-2- |
| | ylmethyl)amino]sulfonyl}benzoic acid | | ylmethyl)amine |
| 178 | 3-(Morpholin-4-ylsulfonyl)benzoic acid | 272 | Morpholine |
| 179 | 3-(Azetidin-1-ylsulfonyl)-benzoic acid | 241 | Azetidine |

### Method 180

### Methyl 4-{[(tert-butoxycarbonyl)(cyclopropyl)amino]methyl}-3-(trifluoromethyl)benzoate

A mixture of methyl 4-[(cyclopropylamino)methyl]-3-(trifluoromethyl)benzoate (Method 234; 0.80 g, 0.29 mmol), di-tert-butyl dicarbonate (0.70 g, 0.32 mmol) and K₂CO₃ (1.21 g, 0.87 mmol) was stirred in THF (12 ml) and water (4 ml) at 25° C. for 4.5 h and the solvents were removed under reduced pressure. The crude residue was taken up in EtOAc, washed with water, NaCl(sat), dried, filtered and concentrated under reduced pressure. Purification by chromatography (SiO₂) afforded the desired product; m/z 374.

### Method 181

### 6-Bromo-3-methyl-2-thioxo-2,3-dihydroquinazolin-4(1H)-one

A solution of 2-amino-5-bromobenzoic acid (2.0 g, 9.26 mmol) and methyl isothiocyanate (0.63 ml, 9.26 mmol) in acetic acid (20 ml) was stirred at 120° C. for 3 h. The reaction mixture was concentrated under reduced pressure. The residue was taken up in diethyl ether, filtered and washed twice with diethyl ether to give 1.47 g (59%); m/z 272.

### Method 182

### 6-Bromo-3-methyl-2-(methylthio)quinazolin-4(3H)-one

Iodomethane (0.51 ml, 8.13 mmol) was added to a stirring solution of 6-bromo-3-methyl-2-thioxo-2,3-dihydroquinazolin-4(1H)-one (Method 181; 1.47 g, 5.42 mmol) in 1 N sodium hydroxide (20 ml) and acetone (50 ml) was stirred at 25° C. for 30 min. The resultant solids were collected by vacuum filtration and washed with diethyl ether; m/z 286.

### Method 183

### 2-Methyl-2-(4-methylpyridin-2-yl)propanenitrile

A 100 ml round bottom flask fitted with a reflux condenser was charged with 2-fluoro-4-methylpyridine (1.00 g, 9.00 mmol), 2-methylpropanenitrile (2.48 g, 36 mmol), and toluene (30 ml). Potassium Hexamethyldisilazide (13.5 mmol) was added and the reaction was refluxed for 1 h. before being cooled to 25° C. The reaction was then quenched with NH₄Cl(sat) (50 ml) and the mixture was extracted with EtOAc (2×50 ml). The combined organic phase was dried with MgSO₄ and concentrated in vacuo to yield the crude reaction product which was purified on 40 g SiO₂ hexanes-EtOAc (5:1) as eluent giving 0.870 g of the title compound as a colourless oil (60%); m/z 161.

### Method 184

### 2-(1-Cyano-1-methylethyl)isonicotinic acid

A 50 ml three neck flask equipped with a reflux condenser was charged with 2-methyl-2-(4-methylpyridin-2-yl)propanenitrile (Method 183; 0.870 g, 5.43 mmol), and water (15 ml). The reaction mixture was heated to 60° C. and KMnO₄ (4.3 g, 27 mmol) was added. The reaction was heated to reflux for 2 h, and was then filtered through a bed of Celite. The pH was adjusted to 4 by the careful addition of 1N HCl and the aqueous phase was extracted with EtOAc (4×25 ml). The organic phase was dried with MgSO₄ and concentrated in vacuo to yield the crude reaction product which was purified on 40 g SiO₂ using EtOAc-MeOH (10:1) as eluent giving 0.700 g of the title compound as a white solid (68%); m/z 191.

### Methods 185-186

The following compounds were prepared by the procedure of Method 184, using the appropriate starting material.

| Meth | Compound | m/z | SM |
|---|---|---|---|
| 185 | 3-(1-Cyano-1-methylethyl)-2- | 208 | Method 37 |
| | fluorobenzoic acid | | |
| 186 | 3-(1-Carboxy-1-methylethyl)-2- | 227 | Method 37 |
| | fluorobenzoic acid¹ | | |
| 187 | 3-tert-Butylbenzoic acid | 179 | 1-tert-Butyl-3- |
| | | | methylbenzene |

¹Formed as a by-product of Method 185

### Method 188

### Ethyl 3-(3,3-dimethylbut-1-yn-1-yl)benzoate

Ethyl 3-bromobenzoate (0.500 g, 2.18 mmol) was dissolved in CH₃CN (8.70 ml). Triethylamine (1.53 ml, 10.9 mmol) was added followed by 3,3-dimethylbut-1-yne (0.27 g, 3.27 mmol). With stirring Pd(PPh₃)₄ (0.25 g, 0.21 mmol) and CuI (0.083 g, 0.436 mmol) were added and the reaction was warmed to 60° C. for 4 h. The reaction was then diluted with EtOAc (˜50 ml), filtered through a pad of SiO₂, and concentrated in vacuo. The crude product was purified on 40 g SiO₂ using hexanes-EtOAc (10:1) as eluent giving 0.45 g of the title compound as a colourless oil (91%); m/z 231.

### Methods 189-191

The following compounds were prepared by the procedure of Method 188, using the appropriate starting materials.

| Meth | Compound | m/z | SM |
|---|---|---|---|
| 189 | Ethyl 3-(3-hydroxy-3-methylbut-1- | 233 | 2-Methylbut-3-yn-2-ol |
| | yn-1-yl)benzoate | | and ethyl 3- |
| | | | bromobenzoate |
| 190 | Ethyl 3- | 215 | Ethynylcyclopropane |
| | (cyclopropylethynyl)benzoate | | and ethyl 3- |
| | | | bromobenzoate |
| 191 | Methyl 3-(1-cyano-1-methylethyl)- | 258 | Prop-2-yn-1-ol and |
| | 5-(3-hydroxyprop-1-yn-1- | | Method 39 |
| | yl)benzoate | | |

### Method 192

### Methyl 5-piperidin-1-ylnicotinate A 25 ml round bottom flask was charged with methyl 5-bromonicotinate (0.500 g, 2.31 mmol), piperidine (0.305 g, 3.46 mmol), and toluene (5 ml). Caesium carbonate (2.25 g, 6.93 mmol), palladium (II) acetate (52 mg, 0.23 mmol), and BINAP (0.287 g, 0.46 mmol) were then added. The reaction was heated to 80° C. for 8 h before being diluted with EtOAc (˜50 ml), filtered through a pad of SiO₂, and concentrated in vacuo. The crude product was purified on 40 g SiO₂ using EtOAc as eluent giving 0.376 g of the title compound as a colourless oil (74%); m/z 221.

### Methods 193-194

The following compounds were prepared by the procedure of Method 192, using the appropriate starting material.

| Meth | Compound | m/z | SM |
|---|---|---|---|
| 193 | Methyl 3-piperidin-1-ylbenzoate | 220 | Piperidine |
| 194 | Methyl 5-morpholin-4-ylnicotinate | 223 | Morpholine |

### Method 195

### 2-[4-(Hydroxymethyl)-2-thienyl]-2-methylpropanenitrile

THF (25 ml) was added to 2-[4-({[tert-butyl(diphenyl)silyl]oxy}methyl)-2-thienyl]-2-methylpropanenitrile (Method 31; 0.880 g, 2.10 mmol). A 1 M solution of tetrabutylammonium fluoride in THF (5.25 mmol) was added dropwise via syringe and the reaction was allowed to stir for 12 h at 25° C. before being quenched with NH₄Cl(sat) (50 ml). The reaction mixture was extracted with EtOAc (2×50 ml) and the combined organic phase was dried with MgSO₄ and concentrated in vacuo to yield the crude reaction product which was purified on 40 g SiO₂ using hexanes-EtOAc (2:1) as eluent giving 0.270 g of the title compound as a colourless oil (71%). m/z 182.

### Method 196

### 2-(4-Formyl-2-thienyl)-2-methylpropanenitrile

To DMSO (0.277 g, 3.55 mmol) was added 10 ml of DCM. The reaction was cooled to −78° C. and oxalyl chloride (0.225 g, 1.78 mmol) was added dropwise via syringe and the reaction was allowed to stir for 30 min at this temperature. A 1 M solution of 2-[4-(hydroxymethyl)-2-thienyl]-2-methylpropanenitrile (Method 195; 0.270 g, 1.48 mmol) in DCM was then added dropwise via syringe and the reaction was allowed to stir for 30 min at this temperature. Triethylamine (0.718 g, 7.40 mmol) was then added and the reaction was allowed to warm to 25° C. with stirring over 1 h before being quenched with NaHCO₃(sat) (250 ml). The reaction mixture was then extracted with EtOAc (2×50 ml) and the combined organic phase was dried with MgSO₄ and concentrated in vacuo to yield the crude reaction.

### Method 197

### [4-({[tert-Butyl(diphenyl)silyl]oxy}methyl)-2-thienyl]methanol

4-({[tert-Butyl(diphenyl)silyl]oxy}methyl)thiophene-2-carbaldehyde (Method 143; 3.99 g, 10.48 mmol) was dissolved in MeOH (50 ml). With stirring, NaBH₄ (0.792 g, 20.96 mmol) was added in one portion. After 1 h, the reaction was carefully quenched with a solution of NH₄Cl(sat) (˜250 ml). The resulting mixture was extracted with EtOAc (3×125 ml). The combined organic phase was washed with NaCl(sat) (250 ml), dried with MgSO₄, and concentrated in vacuo giving the crude reaction product which was purified on 120 g SiO₂ using hexanes-EtOAc (5:2) as eluent giving 3.99 g of the title compound as a colourless oil (98%). m/z 384.

### Method 198

### 3-(Cyano-dimethyl-methyl)-5-hydroxy-N-(4-methyl-3-nitro-phenyl)-benzamide

A solution of 3-(cyano-dimethyl-methyl)-5-methoxy-N-(4-methyl-3-nitro-phenyl)-benzamide (Method 80; 353 mg, 1 mmol) in 1M BBr₃ in DCM (5 ml) was stirred at 25° C. for 1 h. Crushed ice was then slowly added to the mixture, and then 1N NaOH was added to adjust the pH to 10. The organic layer was then separated and discarded. The water layer was then acidified with 10% HCl aq to pH 1˜3, and the resulting solid was collected by vacuum filtration to give 311 mg (91.7%) of the title compound. NMR: 10.45 (s, 1H), 10.00 (s, br, 1H), 8.41 (s, 1H), 7.95 (d, 1H), 7.40 (m, 2H), 7.25 (s, 1H), 7.08 (s, 1H), 2.45 (s, 3H), 1.65 (s, 6H); m/z 339.

### Method 199

### 3-(Cyano-dimethyl-methyl)-5 methylcarbamoylmethoxy-N-(4-methyl-3-nitro-phenyl)-benzamide

A suspension of 3-(cyano-dimethyl-methyl)-5-hydroxy-N-(4-methyl-3-nitro-phenyl)-benzamide (Method 198; 180 mg, 0.53 mmol), 2-chloro-N-methyl acetamide (68 mg, 0.64 mmol), K₂CO₃ (731 mg, 5.3 mmol) and sodium iodide (80 mg, 0.53 mmol) in 10 ml of acetone and 1,4-dioxane (1:1) was heated to reflux for 4 h. The resulting salt was filtered and washed with acetone. The filtrate and washings were concentrated under reduced pressure, and the residue was purified with an ISCO system (hexane-EtOAc), to give 169 mg (77.9%) of the title compound as white solid. NMR: 10.55 (s, 1H), 8.42 (s, 1H), 8.15 (s, br, 1H), 7.96 (d, 1H), 7.70 (s, 1H), 7.50 (m, 2H), 7.35 (s, 1H), 4.55 (s, 2H), 3.29 (s, 3H), 2.68 (d, 3H), 1.70 (s, 6H); m/z 410.

### Methods 200-205

The following compounds were prepared by the procedure of Method 199, using the appropriate starting material and 3-(cyano-dimethyl-methyl)-5-hydroxy-N-(4-methyl-3-nitro-phenyl)-benzamide (Method 198)

| Meth | Compound | m/z | SM |
|---|---|---|---|
| 200 | 3-(1-Cyano-1-methylethyl)-N-(4-methyl-3- | 454 | 4-(2-Chloroethyl)morpholine |
| | nitrophenyl)-5-(2-morpholin-4- | | |
| | ylethoxy)benzamide | | |
| 201 | 3-(1-Cyano-1-methylethyl)-N-(4-methyl-3- | 452 | 1-(2-Chloroethyl)piperidine |
| | nitrophenyl)-5-(2-piperidin-1- | | |
| | ylethoxy)benzamide | | |
| 202 | 3-(1-Cyano-1-methylethyl)-N-(4-methyl-3- | 480 | 1-(3-Chloropropyl)-4- |
| | nitrophenyl)-5-[3-(4-methylpiperazin-1- | | methylpiperazine |
| | yl)propoxy]benzamide | | |
| 203 | 3-(1-Cyano-1-methylethyl)-N-(4-methyl-3- | 451 | 2-(2-Chloroethyl)-1- |
| | nitrophenyl)-5-[2-(1-methylpyrrolidin-2- | | methylpyrrolidine |
| | yl)ethoxy]benzamide | | |
| 204 | 3-(2-Azepan-1-ylethoxy)-5-(1-cyano-1- | 465 | 1-(2-Chloroethyl)azepane |
| | methylethyl)-N-(4-methyl-3- | | |
| | nitrophenyl)benzamide | | |
| 205 | 3-(1-Cyano-1-methylethyl)-5-(2- | 398 | 1-Chloro-2-methoxyethane |
| | methoxyethoxy)-N-(4-methyl-3- | | |
| | nitrophenyl)benzamide | | |

### Method 206

### Benzyl 4-(iodomethyl)piperidine-1-carboxylate

Triphenylphosphine (7.87 g, 30 mmol) and imidazole (2.05 g, 30 mmol, 1.5 equiv) in DCM at 0° C. under Ar was treated with I₂ (7.61 g, 30 mmol, 1.5 equiv). After 5 min, benzyl 4-(hydroxymethyl)tetrahydro-1(2H)-pyridinecarboxylate (5.00 g, 20 mmol) in DCM was added. The reaction was stirred for 1 h and then quenched with 10% HCl. The reaction mixture was extracted with EtOAc and the organic layer was washed with NaHCO₃(sat). The organics were dried with NaCl(sat) and Na₂SO₄(s) and then removed under reduced pressure. The residue was then purified by column chromatography utilizing an ISCO system (EtOAc-hexane) to give 6.20 g (86%) of a white solid; m/z 360.

### Method 207

The following compound was prepared by the procedure of Method 206, using the appropriate starting materials

| Meth | Compound | m/z | SM |
|---|---|---|---|
| 207 | tert-Butyl | 286 | tert-Butyl (3- |
| | (3-iodopropyl)carbamate | | hydroxypropyl)carbamate |

### Method 208

### 3-(1-Cyano-1-methylethyl)-5-fluorobenzoic acid

2-(3-Bromo-5-fluorophenyl)-2-methylpropanenitrile (Method 32; 258 mg, 1.07 mmol) in THF (10 ml) at −78° C. under Ar was treated with t-BuLi (1.7 M in pentane, 2.13 mmol, 2.0 equiv). The reaction stirred for 15 min and then CO_{2(g)} was bubbled through the reaction mixture. After 10 min, the reaction was quenched with 10% NaOH and extracted with EtOAc. The aqueous layer was acidified with 10% HCl and extracted with EtOAc. The organics were dried with NaCl(sat) and Na₂SO₄(s) and then removed under reduced pressure; m/z 208.

### Method 209

The following compound was prepared by the procedure of Method 208, using the appropriate starting material.

| Meth | Compound | m/z | SM |
|---|---|---|---|
| 209 | 3-(2-Methoxy-1,1-dimethylethyl)benzoic acid | 209 | Method 34 |

### Method 210

### Methyl 4-fluoro-3-methylbenzoate

To a stirring solution of 4-fluoro-3-methylbenzoic acid (5.0 g, 0.032 mol) and K₂CO₃ (9.0 g 0.064 mol) in 80 ml DMF was added iodomethane (2.4 ml, 0.038 mol). The reaction mixture was allowed to stir at 25° C. for 15 h. The DMF was removed under reduced pressure and the resulting residue was washed with EtOAc and H₂O. The organic layer was dried and the solvent was removed under reduced pressure. m/z 169.

### Method 211

The following compounds were prepared by the procedure of Method 210, using the appropriate starting material.

| Meth | Compound | m/z | SM |
|---|---|---|---|
| 211 | 4-Methyl-3-trifluoromethyl-benzoic acid | 218 | 4-Methyl-3- |
| | methyl | | trifluoromethyl- |
| | | | benzoic acid |

### Method 212

### 4-Iodo-1-methylpiperidine

4-Chloro-1-methyl-piperidine hydrochloride (4 g, 23.5 mmol) was dissolved in 40 ml of K₂CO₃ solution. The solution was extracted with EtOAc (3×50 ml). The combined extracts were dried and concentrated under reduced pressure to about 50 ml. NaI (3.55 g, 23.7 mmol) was then added to the solution and the suspension was stirred at 25° C. for 30 min. Water was added and the organic layer separated and dried. The organics were concentrated to give the title compound as a yellow oil.

### Method 213

### 3-(Cyano-dimethyl-methyl)-N-(4-methyl-3-nitro-phenyl)-5-(1-methyl-piperidin-4-yloxy)-benzamide

To a cooled suspension of NaH (60% dispersed in mineral oil) (32 mg, 0.79 mmol) in DMF (4 ml) was added dropwise a solution of 3-(cyano-dimethyl-methyl)-5-hydroxy-N-(4-methyl-3-nitro-phenyl)-benzamide (Method 198; 268 mg, 0.79 mmol) in DMF (2 ml). Then 4-iodo-1-methyl-piperidine (Method 212; 178 mg, 0.79 mmol) in DMF (2 ml) was added. The reaction mixture was heated to reflux for 12 h. After cooling to 25° C., water (20 ml) was added to the mixture. The resulting solution was extracted with EtOAc (3×30 ml). The combined extracts were dried and concentrated under reduced pressure. The resulting residue was then purified by a Gilson HPLC (0.1% TFA in acetonitrile and water) to yield 60 mg (17%) of the title compound; m/z 436.

### Method 214

### 4-Dimethylaminomethyl-3-trifluoromethyl-benzoic acid methyl ester

A mixture of 4-bromomethyl-3-trifluoromethyl-benzoic acid methyl ester (Method 165; 400 mg, 1.35 mmol), dimethyl amine (2.0 M in THF) (2 ml, 4 mmol) and K₂CO₃ (373 mg, 2.7 mmol) in CH₃CN (10 ml) was stirred at 25° C. for 1 h. The temperature was then raised to 80° C. over 1 h and stirred at this temperature for 3 h. The reaction mixture was cooled to 25° C. and washed with DCM. The organics were concentrated under reduced pressure, and the resulting residue was purified by column chromatography utilizing an ISCO system (hexane-EtOAc) to yield the title compound as a colourless oil 230 mg (65.3%). NMR: 8.25 (d, 1H), 8.20 (s, 1H), 7.95 (d, 1H), 3.90 (s, 3H), 3.60 (s, 2H), 2.18 (s, 6H); m/z 261.

### Methods 215-216

The following compounds were prepared by the procedure of Method 214, using the appropriate starting materials

| Meth | Compound | m/z | SM |
|---|---|---|---|
| 215 | Methyl 3-(1-cyano-1-methylethyl)-5-[(4- | 315 | Method 167 |
| | methylpiperazin-1-yl)methyl]benzoate | | and N-methyl |
| | | | piperazine |
| 216 | Methyl 3-(1-cyano-1-methylethyl)-5- | 260 | Method 167 |
| | [(dimethylamino)methyl]benzoate | | and |
| | | | dimethylamine |

### Method 217

### Methyl 3-[3-(trimethylsilyl)prop-2-yn-1-yl]benzoate

Trimethylsilyl acetylene (2.4 ml, 17.0 mmol) was added to a solution of methyl 3-(bromomethyl)benzoate (3.0 g, 13.1 mmol), Pd₂ dba₃ (300 mg, 0.3 mmol), triphenylphosphine (343 mg, 1.3 mmol), Cs₂CO₃ (6.0 g, 18.3 mmol), and CuI (187 mg, 1.0 mmol) in THF (50 ml). The reaction mixture was stirred for 12 h at 50° C. After allowing the mixture to cool back to 25° C., it was then diluted with EtOAc (˜100 ml) and washed with NaCl(sat). The mixture was then filtered through a pad of celite, dried and concentrated in vacuo. The crude product was purified on SiO₂ using hexanes-EtOAc 4:1 as eluent giving 2.2 g (67%) as product. H NMR (300 MHz): 8.03 (s, 1H), 7.92 (d, 1H), 7.57 (d, 1H), 7.40 (t, 1H), 3.93 (s, 3H), 3.71 (s, 2H), 0.21 (s, 9H).

### Method 218

### Methyl 3-[1,1-dimethyl-3-(trimethylsilyl)prop-2-yn-1-yl]benzoate

A solution of methyl 3-[3-(trimethylsilyl)prop-2-yn-1-yl]benzoate (Method 217; 350 mg, 1.28 mmol) in THF (6 ml) was treated with NaHMDS (2.8 ml, 2.81 mmol), at −78° C. Iodomethane (0.2 ml) was added and the reaction mixture was warmed to 25° C. and stirred for an additional 2 hr. The reaction mixture was then quenched with NH₄Cl(sat) solution and extracted with EtOAc. The combined organics were dried and concentrated under reduced pressure. The crude product was purified by column chromatography utilizing an ISCO system (hexane-EtOAc) to give 200 mg (52%) of desired product. H NMR (300 MHz): 8.25 (s, 1H), 7.91 (d, 1H), 7.78 (d, 1H), 7.40 (t, 1H), 3.92 (s, 3H), 1.62 (s, 6H), 0.23 (s, 9H).

### Method 219

### 3-(1,1-Dimethylprop-2-yn-1-yl)benzoic acid

To a solution of methyl 3-[1,1-dimethyl-3-(trimethylsilyl)prop-2-yn-1-yl]benzoate (Method 218; 110 mg, 0.36 mmol) in a solvent system of THF (4 ml), MeOH (2 ml) and H₂O (2 ml) was added lithium hydroxide (26 mg, 1.09 mmol) and the reaction mixture was stirred at 25° C. for 12 h. The reaction mixture was diluted with EtOAc and water. The aqueous layer was separated and then was acidified with 10% HCl and subsequently extracted with EtOAc. The combined extracts were dried to give 60 mg (88%) of desired product; m/z 188.

### Method 220

### 3-(1,1-Dimethylpropyl)benzoic acid

3-(1,1-Dimethylprop-2-yn-1-yl)benzoic acid (Method 219; 170 mg, 0.90 mmol) in MeOH (5 ml) was treated with Pd/C (17 mg). The reaction mixture was stirred for 12 h under an atmosphere of Hydrogen gas at 25° C. The mixture was filtered through celite, and the solvent was removed under reduced pressure to yield the desired product (150 mg, 86%); m/z 192.

### Method 221

### Ethyl 3-(cyclopropylcarbonyl)benzoate

To a solution of ethyl 3-iodobenzoate (1.8 ml, 10.0 mmol) in THF (40 ml) at −78° C., isopropyl magnesium chloride (2.0M, 7.0 ml, 14.0 mmol) was added. After 30 mins of stirring, CuCN (1.1 g, 12.0 mmol) and LiCl (1.0 g, 24.0 mmol) were added simultaneously. After 20 min, cyclopropane carbonyl chloride (3.0 ml, 33.0 mmol) was added, and then the reaction mixture was warmed to 25° C. over 1 h. The mixture was diluted with EtOAc and washed sequentially with NH₄Cl(sat) and NaCl(sat). The organics were dried, and the solvents removed under reduced pressure. The crude product was purified by column chromatography utilizing an ISCO system (hexane-EtOAc) to yield 1.2 g (50%). H NMR (300 MHz): 8.66 (s, 1H), 8.22 (d, 1H), 8.17 (d, 1H), 7.55 (t, 1H), 4.40 (q, 2H), 2.76-2.67 (m, 1H), 1.40 (t, 3H), 1.29-1.21 (m, 2H), 1.12-1.01 (m, 2H).

### Method 222

### Ethyl 3-(1-cyclopropyl-1-hydroxyethyl)benzoate

To a solution of ethyl 3-(cyclopropylcarbonyl)benzoate (Method 221; 363 mg, 1.66 mmol) in THF (6 ml) at −78° C., methyl magnesium bromide (3.0M, 0.73 ml, 2.16 mmol) was added. After 3 h, the mixture was diluted with EtOAc and then washed sequentially with NH₄Cl(sat) and then NaCl(sat). The organics were dried, and the resulting material was purified by column chromatography utilizing an ISCO system (hexane-EtOAc) to yield 1.2 g (50%) of the desired product. H NMR (300 MHz): 8.19 (s, 1H), 7.92 (d, 1H), 7.72 (d, 1H), 7.40 (t, 1H), 4.37 (q, 2H), 1.78 (s, 1H), 1.51 (s, 3H), 1.38 (t, 3H), 1.32-1.21 (m, 1H), 0.46-0.37 (m, 4H).

### Method 223

### Ethyl 3-[cyclopropyl(hydroxy)methyl]benzoate

To a solution of ethyl 3-(cyclopropylcarbonyl)benzoate (Method 221; 363 mg, 1.66 mmol) in EtOH (5 ml) at 25° C., NaBH₄ (70 mg, 1.86 mmol) was added. After 4 h, the mixture was diluted with EtOAc and then washed sequentially with NH₄Cl(sat) and then NaCl(sat). The organics were dried, and the resulting material was purified by column chromatography utilizing an ISCO system (hexane-EtOAc) to yield 210 mg (77%) of the desired product. H NMR (300 MHz): 8.07 (s, 1H), 7.95 (d, 1H), 7.61 (d, 1H), 7.41 (t, 1H), 4.36 (q, 2H), 4.04 (d, 1H), 2.16 (s, 1H), 1.38 (t, 3H), 1.27-1.15 (m, 1H), 0.66-0.54 (m, 2H), 0.52-0.36 (m, 2H).

### Method 224

### Methyl 3-(1,1-difluoroethyl)benzoate

A solution of methyl 3-acetylbenzoate (Method 151; 700 mg, 3.9 mmol) in 5 ml of DeoxoFluor™ was stirred for 12 h at 85° C. The reaction mixture was then added to a NaCl(sat) solution. The aqueous mixture was extracted with EtOAc. The organics were dried, and the resulting material was purified by column chromatography utilizing an ISCO system (hexane-EtOAc) to yield a clear oil (396 mg, 50%). H NMR (300 MHz): 7.96 (s, 1H), 7.86 (d, 1H), 7.50 (d, 1H), 7.31-7.22 (m, 1H), 3.73 (s, 3H), 1.74 (t, 3H).

### Method 225

### Sodium [3-(1-cyano-1-methylethyl)-5-(methoxycarbonyl)phenyl]methanesulfonate

A solution of methyl 3-(bromomethyl)-5-(1-cyano-1-methylethyl)benzoate (Method 167; 230 mg, 0.777 mmol) in acetone (5 ml) and water (5 ml) was added sodium sulfite. The mixture was stirred at reflux. The solvents were removed under reduced pressure to give the product; m/z 297.

### Method 226

### Methyl 3-(1-cyano-1-methylethyl)-5-[(methylthio)methyl]benzoate

A solution of methyl 3-(bromomethyl)-5-(1-cyano-1-methylethyl)benzoate (Method 167; 80 mg, 0.27 mmol) in EtOH (1 ml) was added sodium sulfite. The mixture was stirred at reflux. The solvents were removed under reduced pressure to give the product; m/z 263.

### Method 227

### Methyl 3-(1-cyano-1-methylethyl)-5-[3-(4-methylpiperazin-1-yl)prop-1-yn-1-yl]benzoate

To a solution of methyl 3-(1-cyano-1-methylethyl)-5-(3-hydroxyprop-1-yn-1-yl)benzoate (Method 191; 115 mg, 0.447 mmol) and triethylamine (81 μL, 0.581 mmol) in DCM was added methane sulfonyl chloride (52 μL, 0.671 mmol). The reaction mixture was allowed to stir for 15 min at 25° C. The solvent was removed under reduced pressure and the residue was dissolved in EtOAc. The organics were washed with NaCl(sat) and then dried. The solvents were removed under reduced pressure to provide 149 mg (quantitative yield) of the desired intermediate. The material was then dissolved in DCM (3 ml). Triethylamine (190 μL, 1.34 mmol) and K-methyl piperazine were then added to the mixture and stirred for 12 h. The solvents were removed under reduced pressure and the resulting material was purified by column chromatography utilizing an ISCO system (DCM-MeOH) to yield 50 mg (33%) of desired product; m/z 339.

### Method 228

### N-Cyclopropylformamide

Cyclopropylamine (5.0 ml, 72 mmol) and methyl formate (4.5 ml, 72 mmol) were added together and heated to reflux. After 12 h, the excess starting materials were removed under reduced pressure and the material was utilized directly.

### Method 229

### tert-Butyl (4-methyl-3-nitrophenyl)carbamate

A solution of 4-methyl-3-nitroaniline (10.0 g, 0.066 mol) was dissolved in THF (25 ml) at 65° C. Di-tert-butyl dicarbonate (17.2 g, 0.079 mol, 1.2 equiv) in THF (20 ml) was added dropwise over 30 min. The mixture was then refluxed under nitrogen for 12 h. The reaction was cooled to 25° C. and the solvent was removed under reduced pressure to give a brown oil. The oil was dissolved in hexane-EtOAc (4:1) and 30 g of silica gel was added to the solution. The solution was stirred for 5 min and the silica was removed by filtration. The silica was then repeatedly washed with hexane-EtOAc (4:1) until no further product was detected. The solvents were combined and concentrated under reduced pressure. The resulting yellow solid was washed with hexane and air dried to give 14.2 g of the desired product (85%). NMR (300 MHz): 8.07 (s, 1H), 7.53 (d, 1H), 7.26-7.30 (m, 1H), 6.66 (s, 1H), 2.55 (s, 3H), 1.55 (s, 9H).

### Method 230

### tert-Butyl (3-amino-4-methylphenyl)carbamate

A solution of tert-butyl (4-methyl-3-nitrophenyl)carbamate (Method 229; 10.0 g, 39.6 mmol) was dissolved in EtOH (220 ml). The solution was treated with 10% Pd/C (650 mg) and placed on a Parr Hydrogenator at 50 psi of hydrogen for 12 h. The resulting solution was filtered through celite and the solvent was removed under reduced pressure to give 8.68 g (98%). NMR (300 MHz): 6.86-6.98 (m, 2H), 6.48 (d, 1H), 6.36 (s, 1H), 3.59 (s, 2H), 2.09 (s, 3H), 1.42-1.50 (m, 9H).

### Method 231

### tert-Butyl {4-methyl-3-[(3-methyl-4-oxo-3,4-dihydroquinazolin-6-yl)amino]phenyl}carbamate

A stirred mixture of tert-butyl (3-amino-4-methylphenyl)carbamate (Method 230, 3.08 g, 0.0135 mmol), 6-bromo-3-methylquinazolin-4(3H)-one (Method 104; 3.24 g, 0.0135 mmol), Cs₂CO₃ (13.20 g, 0.0405 mol, 3.0 equiv), BINAP (841 mg, 1.35 mmol, 5 mol %) in dioxane (50 ml) was treated with Pd₂(dba)₃ (618 mg, 0.675 mmol). The reaction mixture was heated to 80° C. for 12 h. The reaction was then quenched with 10% NaOH(aq) and extracted with EtOAc. The organics were dried with NaCl(sat) and then Na₂SO₄(s). The organics were removed under reduced pressure and the resulting solid was treated with DCM (100 ml). The resulting precipitate was collected by vacuum filtration (3.00 g, 58%); m/z 387.

### Method 232

### 6-[(5-Amino-2-methylphenyl)amino]-3-methylquinazolin-4(3H)-one

A stirred mixture of tert-butyl {4-methyl-3-[(3-methyl-4-oxo-3,4-dihydroquinazolin-6-yl)amino]phenyl}carbamate (Method 231; 3.00 g, 7.78 mmol) in DCM (30 ml) was treated with TFA (30 ml). The solvents were removed under reduced pressure. The resulting solid was treated with 10% NaOH(aq) and extracted with EtOAc. The organics were dried with NaCl(sat) and then Na₂SO₄(s). The organics were then removed under reduced pressure (2.18 g, 99%); m/z 280.

### Method 233

### Methyl 3-[2-(dimethylamino)-1,1-dimethyl-2-oxoethyl]benzoate

2-(3-Bromophenyl)-N,N,2-trimethylpropanamide (Method 113; 202 mg, 0.748 mmol), MeOH (35 μl, 7.48 mmol, 10.00 equiv), Pd(OAc)₂ (17 mg, 0.075 mmol, 10 mol %), Mo(CO)₆ (296 mg, 1.12 mmol, 1.5 equiv), Cs₂CO₃ (365 mg, 1.12 mmol, 1.5 equiv) and BINAP (47 mg, 0.075 mmol, 10 mol %) in toluene-CH₃CN 1:1 (2 ml) was heated at 90° C. under Ar for 12 h. The reaction was quenched with 10% NaOH and extracted with EtOAc. The organics were dried with NaCl(sat) and Na₂SO₄(s) and then removed under reduced pressure. The residue was then purified by column chromatography utilizing an ISCO system (EtOAc-hexane) to give 50 mg (27%) of the desired product; m/z 250.

### Method 234

### Methyl 4-[(cyclopropylamino)methyl]-3-(trifluoromethyl)benzoate

A suspension of methyl 4-(bromomethyl)-3-(trifluoromethyl)benzoate (Method 158; 0.85 g, 2.86 mmol), cyclopropylamine (0.82 g, 41.3 mmol) and K₂CO₃ (1.19 g, 8.58 mmol) in CH₃CN (15 ml) was stirred at 45° C. for 15 h. The reaction mixture was concentrated under reduced pressure and purified on silica gel; m/z 274.

### Method 235

### Methyl 3-cyclopropylbenzoate

To a 100 ml round bottom flask charged with a magnetic stir bar and DCM (20 ml) was added 12.3 ml of diethyl zinc (1M in hexanes). The reaction mixture was cooled to 0° C. and trifluoroacetic acid (1.40 g, 12.3 mmol) was added dropwise via syringe. The reaction was stirred at this temperature for 20 mins followed by the addition of CH₂I₂ (3.30 g, 12.3 mmol). The reaction mixture was stirred for 20 mins before methyl 3-vinylbenzoate (1.00 g, 6.16 mmol) was added. The reaction was then allowed to warm to 25° C. with stirring for 3 h. before being quenched by the addition of ˜50 ml of saturated aqueous NH₄Cl. The mixture was poured into a separatory funnel and the aqueous phase was further extracted with DCM (3×50 ml). The combined organic extract was dried with MgSO₄ and concentrated in vacuo to yield the crude reaction product which was purified on 120 g SiO₂ using hexanes-EtOAc 10:1 as eluent giving 1.01 g of the title compound as a colourless oil (94%); m/z 177.

1. A compound of formula (I): 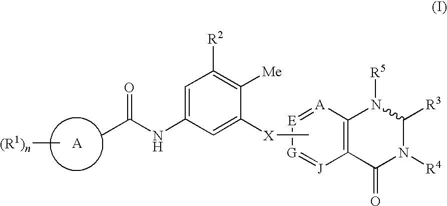
wherein:
Ring A is carbocyclyl or heterocyclyl; wherein if said heterocyclyl contains an —NH— moiety that nitrogen may be optionally substituted by a group selected from R⁶;
R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, N—(C₁₋₆alkoxy)sulphamoyl, N—(C₁₋₆alkyl)-N—(C₁₋₆alkoxy)sulphamoyl, C₁₋₆alkylsulphonylamino, carbocyclyl-R⁷— or heterocyclyl-R⁸—; wherein R¹ may be optionally substituted on carbon by one or more R⁹; and wherein if said heterocyclyl contains an —NH— moiety that nitrogen may be optionally substituted by a group selected from R¹⁰;
n is selected from 0-4; wherein the values of R¹ may be the same or different;
R² is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹¹— or heterocyclyl-R¹²—; wherein R² may be optionally substituted on carbon by one or more R¹³; and wherein if said heterocyclyl contains an —NH— moiety that nitrogen may be optionally substituted by a group selected from R¹⁴;
X is NR¹⁵ or O;
one of A, E, G and J is C which is attached to X of formula (I); the other three are independently selected from CR¹⁶ or N;
R³ and R¹⁶ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R³ and R¹⁶ independently of each other may be optionally substituted on carbon by one or more R¹⁹; and wherein if said heterocyclyl contains an —NH— moiety that nitrogen may be optionally substituted by a group selected from R²⁰;
R⁴, R⁵ and R¹⁵ are independently selected from hydrogen, C₁₋₆alkyl, C₁₋₆alkanoyl, C₁₋₆alkylsulphonyl, C₁₋₆alkoxycarbonyl, carbamoyl, carbocyclyl, heterocyclyl, N—(C₁₋₆alkyl)carbamoyl and N,N—(C₁₋₆alkyl)carbamoyl; wherein R⁴, R⁵ and R¹⁵ independently of each other may be optionally substituted on carbon by one or more R²¹;
the bond [symbol] between the —NR⁵— and —CR³— of formula (I) is either (i) a single bond wherein R⁵ is as defined above, or (ii) a double bond wherein R⁵ is absent;
R⁹, R¹³, R¹⁹ and R²¹ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R²²— or heterocyclyl-R²³—; wherein R⁹, R¹³, R¹⁹ and R²¹ independently of each other may be optionally substituted on carbon by one or more R²⁴; and wherein if said heterocyclyl contains an —NH— moiety that nitrogen may be optionally substituted by a group selected from R²⁵;
R⁷, R⁸, R¹¹, R¹², R¹⁷, R¹⁸, R²² and R²³ are independently selected from a direct bond, —O—, —N(R²⁶)—, —C(O)—, —N(R²⁷)C(O)—, —C(O)N(R²⁸)—, —S(O)ₛ—, —SO₂N(R²⁹)— or —N(R³⁰)SO₂—; wherein R²⁶, R²⁷, R²⁸, R²⁹ and R³⁰ is hydrogen, C₁₋₆alkoxycarbonyl or C₁₋₆alkyl and s is 0-2;
R⁶, R¹⁰, R¹⁴, R²⁰ and R²⁵ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R²⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof.

2. A compound of formula (I), or a pharmaceutically acceptable salt thereof, as claimed in claim 1 wherein Ring A is phenyl, thienyl, pyridyl, thiazolyl, isoxazolyl, furyl, 1,3-benzodioxolyl, pyrazolyl, indolyl, 2,3-dihydrobenzofuranyl, imidazo[1,2-a]pyridinyl or pyrimidinyl; wherein said pyrazolyl may be optionally substituted on nitrogen by a group selected from R⁶; wherein R⁶ is C₁₋₆alkyl.

3. A compound of formula (I), or a pharmaceutically acceptable salt thereof, as claimed in claim 1 wherein:
R¹ is a substituent on carbon and is selected from halo, hydroxy, cyano, sulphamoyl, C₁₋₆alkyl, C₂₋₆alkenyl, C₂₋₆alkynyl, C₁₋₆alkoxy, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, N—(C₁₋₆alkyl)-N—(C₁₋₆alkoxy)sulphamoyl, carbocyclyl-R⁷— or heterocyclyl-R⁸—; wherein R¹ may be optionally substituted on carbon by one or more R⁹; and wherein if said heterocyclyl contains an —NH— moiety that nitrogen may be optionally substituted by a group selected from R¹⁰;
R⁹ is selected from halo, cyano, hydroxy, carboxy, C₁₋₆alkyl, C₁₋₆alkoxy, N,N—(C₁₋₆alkyl)₂amino, N—(C₁₋₆alkyl)carbamoyl, N,N—(C₁₋₆alkyl)₂carbamoyl, C₁₋₆alkylS(O)ₐ wherein a is 0 to 2, carbocyclyl-R²²— or heterocyclyl-R²³—; wherein R⁹ may be optionally substituted on carbon by one or more R²⁴; and wherein if said heterocyclyl contains an —NH— moiety that nitrogen may be optionally substituted by a group selected from R²⁵;
R⁷, R⁸, R²² and R²³ are independently selected from a direct bond, —O—, —N(R²⁶)—, —C(O)—, —S(O)ₛ— or —N(R³⁰)SO₂—; wherein R²⁶ and R³⁰ are independently selected from hydrogen or C₁₋₆alkoxycarbonyl; and s is 2;
R¹⁰ and R²⁵ are independently selected from C₁₋₆alkyl;
R²⁴ is hydroxy.

4. A compound of formula (I), or a pharmaceutically acceptable salt thereof, as claimed in claim 1 wherein n is selected from 0-2; wherein the values of R¹ may be the same or different.

5. A compound of formula (I), or a pharmaceutically acceptable salt thereof, as claimed in claim 1 wherein R² is hydrogen.

6. A compound of formula (I), or a pharmaceutically acceptable salt thereof, as claimed in claim 1 wherein:
X is NR¹⁵ or O; wherein
R¹⁵ is selected from hydrogen or C₁₋₆alkyl; wherein R¹⁵ may be optionally substituted on carbon by one or more R²¹;
R²¹ is selected from carbocyclyl-R²²—;
R²² is a direct bond.

7. A compound of formula (I), or a pharmaceutically acceptable salt thereof, as claimed in claim 1 wherein one of A, E, G and J is C which is attached to X of formula (I); the other three are all CR¹⁶ or two are CR¹⁶ and one is N; wherein R¹⁶ is hydrogen.

8. A compound of formula (I), or a pharmaceutically acceptable salt thereof, as claimed in claim 1 wherein:
R³ is selected from hydrogen, C₁₋₆alkyl, N—(C₁₋₆alkyl)amino, N,N—(C₁₋₆alkyl)₂amino or C₁₋₆alkylS(O)ₐ wherein a is 0; wherein R³ may be optionally substituted on carbon by one or more R¹⁹; wherein
R¹⁹ is hydroxy.

9. A compound of formula (I), or a pharmaceutically acceptable salt thereof, as claimed in claim 1 wherein:
R⁴ is selected from hydrogen, C₁₋₆alkyl or carbocyclyl; wherein R⁴ may be optionally substituted on carbon by one or more R²¹;
R²¹ is selected from hydroxy, amino, C₁₋₆alkoxycarbonylamino, carbocyclyl-R²²— or heterocyclyl-R²³—; wherein R²¹ may be optionally substituted on carbon by one or more R²⁴; and wherein if said heterocyclyl contains an —NH— moiety that nitrogen may be optionally substituted by a group selected from R²⁵;
R²² and R²³ are a direct bond;
R²⁴ is methyl; and
R²⁵ is C₁₋₆alkyl or benzyloxycarbonyl.

10. A compound of formula (I), or a pharmaceutically acceptable salt thereof, as claimed in claim 1 wherein the bond [symbol] between the —NR⁵— and —CR³— of formula (I) is a double bond wherein R⁵ is absent.

11. A compound of formula (I): 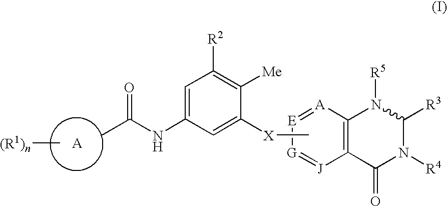
wherein:
Ring A is phenyl, thien-2-yl, thien-3-yl, pyrid-2-yl, pyrid-3-yl, pyrid-4-yl, thiazol-4-yl, isoxazol-3-yl, 1,3-benzodioxol-5-yl, fur-2-yl, 1-methylpyrazol-3-yl, 1-methylpyrazol-5-yl, 1-t-butylpyrazol-5-yl, indol-5-yl, indol-6-yl, 2,3-dihydrobenzofuran-7-yl, imidazo[1,2-a]pyridin-2-yl or pyrimidin-4-yl;
R¹ is a substituent on carbon and is selected from fluoro, chloro, bromo, hydroxy, cyano, sulphamoyl, methyl, trifluoromethyl, cyclopropylaminomethyl, methylthiomethyl, mesylmethyl, dimethylaminomethyl, 1-(cyclopropyl)-1-hydroxymethyl, N-cyclopropyl-N-(t-butoxycarbonyl)aminomethyl, 1-methylpiperazin-4-ylmethyl, 1-hydroxy-1-cyclopropylethyl, 1-methyl-1-cyanoethyl, 2-methoxy-1,1-dimethylethyl, 1-carboxy-1-methylethyl, 1,1-difluoroethyl, 2-(dimethylamino)-1,1-dimethyl-2-oxoethyl, 3-(dimethylamino)propyl, 1,1-dimethylpropyl, t-butyl, methoxy, N-methylcarbamoylmethoxy, 2-(dimethylamino)ethoxy, 2-(pyrrolidin-1-yl)ethoxy, 2-(methoxy)ethoxy, 2-(1-methylpyrrolidin-2-yl)ethoxy, 2-(piperidin-1-yl)ethoxy, 2-(azepan-1-yl)ethoxy, 2-(morpholino)ethoxy, 3-(1-methylpiperazin-4-yl)propoxy, methoxycarbonyl, morpholinocarbonyl, N,N-dimethylsulphamoyl, N-(2,3-dihydroxypropyl)-N-methylsulphamoyl, N-(methyl)-N-(methoxy)sulphamoyl, 1-methylpiperidin-4-yloxy, N,N-dimethylcarbamoyl, cyclopropyl, piperidin-1-yl, morpholino, 1-cyclopropylethenyl, 3-(4-methylpiperazin-1-yl)prop-1-yn-1-yl, 3,3-dimethylbut-1-yn-1-yl, cyclopropylethynyl, 3-hydroxy-3-methylbut-1-yn-1-yl, 1,1-dimethylprop-2-yn-1-yl, 3-(dimethylamino)prop-1-yn-1-yl, mesyl, cyclopropylaminosulphonyl, azetidin-1-ylsulphonyl, morpholinosulphonyl, tetrahydrofur-2-ylmethylaminosulphonyl, 2-(hydroxymethyl)piperidin-1-ylsulphonyl, 3-(hydroxymethyl)piperidin-1-ylsulphonyl or 4-(hydroxymethyl)piperidin-1-ylsulphonyl;
n is selected from 0-2; wherein the values of R¹ may be the same or different;
R² is hydrogen;
X is NR¹⁵ or O;
one of A, E, G and J is C which is attached to X of formula (I); the other three are all CR¹⁶ or two are CR¹⁶ and one is N;
R³ is selected from hydrogen, methyl, N-(2-hydroxyethyl)amino, N,N-dimethylamino or methylthio;
R⁴ is selected from hydrogen, methyl, 1-methylpiperidin-3-ylmethyl, cyclopropylmethyl, 2,2-dimethyl-1,3-dioxolan-4-ylmethyl, piperidin-4-ylmethyl, 1-benzyloxycarbonylpipidin-4-ylmethyl, ethyl, 2-hydroxyethyl, 3-aminopropyl, 3-(t-butoxycarbonylamino)propyl, 3-morpholinopropyl, 2,3-dihydroxypropyl and cyclopropyl;
the bond [symbol] between the —NR⁵— and —CR³— of formula (I) is a double bond wherein R⁵ is absent; and
R¹⁵ is selected from hydrogen, methyl or cyclopropylmethyl;
R¹⁶ is hydrogen;
or a pharmaceutically acceptable salt thereof.

12. A compound of formula (I): 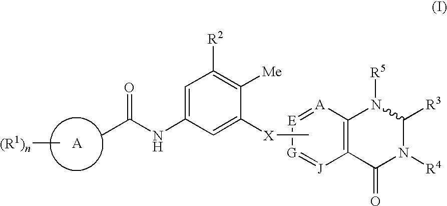
selected from:
3-(1,1-dimethylprop-2-yn-1-yl)-N-{4-methyl-3-[(3-methyl-4-oxo-3,4-dihydroquinazolin-6-yl)amino]phenyl}benzamide;
3-(1-cyano-1-methylethyl)-N-{4-methyl-3-[(3-methyl-4-oxo-3,4-dihydroquinazolin-6-yl)amino]phenyl}benzamide;
3-(1-cyano-1-methylethyl)-5-fluoro-N-{4-methyl-3-[(3-methyl-4-oxo-3,4-dihydroquinazolin-6-yl)amino]phenyl}benzamide;
3-(1-cyano-1-methylethyl)-5-[(dimethylamino)methyl]-N-{4-methyl-3-[(3-methyl-4-oxo-3,4-dihydroquinazolin-6-yl)amino]phenyl}benzamide;
4-dimethylaminomethyl-N-[4-methyl-3-(3-methyl-4-oxo-3,4-dihydro-quinazolin-6-ylamino)-phenyl]-3-trifluoromethyl-benzamide;
2-(1-cyano-1-methylethyl)-N-{4-methyl-3-[(3-methyl-4-oxo-3,4-dihydroquinazolin-6-yl)amino]phenyl}isonicotinamide;
3-(1-cyano-1-methylethyl)-2-fluoro-N-{4-methyl-3-[(3-methyl-4-oxo-3,4-dihydroquinazolin-6-yl)amino]phenyl}benzamide;
N-(3-{[3-(3-aminopropyl)-4-oxo-3,4-dihydroquinazolin-6-yl]amino}-4-methyl phenyl)-3-(1-cyano-1-methylethyl)benzamide;
3-{[methoxy(methyl)amino]sulfonyl}-N-{4-methyl-3-[(3-methyl-4-oxo-3,4-dihydroquinazolin-6-yl)amino]phenyl}benzamide; and
3-tert-butyl-N-{4-methyl-3-[(3-methyl-4-oxo-3,4-dihydroquinazolin-6-yl)amino]phenyl}benzamide;
or a pharmaceutically acceptable salt thereof.

13. A process for preparing a compound of formula (I) or a pharmaceutically acceptable salt thereof, as claimed in claim 1 which process, wherein variable are, unless otherwise specified, as defined in claim 1, comprises of:
Process a) reacting an amine of the formula (II) 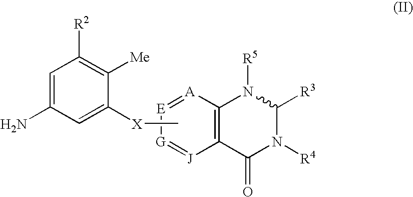
with an acid of formula (III): 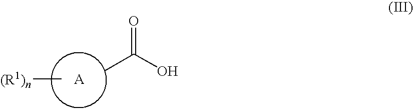
or an activated acid derivative thereof; or
Process b) reacting a compound of formula (IV): 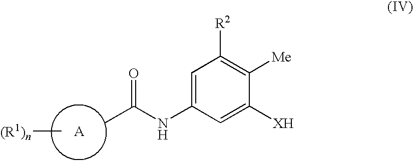
with an compound of formula (V): 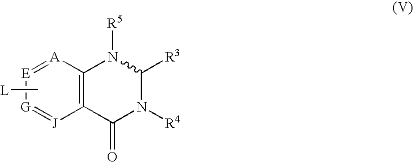
wherein L is a displaceable group; or
Process c) reacting a compound of formula (VI) wherein L is a displaceable group: 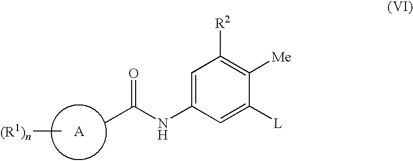
wherein L is a displaceable group; with an compound of formula (VII): 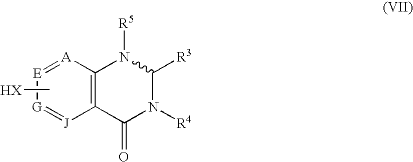
or
Process d) for compounds of formula (I) wherein R⁴ is not hydrogen; reacting a compound of formula (I) wherein R⁴ is hydrogen with a compound of formula (VIII):
R⁴-L (VIII)
wherein L is a displaceable group and R⁴ is not hydrogen; or
Process e) for compounds of formula (I) wherein X is NR¹⁵ and R¹⁵ is —CH₂—C₂₋₆alkyl optionally substituted on carbon by one or more R²¹; reacting a compound of formula (I) wherein X is NR¹⁵ and R¹⁵ is hydrogen with a compound of formula (IX): 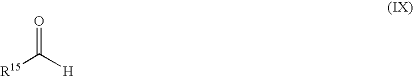
wherein R¹⁵ is C₁₋₅alkyl optionally substituted on carbon by one or more R²¹; or
Process f) for compounds of formula (I) wherein X is NR¹⁵ and R¹⁵ is not hydrogen; reacting a compound of formula (I) wherein X is NR¹⁵ and R¹⁵ is hydrogen with a compound of formula (X):
R¹⁵-L (X)
wherein L is a displaceable group and R¹⁵ is not hydrogen;
and thereafter if necessary:
i) converting a compound of the formula (I) into another compound of the formula (I);
ii) removing any protecting groups;
iii) forming a pharmaceutically acceptable salt.

14. A pharmaceutical composition which comprises a compound of the formula (I), or a pharmaceutically acceptable salt thereof, as claimed in claim 1, in association with a pharmaceutically-acceptable diluent or carrier.

15-18. (canceled)

19. A method for producing a B-Raf inhibitory effect in a warm-blooded animal, such as man, in need of such treatment which comprises administering to said animal an effective amount of a compound of formula (I), or a pharmaceutically acceptable salt thereof, as claimed in claim 1.

20. A method for producing an anti-cancer effect in a warm-blooded animal, such as man, in need of such treatment which comprises administering to said animal an effective amount of a compound of formula (I), or a pharmaceutically acceptable salt thereof, as claimed in claim 1.

21. A method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof, as claimed in claim 1.

22-24. (canceled)
